(19) United States
(12) Patent Application Publication
(10) Pub. No.: US 2026/0337245 A1
(43) Pub. Date: Oct. 29, 2026

(54) POLYPEPTIDE OR ANALOG THEREOF AND USE THEREOF

(71) Applicant: Shenzhen Bay Laboratory, Shenzhen (CN)

(72) Inventors: Nan ZHENG, Shenzhen (CN); Leiming WANG, Shenzhen (CN); You WANG, Shenzhen (CN); Weijun SHEN, Shenzhen (CN)

(21) Appl. No.: 19/565,582

(22) Filed: Mar. 13, 2026

Related U.S. Application Data
(63) Continuation of application No. PCT/CN2025/073377, filed on Jan. 20, 2025.

(30) Foreign Application Priority Data
Mar. 19, 2024 (CN) 202410315412.8

(51) Int. Cl. C07K 14/585; A61K 9/00; A61K 38/00; A61P 3/00

(52) U.S. Cl. CPC C07K 14/585; A61K 9/0019; A61K 38/00; A61P 3/00

(57) ABSTRACT

The present disclosure discloses a polypeptide, derivative thereof or pharmaceutically acceptable salt thereof. The polypeptide, derivative thereof, or pharmaceutically acceptable salt thereof has a structure (U—X₁SHX₄SX₆X₇X₈X₉X₁₀X₁₁X₁₂X₁₃X₁₄X₁₅X₁₆X₁₇X₁₈X₁₉X₂₀DX₂₂PX₂₄TDVGAGSX₃₂—NH₂) as represented by formula (I) and a modifying group. The structure represented by formula (I) includes two amino acids Z₁ or two amino acids Z₂. The sites of the two amino acids Z₁ or the two amino acids Z₂ are Xᵢ and Xᵢ₊₇ respectively, where i is any integer between 6 and 13. The modifying group is bound to the two amino acids Z₁ or the two amino acids Z₂ via covalent bonds. The polypeptide or derivative thereof has certain activation activity on amylin receptor and calcitonin receptor simultaneously, and has long in-vivo half-life through modification of side chain modifying groups so as to support low-dose and low-frequency administration, and can be used for preventing or treating metabolic related diseases.

### CROSS-REFERENCE TO RELATED APPLICATIONS

This application is a continuation of International Application No. PCT/CN2025/073377, filed on Jan. 20, 2025, which claims priority to Chinese Patent Application No. 202410315412.8, filed on Mar. 19, 2024. The disclosures of the aforementioned applications are hereby incorporated by reference in their entireties.

### STATEMENT REGARDING SEQUENCE LISTING

A Sequence Listing associated with this application is being filed concurrently herewith in XML format and is hereby incorporated by reference into the present specification. The XML file containing the Sequence listing is titled “Sequence_Listing.xml”, was created on Jun. 17, 2026, and is 102,320 bytes in size.

### FIELD

The present disclosure belongs to the field of bio-pharmaceuticals, and in particular, relates to a polypeptide or analog thereof and use thereof, and more particularly, to a polypeptide, derivative thereof, pharmaceutically acceptable salt, or pharmaceutical composition that simultaneously exert activating activity on both an amylin receptor (AMYR) and a calcitonin receptor (CTR), and use thereof.

### BACKGROUND

The continued increase in the prevalence rates of obesity and type 2 diabetes mellitus (T2DM) has led to lack of effective measures. Meanwhile, patients with obesity and T2D are often at a high risk of developing cardiovascular diseases and affected by other metabolic comorbidities, complicating the safe use of drugs. In addition to changes in dietary and lifestyle, bariatric surgery can effectively relieve obesity and T2D at present, but the surgery involves risks and irreversible physical alterations. Therefore, there is a need to develop novel and more effective pharmacological therapeutic approaches to achieve blood glucose control and weight management.

Amylin is a pancreatic hormone composed of 37 amino acids, which is co-secreted with insulin by pancreatic R cells and plays a role in satiety, gastric emptying and glucagon secretion. Amylin controls satiety signals by directly acting on amylin receptor (AMYR) in the brain. Three subtypes of amylin receptors, namely Amy1R, Amy2R and Amy3R have been identified to date. Studies have shown that Amy1R is related to lipid metabolism, while Amy3R is responsible for blood glucose regulation. Amylin is also a potent inhibitor of gastric emptying, which can further inhibit glucagon secretion through central mechanisms.

Calcitonin is a 32-amino-acid peptide secreted by the C cells thyroid gland. Calcitonin acts on calcitonin receptors (CTR) and reduces blood calcium by inhibiting osteoclasts and promoting renal calcium excretion.

Studies have shown that synergistic activation of amylin receptors and calcitonin receptors has a positive metabolic promoting effect and can effectively reduce blood glucose levels and body weight. The development of long-acting dual agonists of amylin receptor and calcitonin receptor is expected to provide an effective drug for the treatment of metabolic diseases, including obesity, type 2 diabetes mellitus, non-alcoholic steatohepatitis (NASH), dyslipidemia, etc. Currently, no long-acting dual agonists of amylin receptors and calcitonin receptors (DACRA) have been approved. Therefore, there is an urgent need to develop a polypeptide or analog thereof that can exert activating activity on both amylin receptor (AMYR) and calcitonin receptor (CTR).

### SUMMARY

The present disclosure aims to solve at least one of the technical problems in the prior art at least to a certain extent. Therefore, the present disclosure provides a polypeptide or analog thereof that exert activating activity on both the amylin receptor (AMYR) and the calcitonin receptor (CTR).

The present disclosure is accomplished on the basis of the following findings of the inventors.

Some long-acting amylin analogs are in a clinical research stage and have shown certain advantages in the treatment of metabolic diseases. Pramlintide, an amylin analog, has been approved for use in combination with insulin therapy to improve insulin sensitivity and assist in lowering blood glucose in diabetic patients. However, in-vivo half-life of this drug is less than 1 hour, so patients need to inject it multiple times a day with meals, resulting in inconvenience. In addition, amylin analogs are prone to cause fibrillation aggregation both in vivo and in vitro due to their structural characteristics, resulting in stringent storage conditions and potential immunogenic risks.

Salmon calcitonin (sCT) is used for diseases such as hypercalciemia, osteoporosis, and Paget's disease of bone. Recent studies have shown that calcitonin also has other metabolic benefits, such as affecting insulin sensitivity, reducing a gastric emptying rate, and promoting satiety, etc. However, its short in-vivo half-life necessitates multiple daily subcutaneous injections, resulting in poor patient compliance. In addition, sCT has poor stability, and is prone to aggregation and precipitation, which can trigger immunogenic responses and raise safety concerns.

The development of polypeptide drugs is often hindered by factors such as short half-life and low bioavailability. As a result, patients have to receive higher doses of therapeutic drugs more frequently, which may lead to reduced compliance, increased cost, and elevated risks of side effects. Therefore, it is necessary to develop therapeutic agents with an extended half-life.

Based on this, the method of the present disclosure locks a dominant binding conformation of a target polypeptide by introducing a residue (including but not limited to cysteine (C or c) or lysine (K or k)) with a side chain containing —SH or —NH₂ at two mutation sites in a polypeptide chain, and performing dual-site side-chain fixed modification through macrocyclization. Compared with traditional polypeptide drugs, this strategy can reduce the tendency of aggregation and fibrillation, which is conducive to further improving the stability and receptor binding activity of the target polypeptide.

Therefore, in an aspect of the present disclosure, the present disclosure provides a polypeptide, derivative thereof, pharmaceutically acceptable salt of the polypeptide. According to an embodiment of the present disclosure, the polypeptide, derivative thereof, or pharmaceutically acceptable salt thereof has a structure represented by formula (I):

U—X₁SHX₄SX₆X₇X₈X₉X₁₀X₁₁X₁₂X₁₃X₁₄X₁₅X₁₆X₁₇X₁₈X₁₉X₂₀DX₂₂PX₂₄TDVGAGSX₃₂—NH₂ (I), wherein:
U is independently hydrogen, acetyl, or benzoyl;
X₁ is A or K;
X₄ is L or αMeL;
X₆ is Z₁, Z₂ or T;
X₇ is Z₁, Z₂ or A;
X₈ is Z₁, Z₂ or V;
X₉ is Z₁, Z₂ or L;
X₁₀ is Z₁, Z₂ or G;
X₁₁ is Z₁, Z₂, homoR, Orn or R;
X₁₂ is Z₁, Z₂ or L;
X₁₃ is Z₁, Z₂ or S;
X₁₄ is Z₁, Z₂ or A;
X₁₅ is Z₁, Z₂ or E;
X₁₆ is Z₁, Z₂ or L;
X₁₇ is Z₁, Z₂ or H;
X₁₈ is Z₁, Z₂, K, αMeK or Orn;
X₁₉ is Z₁, Z₂ or L;
X₂₀ is Z₁, Z₂ or Aib;
X₂₂ is Y or αMeF;
X₂₄ is R, N-Me-R, homoR, norR, Q or r;
X₃₂ is P, Hyp, cis-P(4-NH₂), trans-P(4-NH₂) or p;
wherein the structure represented by formula (I) includes two amino acids Z₁ or two amino acids Z₂, the sites of the two amino acids Z₁ or the two amino acids Z₂ are X₁ and Xᵢ₊₇ respectively, and i is any integer between 6 and 13;
wherein each amino acid Z₁ is independently selected from C, c, αMeC, HoC, Hoc, Pen or N-Me-C; and
wherein each amino acid Z₂ is independently selected from K, k, Dap, Dab, Orn, HomoK, N-Me-K, N-Me-k, αMeK or αMek.

The polypeptide, the derivative thereof, or the pharmaceutically acceptable salt thereof (referred to as a polypeptide or an analog thereof) according to an embodiment of the present disclosure is a dual agonist polypeptide analog, which can simultaneously exhibit a certain balanced or unbalanced activating activity on amylin receptor (AMYR) and calcitonin receptor (CTR). The polypeptide, derivative thereof, or pharmaceutically acceptable salt thereof may be used to prevent or treat metabolic disorder syndromes, such as T2DM, obesity, hyperlipidemia, NAFLD and NASH; and also has other potential applications in the treatment of diseases, such as Alzheimer's disease (AD), alcohol or drug addiction-related symptoms or diseases, etc.

In a second aspect of the present disclosure, the present disclosure provides a pharmaceutical composition. According to an embodiment of the present disclosure, the pharmaceutical composition includes the polypeptide, derivative thereof or pharmaceutically acceptable salt thereof in the first aspect. As previously known, the polypeptide, derivative thereof or pharmaceutically acceptable salt thereof (referred to as a polypeptide or an analog thereof) is a dual agonist polypeptide analog, which can simultaneously exhibit a certain balanced or unbalanced activating activity on both amylin receptor (AMYR) and calcitonin receptor (CTR). Therefore, a medicament including the polypeptide, derivative thereof or pharmaceutically acceptable salt thereof is effective in preventing or treating amylin receptor- and/or calcitonin receptor-related diseases.

In a third aspect of the present disclosure, the present disclosure proposes a combination of medicament or kit. According to an embodiment of the present disclosure, the combination of medicament or kit includes the polypeptide, derivative thereof or pharmaceutically acceptable salt thereof according to the first aspect, or the pharmaceutical composition according to the second aspect as a first active ingredient; and a second active ingredient, wherein the second active ingredient includes a medicament for preventing and/or treating diseases. The combination of medicament or kit according to an embodiment of the present disclosure can further improve a therapeutic effect on amylin receptor- and/or calcitonin receptor-related diseases.

In a fourth aspect of the present disclosure, the present disclosure provides a use of the polypeptide, derivative thereof or pharmaceutically acceptable salt thereof according to the first aspect, or the pharmaceutical composition according to the second aspect, or the combination of medicament or kit according to the third aspect in the treatment or prevention of amylin receptor- and/or calcitonin receptor-related diseases; and/or in the manufacture of a medicament for treating or preventing an amylin receptor- and/or calcitonin receptor-related disease.

In a fifth aspect of the present disclosure, the present disclosure proposes a method for treating or preventing an amylin receptor- and/or calcitonin receptor-related disease. According to an embodiment of the present disclosure, the method includes: administrating to a subject a pharmaceutically acceptable amount of the polypeptide, derivative thereof or pharmaceutically acceptable salt thereof according to the first aspect, the pharmaceutical composition according to the second aspect or the combination of medicament or kit according to the second aspect.

The additional aspects and advantages of the present disclosure will be partially given in the following description, and some will become obvious from the following description, or be learned through the practice of the present disclosure.

### BRIEF DESCRIPTION OF DRAWINGS

The above and/or additional aspects and advantages of the present disclosure will become obvious and easy to understand from the description of the examples in conjunction with the following accompanying drawings, in which:

FIG. 1 is a schematic diagram of a Staple linker synthesis process in Example 1 of the present disclosure;

FIG. 2 is a graph showing the binding activity results of different polypeptide derivatives to AMY3R in Test Example 1 of the present disclosure;

FIG. 3 is a graph showing the binding activity results of different polypeptide derivatives to CTR in Test Example 1 of the present disclosure;

FIG. 4A to 4C are PK curve graphs of different polypeptide derivatives in rats in Test Example 2 of the present disclosure;

FIG. 5 is a PK curve graph of different polypeptide derivatives in Cynomolgus macaques in Test Example 2 of the present disclosure;

FIG. 6 shows the results of changes in body weight and food intake after a single administration of polypeptide derivatives in an SD rat model in Test Example 3 of the present disclosure;

FIG. 7 shows the results of changes in body weight and food intake after a single administration of polypeptide derivatives in a Diet-Induced Obesity (DIO) mouse model in Test Example 4 of the present disclosure, where the positive control drug is Cagrilintide;

FIG. 8 shows the results of changes in body weight and food intake after a single administration of polypeptide derivatives combined with Semaglutide in a DIO mouse model in Test Example 5 of the present disclosure, where the positive control drug is Cagrilintide or Semaglutide at the same dose;

FIG. 9 shows the results of changes in body weight, blood glucose and food intake after continuous administration of polypeptide derivatives in a Zucker Diabetic Fatty (ZDF) rat model in Test Example 6 of the present disclosure; and

FIG. 10 shows the results of the aggregation and precipitation stability test of polypeptide derivatives in Test Example 9 of the present disclosure, where the positive control is salmon calcitonin.

### DESCRIPTION OF EMBODIMENTS

The examples of the present disclosure are described in detail below. The examples described below are exemplary, and are only intended to explain the present disclosure, rather than being construed as limitations to the present disclosure.

It should be noted that the terms “first” and “second” are only for the purpose of description and should not be construed as indicating or implying relative importance or implicitly indicating the number of technical features indicated. Thus, the features defined with “first” and “second” may include one or more of the features either explicitly or implicitly. Further, in the description of the present disclosure, unless otherwise stated, the term “a plurality of” means two or more.

### DETAILED DESCRIPTION OF THE PRESENT DISCLOSURE

### Definitions and General Terms

The term “comprising” or “including” herein is an open-ended expression, that is, including the content indicated in the present disclosure, but not excluding other aspects.

The term “pharmaceutically acceptable” herein means that a substance or composition must be chemically and/or toxicologically compatible with other components containing a polypeptide or a derivative thereof and/or with mammals to be treated. Preferably, the “pharmaceutically acceptable” of the present disclosure means a substance or composition used in animals, particularly in human as approved by federal regulatory agencies or national governments, or listed in the United States Pharmacopoeia or other generally recognized pharmacopoeias.

The term “pharmaceutically acceptable salt” herein refers to an organic and inorganic salt of a polypeptide or a derivative thereof of the present disclosure. Pharmaceutically acceptable salts are well known in the fields, as described in the literature: S. M. Berge et al., describe pharmaceutically acceptable salts in detail in J. Pharmaceutical Sciences, 1977, 66: 1-19.

Amino acids herein are represented by regular single- and three-letter codes for natural amino acids, as well as generally accepted three-letter codes for other α-amino acids. For example, α-aminoisobutyric acid may be represented by the code Aib or B. Unless otherwise specified, amino acid residues expressed by all uppercase letters in the present disclosure are in an L-configuration, and amino acid residues expressed by lowercase letters are in an D-configuration.

The term “Aib” herein has a structural formula of

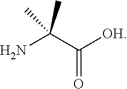

The term “αMeF” herein has a structural formula of

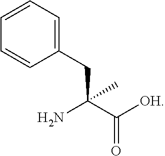

The term “Orn” herein has a structural formula of

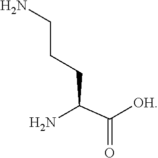

The term “αMeL” herein has a structural formula of

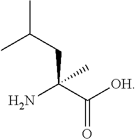

The term “MeK” herein has a structural formula of

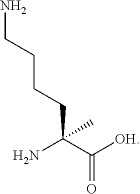

The term “N-Me-R” herein has a structural formula of

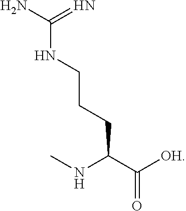

The term “Hyp” herein has a structural formula of

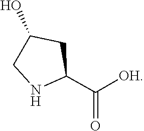

The term “homoR” herein has a structural formula of

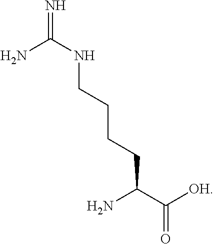 [SMILES: N=C(N)NCCCC[C@H](N)C(=O)O]

The term “cis-P(4-NH₂)” herein has a structural formula of

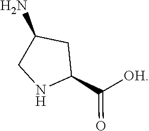

The term “trans-P(4-NH₁₂)” herein has a structural formula of

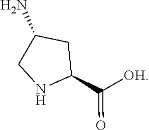

The term “αMeC” herein has a structural formula of

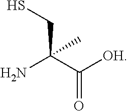

The term “HoC” herein has a structural formula of

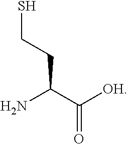

The term “Hoc” herein has a structural formula of

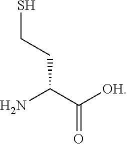

The term “Pen” herein has a structural formula of

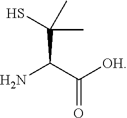

The term “N-Me-C herein has a structural formula of

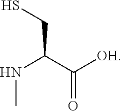

The term “Dab” herein has a structural formula of

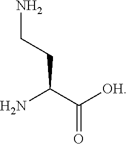

The term “Dap” herein has a structural formula of

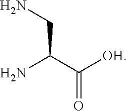

The term “homoK” herein has a structural formula of

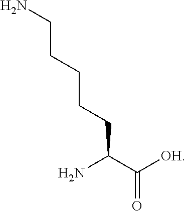 [SMILES: NCCCCC[C@H](N)C(=O)O]

The term “αMek” herein has a structural formula of

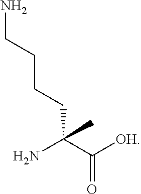

The term “N-Me-K” has a structural formula of

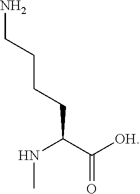

The term “optionally”, “optional” or “option” herein generally means that the subsequent event or condition may but need not occur necessarily and this description includes a circumstance where the event or condition occurs, and a circumstance where the event or condition does not occur.

The terms “optionally substituted”, “optionally substituted with” and “substituted or unsubstituted” herein are used interchangeably. In general, the term “optionally”, whether it precedes the term “substituted” or not, means that one or more hydrogen atoms in a given structure are substituted with specific substituents. Unless otherwise indicated, an optional substituent group may be substituted at any substitutable position of the group. When more than one position in a given structural formula can be substituted with one or more substituents selected from a specific group, the substituents may be same or different at each position. The substituents may be, but are not limited to, F, Cl, Br, CN, OH, NH₂, NO₂, etc.

The term “one or more” herein (e.g., in the definition of substituents for a compound (modifying groups) of a general formula in the present disclosure) means “one, two, three, four or five, in particular one, two, three or four, more particularly one, two or three, and more particularly one or two”.

In addition, it should be noted that, unless otherwise expressly stated, the description “each . . . independently is”, “ . . . is each independently“and” . . . independently is” adopted in the present disclosure are interchangeable and should be understood in a broad sense. They may refer to situations where, in different groups, the specific options expressed by the same symbol do not affect one another, or to situations where, in the same group, the specific options expressed by the same symbol do not affect one another.

The term “halogen” herein refers to fluorine, chlorine, bromine or iodine atoms.

Herein, the minimum and maximum number of carbon atoms in a hydrocarbon group are indicated by prefixes. For example, the prefix Ca-b refers to containing “a” to “b” carbon atoms. Exemplarily, “C₁₋ₙ” means a linear or branched saturated/unsaturated carbon chain containing 1, 2, 3, 4, 5, . . . or n carbon atoms. It is further understood that “C₁₋ₙ” should be interpreted as any subrange included therein. For example, in C₁₋₂₀, C₁₋₂₀, C₁₋₁₈, C₁₀₋₈, C₁₂₋₁₈, C₁₄₋₁₈, C₁₋₁₀, C₁₋₆, C₁₋₃, C₁₋₂, C₂₋₁₀, C₂₋₉, C₂₋₈, C₂₋₇, C₂₋₆, C₂₋₅, C₂₋₄, C₂₋₃, C₃₋₁₀, C₃₋₉, C₃₋₈, C₃₋₇, C₃₋₆, C₃₋₅, C₃₋₄, C₄₋₁₀, C₄₋₉, C₄₋₈, C₄₋₇, C₄₋₆ and C₄₋₅ are included.

The term “alkyl” herein has a structural formula of

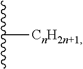

and alkyl may be either a linear alkyl or a branched alkyl. Exemplarily, the term “C₁₋₂₀ alkyl” refers to an alkyl having 1 to 20 carbon atoms; and the term “C₁₋₆ alkyl” refers to an alkyl having 1 to 6 carbon atoms.

The term “alkylene” herein has a structural formula of

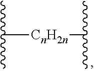

and alkylene may be either a linear alkylene or a branched alkylene. Exemplarily, the term “C₁₋₂₀ alkylene” refers to an alkylene having 1 to 20 carbon atoms; and the term “C₁₋₆ alkylene” refers to an alkylene having 1 to 6 carbon atoms.

The term “C₁₋₆ alkoxyl” herein refers to a C₁₋₆ alkyl containing a formula “—O-alkyl”, wherein the term “alkyl” is as defined above. For example, “C₁₋₆ alkoxyl” may be methoxyl, ethoxyl, n-propoxyl, isopropoxyl, n-butoxyl, isobutoxyl, tert-butoxyl, sec-butoxyl, pentyloxyl, isopentyloxyl, and n-hexoxyl, or isomers of the above groups. In particular, the “C₁₋₆ alkoxyl” may contain 1, 2, 3, 4 or 5 carbon atoms (“C₁₋₅ alkoxyl”), preferably 1, 2, 3 or 4 carbon atoms (“C₁₋₄ alkoxyl”).

The term “alkoxylene” refers to a group formed by removing one hydrogen atom from “alkoxyl”.

The term “aryl” herein refers to a carbocyclic system containing one, two, or three rings, wherein at least one ring system is aromatic, and each ring system contains a ring composed of 3 to 10 atoms. The aryl group is usually, but not necessary, bound to a parent molecule through an aromatic ring of the aryl group. The term “aryl” may be used interchangeably with the term “aromatic ring” or “aryl ring”. The aryl group may include phenyl, indenyl, naphthyl and anthryl. The aryl group may optionally be substituted with one or more substituents described in the present disclosure.

The term “arylene” herein refers to a group formed by removing one hydrogen atom from “aryl”.

The term “heteroaryl” herein refers to a carbocyclic system containing one, two, or three rings, wherein at least one ring system is aromatic. The heteroatoms refer to nitrogen atoms, oxygen atoms, sulfur atoms, etc. The “heteroaryl” usually refers to a monovalent saturated or partially unsaturated monocyclic or bicyclic ring system containing multiple ring atoms, including 1, 2 or 3 ring heteroatoms selected from N, O and S, with the remaining ring atoms being carbon.

The term “heteroarylene” herein refers to a group formed by removing one hydrogen atom from “heteroaryl”.

The

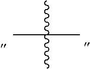

in the description of a group in the present disclosure is used to describe the substitution position of the group.

The term “—NH—CO—” or “—NH—C(═O)—” herein has a structural formula of

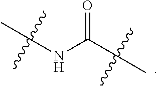 [SMILES: *NC(*)=O]

The “pharmaceutical composition” herein may refer to a formulation used for the treatment of a disease or for in vitro cell culture experiments. When used for the treatment of a disease, the term “pharmaceutical composition” generally refers to a unit dose form, which may be any type of formulation and may be prepared by means of any of the methods well-known in the pharmaceutical field. All methods include a step of combining an active ingredient with excipients constituting one or more accessory components. Typically, a composition is prepared by uniformly and sufficiently mixing the active polypeptide or a derivative thereof or a reactivator to a liquid excipient, a finely ground solid excipient, or both.

In a chemical structure of the ligand or compound described in the present disclosure, a bond “—” indicates an unspecified configuration. If there is chiral isomerism in the chemical structure, a bond “—” may be “ [symbol] ” or “ [symbol] ”, or contain both “ [symbol] ” and “ [symbol] ” simultaneously. Although for convenience all of these structural formulas are drawn in certain isomeric forms, the present disclosure may encompass all isomers, such as: tautomers, rotamers, geometric isomers, diastereoisomer, racemes, and enantiomers.

The term “pharmaceutically acceptable excipient” herein may include any solvent, including but not limited to pharmaceutically acceptable carriers, stabilizers, dispersants, co-solvents, plasticizers, solid excipients, diluents or other liquid excipients, etc., which are appropriate for specific target dosage forms.

The use of any conventional excipient is also within the scope contemplated by the present invention, except in cases where a conventional excipient is incompatible with the polypeptide of the present disclosure, derivative thereof or pharmaceutical composition thereof, or a medicament containing them, such as causing any adverse biological effects or interacting detrimentally with any other component of the pharmaceutically acceptable composition.

The pharmaceutical composition in the present disclosure includes formulations suitable for parenteral administration. The formulations may be conveniently present in unit dosage form and may be prepared by any method well-known in the pharmaceutical field. The amount of active ingredients that may be combined with excipients to prepare a single dose form is generally the amount of the polypeptide or derivative thereof that produces a therapeutic effect.

The term “agonist” herein refers to a substance (ligand) that activates the receptor type.

The term “treatment” herein refers to the achievement of a desired pharmacological and/or physiological effect. Such effect may be prophylactic in terms of complete or partial prevention of a disease or its symptoms, and/or may be therapeutic in terms of partial or complete cure of a disease and/or adverse effects caused by the disease. The term “treatment” as used herein encompasses diseases of mammals, particularly humans, including: (a) prevention the occurrence of a disease or condition in individuals susceptible to the disease but not yet diagnosed with it; (b) inhibition of a disease, e.g., arresting the development of the disease; or (c) alleviation of a disease, e.g., mitigating symptoms associated with the disease. The “treatment” as used herein encompasses administration of a medicament containing the polypeptide or derivative thereof to an individual to treat, cure, alleviate, improve, mitigate, or suppress the individual's disease, including, but not limited to, administrating the medicament containing the polypeptide or the derivative thereof described herein to an individual in need.

The term “non-alcoholic fatty liver disease (NAFLD)” herein usually refers to a clinicopathological syndrome mainly characterized by excessive fat deposition in hepatocytes, excluding those caused by alcohol and other definite liver-damaging factors. It is an acquired metabolic stress-induced liver injury closely related to insulin resistance and genetic susceptibility, including but are not limited to simple fatty liver (SFL), non-alcoholic steatohepatitis (NASH), and their associated cirrhosis.

### Detailed Description of Polypeptide or Analog Thereof of the Present Disclosure and Use Thereof

The present disclosure provides a polypeptide, derivative thereof, pharmaceutically acceptable salt thereof, a pharmaceutical composition, and uses thereof, which will be described in detail below, respectively.

### Polypeptide, Derivative Thereof, or Pharmaceutically Acceptable Salt Thereof

In an aspect of the present disclosure, the present invention proposes a polypeptide, a derivative thereof, or a pharmaceutically acceptable salt thereof. According to an embodiment of the present disclosure, the polypeptide, derivative thereof, or pharmaceutically acceptable salt thereof has a structure represented by formula (I):

U—X₁SHX₄SX₆X₇X₈X₉X₁₀X₁₁X₁₂X₁₃X₁₄X₁₅X₁₆X₁₇X₁₈X₁₉X₂₀DX₂₂PX₂₄TDVGAGSX₃₂—NH₂ (I), wherein:
each U is independently hydrogen, acetyl, or benzoyl;
X₁ is A or K; X₄ is L or αMeL; X₆ is Z₁, Z₂ or T; X₇ is Z₁, Z₂ or A; X₈ is Z₁, Z₂ or V; X₉ is Z₁, Z₂ or L; X₁₀ is Z₁, Z₂ or G; X₁₁ is Z₁, Z₂, homoR, Orn or R; X₁₂ is Z₁, Z₂ or L; X₁₃ is Z₁, Z₂ or S; X₁₄ is Z₁, Z₂ or A; X₁₅ is Z₁, Z₂ or E; X₁₆ is Z₁, Z₂ or L; X₁₇ is Z₁, Z₂ or H; X₁₈ is Z₁, Z₂, K, αMeK or Orn; X₁₉ is Z₁, Z₂ or L; X₂₀ is Z₁, Z₂ or Aib; X₂₂ is Y or αMeF; X₂₄ is R, N-Me-R, homoR, norR, Q or r; and X₃₂ is P, Hyp, cis-P(4-NH₂), trans-P(4-NH₂) or p;
the structure represented by formula (I) includes two amino acids Z₁ or two amino acids Z₂, sites of the two amino acids Z₁ or the two amino acids Z₂ are Xᵢ and Xᵢ₊₇, respectively, where i is any integer between 6 and 13;
each amino acid Z₁ is independently selected from C, c, αMeC, HoC, Hoc, Pen or N-Me-C; and
each amino acid Z₂ is independently selected from K, k, Dap, Dab, Orn, HomoK, N-Me-K, N-Me-k, αMeK or αMek.

The polypeptide, derivative thereof, or pharmaceutically acceptable salt thereof (referred to as a polypeptide or analog thereof) according to the embodiment of the present disclosure is a dual agonist polypeptide analog, which can simultaneously exhibit certain balanced or unbalanced activating activities on both the amylin receptor (AMYR) and the calcitonin receptor (CTR). The polypeptide, derivative thereof, or pharmaceutically acceptable salt thereof can be used to prevent or treat metabolic disorder syndromes, such as T2DM, obesity, hyperlipidemia, NAFLD and NASH; and also has other potential therapeutic applications for diseases, such as Alzheimer's disease (AD), alcohol or drug addiction-related symptoms or diseases, etc.

Further, long in-vivo half-life can be achieved through modification of two amino acids Z₁ or two amino acids Z₂ of the above polypeptide or analog, thereby supporting low-dose and low-frequency administration.

It should be noted that, unless otherwise specified herein, U refers to one hydrogen or a substituent group thereof in the —NH₂ of amino acid X₁. When U is hydrogen, —NH₂ of X₁ is —NH₂ itself; when U is acetyl (i.e., Ac), —NH₂ of X₁ is —NHAc; and when U is benzoyl (i.e., Bzl), —NH₂ of X₁ is —NH(Bzl).

It should be noted that, unless otherwise specified herein, —NH₂ of X₃₂ refers that —OH in —COOH of X₃₂ is substituted with —NH₂, i.e., —CONH₂.

According to an embodiment of the present disclosure, the sites of the two amino acids Z₁ or two amino acids Z₂ are one of the following groups: X₆ and X₁₃, X₇ and X₁₄, X₈ and X₁₅, X₉ and X₁₆, X₁₀ and X₁₇, X₁₁ and X₁₈, X₁₂ and X₁₉, or X₁₃ and X₂₀.

According to an embodiment of the present disclosure, the sites of the two amino acids Z₁ or two amino acids Z₂ are X₈ and X₁₅, respectively.

According to an embodiment of the present disclosure, the two amino acids Z₁ or two amino acids Z₂ are independently selected from K, c or C.

According to an embodiment of the present disclosure, X₁ is A or K.

According to an embodiment of the present disclosure, X₁ is A.

According to an embodiment of the present disclosure, X₄ is L.

According to an embodiment of the present disclosure, X₆ is K, c, C or T.

According to an embodiment of the present disclosure, X₆ is C or T.

According to an embodiment of the present disclosure, X₆ is T.

According to an embodiment of the present disclosure, X₇ is K, c, C or A.

According to an embodiment of the present disclosure, X₇ is C or A.

According to an embodiment of the present disclosure, X₇ is A.

According to an embodiment of the present disclosure, X₈ is C, V, c, HoC, Pen, αMeC, N-Me-C, K, Orn, k or Dab.

According to an embodiment of the present disclosure, X₈ is K, c, C or V.

According to an embodiment of the present disclosure, X₈ is C, V or K

According to an embodiment of the present disclosure, X₈ is C or V.

According to an embodiment of the present disclosure, X₈ is V.

According to an embodiment of the present disclosure, X₉ is K, c, C or L.

According to an embodiment of the present disclosure, X₉ is C or L.

According to an embodiment of the present disclosure, X₉ is L.

According to an embodiment of the present disclosure, X₁₀ is K, c, C or G.

According to an embodiment of the present disclosure, X₁₀ is C or G.

According to an embodiment of the present disclosure, X₁₀ is G.

According to an embodiment of the present disclosure, X₁₁ is K, c, C, homoR, Orn or R.

According to an embodiment of the present disclosure, X₁₁ is K, C, homoR, Orn or R.

According to an embodiment of the present disclosure, X₁₁ is K, C, homoR or R.

According to an embodiment of the present disclosure, X₁₁ is C, homoR or R.

According to an embodiment of the present disclosure, X₁₁ is homoR or R.

According to an embodiment of the present disclosure, X₁₂ is K, c, C or L.

According to an embodiment of the present disclosure, X₁₂ is C or L.

According to an embodiment of the present disclosure, X₁₂ is L.

According to an embodiment of the present disclosure, X₁₃ is K, c, C or S.

According to an embodiment of the present disclosure, X₁₃ is C or S.

According to an embodiment of the present disclosure, X₁₃ is S.

According to an embodiment of the present disclosure, X₁₄ is K, c, C or A.

According to an embodiment of the present disclosure, X₁₄ is C or A.

According to an embodiment of the present disclosure, X₁₄ is A.

According to an embodiment of the present disclosure, X₁₅ is C, E, c, HoC, Pen, αMeC, αMeK, N-Me-C, K, Orn, k or Dab.

According to an embodiment of the present disclosure, X₁₅ is K, c, C or E.

According to an embodiment of the present disclosure, X₁₅ is K, C or E.

According to an embodiment of the present disclosure, X₁₅ is C or E.

According to an embodiment of the present disclosure, X₁₅ is C.

According to an embodiment of the present disclosure, X₁₆ is K, c, C or L.

According to an embodiment of the present disclosure, X₁₆ is C or L.

According to an embodiment of the present disclosure, X₁₆ is L.

According to an embodiment of the present disclosure, X₁₇ is K, c, C or H.

According to an embodiment of the present disclosure, X₁₇ is C or H.

According to an embodiment of the present disclosure, X₁₇ is H.

According to an embodiment of the present disclosure, X₁₈ is c, K, C, k, αMeK or Orn.

According to an embodiment of the present disclosure, X₁₈ is K, C, k, αMeK or Orn.

According to an embodiment of the present disclosure, X₁₈ is C, K or αMeK.

According to an embodiment of the present disclosure, X₁₈ is K or αMeK.

According to an embodiment of the present disclosure, X₁₉ is K, c, C or L.

According to an embodiment of the present disclosure, X₁₉ is C or L.

According to an embodiment of the present disclosure, X₂₁ is L.

According to an embodiment of the present disclosure, X₂₀ is K, c, C or Aib.

According to an embodiment of the present disclosure, X₂₀ is C or Aib.

According to an embodiment of the present disclosure, X₂₀ is Aib.

According to an embodiment of the present disclosure, X₂₂ is Y.

According to an embodiment of the present disclosure, X₂₃ is P.

According to an embodiment of the present disclosure, X₂₄ is R, N-Me-R, homoR, norR, r or Q.

According to an embodiment of the present disclosure, X₂₄ is R, N-Me-R, homoR, norR or Q.

According to an embodiment of the present disclosure, X₂₄ is R, N-Me-R or Q.

According to an embodiment of the present disclosure, X₂₉ is A.

According to an embodiment of the present disclosure, X₃₂ is P or Hyp.

According to an embodiment of the present disclosure, the structure represented by formula (I) has the following structure: U-X₁SHX₄STX₇X₈LGX₁₁LX₁₃X₁₄X₁₅LHX₁₈LX₂₀DX₂₂X₂₃X₂₄TDVGAGSX₃₂—NH₂ (Ia).

According to some optional embodiments of the present disclosure, in the structure represented by formula (Ia), X₁ is A or K.

According to some optional embodiments of the present disclosure, in the structure represented by formula (Ia), X₄ is L or αMeL.

According to some optional embodiments of the present disclosure, in the structure represented by formula (Ia), X₇ is C or A.

According to some optional embodiments of the present disclosure, in the structure represented by formula (Ia), X₈ is C or V.

According to some optional embodiments of the present disclosure, in the structure represented by formula (Ia), X₁₁ is homoR, C or R.

According to some optional embodiments of the present disclosure, in the structure represented by formula (Ia), X₁₃ is C or S.

According to some optional embodiments of the present disclosure, in the structure represented by formula (Ia), X₁₄ is C or A.

According to some optional embodiments of the present disclosure, in the structure represented by formula (Ia), X₁₅ is C or E.

According to some optional embodiments of the present disclosure, in the structure represented by formula (Ia), X₁₈ is C, K, k, αMeK or Orn.

According to some optional embodiments of the present disclosure, in the structure represented by formula (Ia), X₂₀ is C or Aib.

According to some optional embodiments of the present disclosure, in the structure represented by formula (Ia), X₂₂ is Y or αMeL.

According to some optional embodiments of the present disclosure, in the structure represented by formula (Ia), X₂₃ is P or K.

According to some optional embodiments of the present disclosure, in the structure represented by formula (Ia), X₂₄ is R, N-Me-R, homoR, norR or Q.

According to some optional embodiments of the present disclosure, in the structure represented by formula (Ia), X₃₂ is P or Hyp.

According to an embodiment of the present disclosure, the structure represented by formula (I) has the following structure: U—X₁SHX₄STAX₈LGX₁₁LSAX₁₅LHX₁₈L-Aib-DX₂₂PX₂₄TDVGAGSX₃₂—NH₂ (Ib).

According to some optional embodiments of the present disclosure, in a structure represented by formula (Ib), X₁ is A or K.

According to some optional embodiments of the present disclosure, in the structure represented by formula (Ib), X₄ is L or αMeL.

According to some optional embodiments of the present disclosure, in the structure represented by formula (Ib), X₈ is C, V, c, HoC, Pen, αMeC, N-Me-C, K, Orn, k or Dab.

According to some optional embodiments of the present disclosure, in the structure represented by formula (Ib), X₁₁ is homoR, C, K, Orn or R.

According to some optional embodiments of the present disclosure, in the structure represented by formula (Ib), X₁₅ is C, E, c, HoC, Pen, αMeC, αMeK, N-Me-C, K, Orn, k or Dab.

According to some optional embodiments of the present disclosure, in the structure represented by formula (Ib), X₁₈ is C, K, αMeK or Orn.

According to some optional embodiments of the present disclosure, in the structure represented by formula (Ib), X₂₂ is Y or αMeF.

According to some optional embodiments of the present disclosure, in the structure represented by formula (Ib), X₂₄ is R, N-Me-R, homoR, norR, r or Q.

According to some optional embodiments of the present disclosure, in the structure represented by formula (Ib), X₃₂ is P or Hyp.

According to an embodiment of the present disclosure, the structure represented by formula (I) has the following structure: U-ASHX₄STX₇X₈LGX₁₁X₁₂X₁₃X₁₄X₁₅LX₁₇X₁₈X₁₉X₂₀DX₂₂PX₂₄TDVGAGSX₃₂—NH₂ (II).

According to some optional embodiments of the present disclosure, in the structure represented by formula (II), X₄ is L or αMeL.

According to some optional embodiments of the present disclosure, in the structure represented by formula (II), X₇ is C or A.

According to some optional embodiments of the present disclosure, in the structure represented by formula (II), X₈ is C, K or V.

According to some optional embodiments of the present disclosure, in the structure represented by formula (II), X₁₁ is homoR, C, K or R.

According to some optional embodiments of the present disclosure, in the structure represented by formula (II), X₁₂ is L or C.

According to some optional embodiments of the present disclosure, in the structure represented by formula (II), X₁₃ is C or S.

According to some optional embodiments of the present disclosure, in the structure represented by formula (II), X₁₄ is C or A.

According to some optional embodiments of the present disclosure, in the structure represented by formula (II), X₁₅ is K, C or E.

According to some optional embodiments of the present disclosure, in the structure represented by formula (II), X₁₇ is C or H.

According to some optional embodiments of the present disclosure, in the structure represented by formula (II), X₁₈ is C, K, αMeK or Orn.

According to some optional embodiments of the present disclosure, in the structure represented by formula (II), X₁₉ is C or L.

According to some optional embodiments of the present disclosure, in the structure represented by formula (II), X₂₀ is C or Aib.

According to some optional embodiments of the present disclosure, in the structure represented by formula (II), X₂₂ is Y or αMeF.

According to some optional embodiments of the present disclosure, in the structure represented by formula (II), X₂₄ is R, N-Me-R or Q.

According to some optional embodiments of the present disclosure, in the structure represented by formula (II), X₃₂ is P or Hyp.

According to an embodiment of the present disclosure, the structure represented by formula (I) has the following structure: U-ASHX₄STAX₈LGX₁₁LX₁₃AX₁₅LHX₁₈LX₂₀DX₂₂PX₂₄TDVGAGSX₃₂—NH₂ (IIa).

According to some optional embodiments of the present disclosure, in the structure represented by formula (IIa), X₄ is L or αMeL.

According to some optional embodiments of the present disclosure, in the structure represented by formula (IIa), X₈ is C, K or V.

According to some optional embodiments of the present disclosure, in the structure represented by formula (IIa), X₁₁ is homoR, C, K or R.

According to some optional embodiments of the present disclosure, in the structure represented by formula (IIa), X₁₃ is C or S.

According to some optional embodiments of the present disclosure, in the structure represented by formula (IIa), X₁₅ is K, C or E.

According to some optional embodiments of the present disclosure, in the structure represented by formula (IIa), X₁₈ is C, K, αMeK or Orn.

According to some optional embodiments of the present disclosure, in the structure represented by formula (IIa), X₂₀ is C or Aib.

According to some optional embodiments of the present disclosure, in the structure represented by formula (IIa), X₂₂ is Y or αMeL.

According to some optional embodiments of the present disclosure, in the structure represented by formula (IIa), X₂₄ is R, N-Me-R or Q.

According to some optional embodiments of the present disclosure, in the structure represented by formula (IIa), X₃₂ is P or Hyp.

According to an embodiment of the present disclosure, the structure represented by formula (I) has the following structure: U-ASHLSTAX₈LGX₁₁LSAX₁₅LHX₁₈L-Aib-DYPX₂₄TDVGAGSX₃₂—NH₂ (IIb).

According to some optional embodiments of the present disclosure, in the structure represented by formula (IIb), X₈ is C, K or V.

According to some optional embodiments of the present disclosure, in the structure represented by formula (IIb), X₁₁ is homoR, C or R.

According to some optional embodiments of the present disclosure, in the structure represented by formula (IIb), X₁₅ is K, C or E.

According to some optional embodiments of the present disclosure, in the structure represented by formula (IIb), X₁₈ is C, K or αMeK.

According to some optional embodiments of the present disclosure, in the structure represented by formula (IIb), X₂₄ is R, N-Me-R or Q.

According to some optional embodiments of the present disclosure, in the structure represented by formula (IIb), X₃₂ is P or Hyp.

According to an embodiment of the present disclosure, the structure represented by formula (I) has the following structure: U-ASHLSTACLGX₁₁LSACLHX₁₈L-Aib-DYPX₂₄TDVGAGSX₃₂—NH₂ (III).

According to some optional embodiments of the present disclosure, in the structure represented by formula (III), X₁₁ is homoR or R.

According to some optional embodiments of the present disclosure, in the structure represented by formula (III), X₁₈ is K or αMeF.

According to some optional embodiments of the present disclosure, in the structure represented by formula (III), X₂₄ is R, N-Me-R or Q.

According to some optional embodiments of the present disclosure, in the structure represented by formula (III), X₃₂ is P or Hyp.

According to an embodiment of the present disclosure, the structure represented by formula (I) has one of the following structures:
U-ASHLSCAVLGRLCAELHKL-Aib-DYPRTDVGAGSP-NH₂;
U-ASHLSTCVLGRLSCELHKL-Aib-DYPRTDVGAGSP-NH₂;
U-ASHLSTACLGRLSACLHKL-Aib-DYPRTDVGAGSP-NH₂;
U-ASHLSTAVCGRLSAECHKL-Aib-DYPRTDVGAGSP-NH₂;
U-ASHLSTAVLCRLSAELCKL-Aib-DYPRTDVGAGSP-NH₂;
U-ASHLSTAVLGCLSAELHCL-Aib-DYPRTDVGAGSP-NH₂;
U-ASHLSTAVLGRCSAELHKC-Aib-DYPRTDVGAGSP-NH₂;
U-ASHLSTAVLGRLCAELHKLCDYPRTDVGAGSP-NH₂;
U-ASHLSTACLGRLSACLH-Orn-L-Aib-D-(αMeF)-PRTDVGAGS-Hyp-NH₂;
U-ASH-(αMeL)-STACLGRLSACLHKL-Aib-D-(αMeF)—P—(N-Me-R)-TDVGAGSP-NH₂;
U-ASHLSTACLGRLSACLH-(αMeK)-L-Aib-DYP-(N-Me-R)-TDVGAGS-Hyp-NH₂;
U-ASHLSTACLG-homoR-LSACLH-(αMeK)-L-Aib-DYP-(N-Me-R)-TDVGAGSP-NH₂;
U-ASHLSTACLGRLSACLH-(αMeK)-L-Aib-DYP-homoR-TDVGAGS-Hyp-NH₂;
U-ASHLSTACLGRLSACLH-(αMeK)-L-Aib-DYP-norR-TDVGAGS-Hyp-NH₂;
U-ASHLSTACLGRLSACLH-(αMeK)-L-Aib-DYPQTDVGAGS-Hyp-NH₂;
U-ASHLSTACLGRLSACLH-(αMeK)-L-Aib-DYP-r-TDVGAGS-Hyp-NH₂;
U-ASHLSTAVLGCL SAELHCL-Aib-DYPQTDVGAGS-Hyp-NH₂;
U-KSHLSTACLGRLSACLH-(αMeK)-L-Aib-DYP-(N-Me-R)-TDVGAGS-Hyp-NH₂;
U-KSHLSTAVLGCL SAELHCL-Aib-DYPQTDVGAGS-Hyp-NH₂;
U-KSHLSTA-c-LGRLSA-c-LH-(αMeK)-L-Aib-DYPQTDVGAGS-Hyp-NH₂;
U-ASHLSTACLGRLSACLH-(αMeK)-L-Aib-DYPQTDVGAGS-(cis-P(4-NH₂))—NH₂;
U-ASHLSTACLGRLSACLH-(αMeK)-L-Aib-DYPQTDVGAGS-(trans-P(4-NH₂))—NH₂;
U-ASHLSTACLGRLSACLH-(αMeK)-L-Aib-DYPQTDVGAGS-p-NH₂,
U-ASHLSTACLG-homoR-LSACLH-(αMeK)-L-Aib-DYPQTDVGAGS-Hyp-NH₂;
U-ASHLSTACLG-Orn-LSA CLH-(αMeK)-L-Aib-DYPQTDVGAGS-Hyp-NH₂;
U-ASHLSTA-HoC-LGRLSA-HoC-LH-(αMeK)-L-Aib-DYPQTDVGAGS-Hyp-NH₂;
U-ASHLSTA-Pen-LGRLSA-Pen-LH-(αMeK)-L-Aib-DYPQTDVGAGS-Hyp-NH₂;
U-ASHLSTA-(αMeC)-LGRLSA-(αMeC)-LH-(αMeK)-L-Aib-DYPQTDVGAGS-Hyp-NH₂;
U-ASHLSTACLGRLSACLH-(αMeK)-L-Aib-DYPQTDVGAGS-Hyp-NH₂;
U-ASHLSTA-(N-Me-C)-LGRLSA-(N-Me-C)-LH-(αMeK)-L-Aib-DYPQTDVGAGS-Hyp-NH₂;
U-ASHLSTAKLGRLSAKLH-(αMeK)-L-Aib-DYPQTDVGAGS-Hyp-NH₂;
U-ASHLSTAVLGKL SAELHKL-Aib-DYPQTDVGAGS-Hyp-NH₂;
U-ASHLSTA-Orn-LGRLSA-Orn-LH-(αMeK)-L-Aib-DYPQTDVGAGS-Hyp-NH₂;
U-ASHLSTA-Dab-LGRLSA-Dab-LH-(αMeK)-L-Aib-DYPQTDVGAGS-Hyp-NH₂; and
where U is independently selected from H, acetyl or benzoyl.

It should be noted that in the polypeptide having the structure represented by formula (I), the “-” between the amino acids represents an amide bond. Exemplarily, “-” in “L-Aib-D” represents an amide bond.

According to an embodiment of the present disclosure, in the structure represented by formula (I), U is hydrogen.

According to an embodiment of the present disclosure, in the structure represented by formula (I), U is acetyl.

According to an embodiment of the present disclosure, in the structure represented by formula (I), U is benzoyl.

According to an embodiment of the present disclosure, the structure represented by formula (I) has at least one of the following structures in the following table:

| Poly - | | SEQ |
|---|---|---|
| peptide | | ID |
| No. | Amino acid sequence of polypeptide | NO: |
| 1-1 | Ac-ASHLSCAVLGRLCAELHKL-Aib-DYPRTDVGAGSP- | 1 |
| | NH₂ | |
| 1-2 | Ac-ASHLSTCVLGRLSCELHKL-Aib-DYPRTDVGAGSP- | 2 |
| | NH₂ | |
| 1-3 | Ac-ASHLSTACLGRLSACLHKL-Aib-DYPRTDVGAGSP- | 3 |
| | NH₂ | |
| 1-4 | Ac-ASHLSTAVCGRLSAECHKL-Aib-DYPRTDVGAGSP- | 4 |
| | NH₂ | |
| 1-5 | Ac-ASHLSTAVLCRLSAELCKL-Aib-DYPRTDVGAGSP- | 5 |
| | NH₂ | |
| 1-6 | Ac-ASHLSTAVLGCLSAELHCL-Aib-DYPRTDVGAGSP- | 6 |
| | NH₂ | |
| 1-7 | Ac-ASHLSTAVLGRCSAELHKC-Aib-DYPRTDVGAGSP- | 7 |
| | NH₂ | |
| 1-8 | Ac-ASHLSTAVLGRLCAELHKLCDYPRTDVGAGSP-NH₂ | 8 |
| 1-9 | Ac-ASHLSTACLGRLSACLH-Orn-L-Aib-D-(αMeF)- | 9 |
| | PRTDVGAGS-Hyp-NH₂ | |
| 1-10 | Ac-ASH-(αMeL)-STACLGRLSACLHKL-Aib-D- | 10 |
| | (αMeF)-P-(N-Me-R-TDVGAGSP-NH₂ | |
| 1-11 | Ac-ASHLSTACLGRLSACLH-(αMeK)-L-Aib-DYP- | 11 |
| | (N-Me-R)-TDVGAGS-Hyp-NH₂ | |
| 1-12 | Ac-ASHLSTACLG-homoR-LSACLH-(αMeK)-L-Aib-DYP- | 12 |
| | (N-Me-R)-TDVGAGSP-NH₂ | |
| 1-13 | Ac-ASHLSTACLGRLSACLH-(αMeK)-L-Aib-DYP-homoR- | 13 |
| | TDVGAGS-Hyp-NH₂ | |
| 1-14 | Ac-ASHLSTACLGRLSACLH-(αMeK)-L-Aib-DYP-norR- | 14 |
| | TDVGAGS-Hyp-NH₂ | |
| 1-15(AMY-015, | Ac-ASHLSTACLGRLSACLH-(αMeK)-L-Aib- | 15 |
| AMY-016, | DYPQTDVGAGS-Hyp-NH₂ | |
| AMY-017, | | |
| AMY-018) | | |
| 1-19 | Ac-ASHLSTACLGRLSACLH-(αMeK)-L-Aib-DYP-r- | 16 |
| | TDVGAGS-Hyp-NH₂ | |
| 1-20 | Ac-ASHLSTAVLGCLSAELHCL-Aib-DYPQTDVGAGS- | 17 |
| | Hyp-NH₂ | |
| 1-23 | Ac-KSHLSTACLGRLSACLH-(αMeK)-L-Aib-DYP- | 18 |
| | (N-Me-R)-TDVGAGS-Hyp-NH₂ | |
| 1-24 | Ac-KSHLSTAVLGCLSAELHCL-Aib-DYPQTDVGAGS- | 19 |
| | Hyp-NH₂ | |
| 1-25 | Ac-KSHLSTA-c-LGRLSA-c-LH-(αMeK)-L-Aib- | 20 |
| | DYPQTDVGAGS-Hyp-NH₂ | |
| 1-26 | H-ASHLSTACLGRLSACLH-(αMeK)-L-Aib- | 21 |
| | DYPQTDVGAGS-Hyp-NH₂ | |
| 1-27 | Ac-ASHLSTACLGRLSACLH-(αMeK)-L-Aib- | 22 |
| | DYPQTDVGAGS-(cis-P(4-NH₂))-NH₂ | |
| 1-28 | Ac-ASHLSTACLGRLSACLH-(αMeK)-L-Aib- | 23 |
| | DYPQTDVGAGS-(trans-P(4-NH₂))-NH₂ | |
| 1-29 | Ac-ASHLSTACLGRLSACLH-(αMeK)-L-Aib- | 24 |
| | DYPQTDVGAGS-p-NH₂ | |
| 1-30 | Ac-ASHLSTACLG-homoR-LSACLH-(αMeK)- | 25 |
| | L-Aib-DYPQTDVGAGS-Hyp-NH₂ | |
| 1-31 | Ac-ASHLSTACLG-Orn-LSACLH-(αMeK)-L- | 26 |
| | Aib-DYPQTDVGAGS-Hyp-NH₂ | |
| 1-32 | Ac-ASHLSTA-HoC-LGRLSA-HoC-LH-(αMeK)- | 27 |
| | L-Aib-DYPQTDVGAGS-Hyp-NH₂ | |
| 1-33 | Ac-ASHLSTA-Pen-LGRLSA-Pen-LH-(αMeK)- | 28 |
| | L-Aib-DYPQTDVGAGS-Hyp-NH₂ | |
| 1-34 | Ac-ASHLSTA-(αMeC)-LGRLSA-(αMeC)-LH- | 29 |
| | (αMeK)-L-Aib-DYPQTDVGAGS-Hyp-NH₂ | |
| 1-35 | Bzl-ASHLSTACLGRLSACLH-(αMeK)-L-Aib- | 30 |
| | DYPQTDVGAGS-Hyp-NH₂ | |
| 1-36 | Ac-ASHLSTA-(N-Me-C)-LGRLSA-(N-Me-C)- | 31 |
| | LH-(αMeK)-L-Aib-DYPQTDVGAGS-Hyp-NH₂ | |
| 1-21(AMY-021, | Ac-ASHLSTAKLGRLSAKLH-(αMeK)-L-Aib- | 32 |
| AMY-038) | DYPQTDVGAGS-Hyp-NH₂ | |
| 1-22 | Ac-ASHLSTAVLGKLSAELHKL-Aib-DYPQTDVGAGS- | 33 |
| | Hyp-NH₂ | |
| 1-37 | Ac-ASHLSTA-Orn-LGRLSA-Orn-LH-(αMeK)- | 34 |
| | L-Aib-DYPQTDVGAGS-Hyp-NH₂ | |
| 1-39 | Ac-ASHLSTA-Dab-LGRLSA-Dab-LH-(αMeK)- | 35 |
| | L-Aib-DYPQTDVGAGS-Hyp-NH₂ | |
| 1-40 | H-ASHLSTAVLGKLSAELHKL-Aib-DYPQTDVGAGS- | 36 |
| | Hyp-NH₂ | |
| 1-41 | Bzl-ASHLSTAVLGKLSAELHKL-Aib-DYPQTDVGAGS- | 37 |
| | Hyp-NH₂ | |

It should be noted that in the table herein, when a polypeptide codes a plurality of polypeptides in Table A or Table B, that is, the polypeptide in the table has the same main peptide chain as the plurality of polypeptides in Table A or Table B, the only difference being that it does not have a side chain with a modifying group. For example, “1-21 (AMY-021, AMY-038)” refers that the main peptide chain of polypeptide 1-21 in the above table is the same as that of AMY-021 and AMY-038 in Table A, but the polypeptide 1-21 does not contain a side chain with a modifying group; the same is true for “1-15(AMY-015, AMY-016, AMY-017, AMY-018)”.

According to an embodiment of the present disclosure, the polypeptide, derivative thereof, or pharmaceutically acceptable salt thereof further includes a modifying group.

According to an embodiment of the present disclosure, the modifying group is bound to the two amino acids Z₁ or two amino acids Z₂ in the polypeptide represented by formula (I) or the derivative thereof.

According to an embodiment of the present disclosure, the modifying group is bound to —SH on a side chain of the amino acid Z₁ via a sulfur-carbon bond.

According to an embodiment of the present disclosure, the modifying group is bound to —NH₂ on a side chain of the amino acid Z₂ via an amide bond.

According to an optional embodiment of the present disclosure, the modifying group is bound to —SH on a side chain of the amino acid C via a sulfur-carbon bond.

According to an optional embodiment of the present disclosure, the modifying group is bound to ε-amino on a side chain of the amino acid K via an amide bond.

According to an embodiment of the present disclosure, the modifying group has a structure represented by formula (IV).

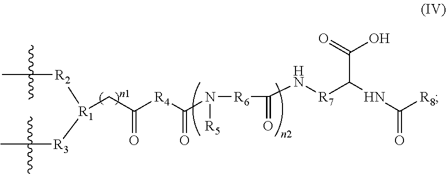

wherein:
R₁ is C, N, —C₃₋₁₀ heteroalkylene, —C₃₋₁₀ arylene or —C₃₋₁₀ heteroarylene;
R₂ and R₃ are each independently —C₁₋₆ alkylene- optionally substituted with one or more R₁ₐ, —NH—C(O)—C₁₋₆ alkylene- optionally substituted with one or more R₁ₐ, or —C₁₋₆ alkylene-NH—C(O)—C₁₋₆ alkylene- optionally substituted with one or more R₁ₐ, wherein each of the one or more R₁ₐ is independently halogen, —OH, —SH, —NH₂, —NO₂, —CN, phenyl, —C₁₋₆ alkyl, —C₁₋₆ halogenated alkyl or —C₁₋₆ alkoxyl;
R₄ is absent, or —C₁₋₆ alkylene-NH—C(O)—C₁₋₆ alkylene- optionally substituted with one or more R₂ₐ, wherein each of the one or more R₂ₐ is independently halogen, —OH, —SH, —NH₂, —NO₂, —CN, phenyl, —C₁₋₆ alkyl, —C₁₋₆ halogenated alkyl or —C₁₋₆ alkoxyl;
R₅ is H, —C₁₋₆ alkoxyl or —C₁₋₆ alkyl, wherein the —C₁₋₆ alkyl and the —C₁₋₆ alkoxyl are each independently and optionally substituted with one or more halogens, —OH, —C(O)OH, —C(O)—, —SH, —NH₂, —NO₂ and —CN;
R₆ is —C₁₋₆ alkylene- optionally substituted with one or more R₃ₐ, or —(C₁₋₃ alkylene-O)ₘ₁—C₁₋₆ alkylene- optionally substituted with one or more R₃ₐ, wherein each of the one or more R₃ₐ is independently halogen, —OH, —SH, —NH₂, —NO₂, —CN, phenyl, —C₁₋₆ alkyl, —C₁₋₆ halogenated alkyl or —C₁₋₆ alkoxyl;
R₇ is —C₁₋₆ alkylene- optionally substituted with one or more R₄ₐ, wherein each of the one or more R₄ₐ is independently halogen, —OH, —SH, —NH₂, —NO₂, —CN, phenyl, —C₁₋₆ alkyl, —C₁₋₆ halogenated alkyl or —C₁₋₆ alkoxyl;
R₈ is —C₁₀₋₂₀ alkyl optionally substituted with one or more R₅ₐ, —C₁₀₋₂₀ alkylene-R₉ optionally substituted with one or more R₅ₐ, —C₅₋₁₀ alkylene-O—C₃₋₁₀ arylene-R₉ optionally substituted with one or more R₅ₐ, or —C₅₋₁₀ alkylene-O—C₃₋₁₀ heteroarylene-R₉ optionally substituted with one or more R₅ₐ, wherein each of the one or more R₅ₐ is independently halogen, —OH, —SH, —NH₂, —NO₂, —CN, phenyl, —C₁₋₆ alkyl, —C₁₋₆ halogenated alkyl or —C₁₋₆ alkoxyl;
R₉ is —COOH, —C₃₋₇ heteroaryl, —S(O)₂OH or —PO(OH)₂;
m1 is any integer from 1 to 6; and
n1 is any integer from 0 to 6; and
n2 is any integer from 1 to 10.

It should be noted that, the “-” in the modifying group in the present disclosure represents a chemical bond connecting the chemical groups, such as a covalent bond between atoms (or atoms in the groups).

According to an embodiment of the present disclosure, R₁ is C, N, —C₅₋₇ heteroalkylene, —C₅₋₇ arylene or —C₅₋₇ heteroarylene.

According to an embodiment of the present disclosure, R₁ is N, phenylene or pyridylene.

According to an embodiment of the present disclosure, R₁ is

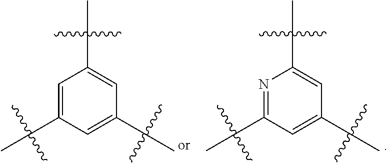 [SMILES: *c1cc(*)cc(*)c1 ; *c1cc(*)nc(*)c1]

According to an embodiment of the present disclosure, R₂ and R₃ are each independently —C₁₋₆ alkylene- or —C₁₋₆ alkylene-NH—C(O)—C₁₋₆ alkylene-.

According to an embodiment of the present disclosure, R₂ and R₃ are each independently —C₁₋₃ alkylene- or —C₁₋₃ alkylene-NH—C(O)—C₁₋₃ alkylene-.

According to an embodiment of the present disclosure, each n1 is independently 0, 1, 2, 3, 4 or 5.

According to an embodiment of the present disclosure, n1 is 0.

According to an embodiment of the present disclosure, R₄ is absent, or —C₁₋₆ alkylene-NH—C(O)—C₁₋₆ alkylene-.

According to an embodiment of the present disclosure, R₄ is absent, or —C₁₋₃ alkylene-NH—C(O)—C₁₋₃ alkylene-.

According to an embodiment of the present disclosure, R₅ is H, —C₁₋₃ alkoxyl or —C₁₋₃ alkyl.

According to an embodiment of the present disclosure, R₆ is —C₁₋₆ alkylene- or —(C₁₋₃ alkylene-O)ₘ₁—C₁₋₆ alkylene-.

According to an embodiment of the present disclosure, R₆ is —C₁₋₃ alkylene- or —(C₁₋₃ alkylene-O)ₘ₁—C₁₋₃ alkylene-.

According to an embodiment of the present disclosure, m1 is 2, 3, 4 or 5.

According to an embodiment of the present disclosure, R₇ is —C₁₋₆ alkylene-.

According to an embodiment of the present disclosure, R₇ is —C₂₋₄ alkylene-.

According to an embodiment of the present disclosure, R₈ is —C₁₀₋₂₀ alkyl, —C₁₀₋₂₀ alkylene-R₉, —C₅₋₁₀ alkylene-O—C₃₋₁₀ arylene-R₉, or —C₅₋₁₀ alkylene-O—C₃₋₁₀ heteroarylene-R₉.

According to an embodiment of the present disclosure, R₈ is —C₁₀₋₁₈ alkyl, —C₁₄₋₁₈ alkylene-R₉ or —C₇₋₉ alkylene-O—C₅₋₇ arylene-COOH.

According to an embodiment of the present disclosure, R₉ is —COOH, —C₅₋₆ heteroaryl, —S(O)₂OH or —PO(OH)₂.

According to an embodiment of the present disclosure, R₉ is —COOH, —S(O)₂OH, —PO(OH)₂ or

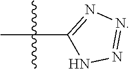 [SMILES: *c1nnn[nH]1]

According to an embodiment of the present disclosure, n2 is 1, 2 or 3.

According to an embodiment of the present disclosure, n2 is 3, 4, 5 or 6.

According to an embodiment of the present disclosure, the modifying group represented by formula (IV) has a structure represented by formula (IVa):

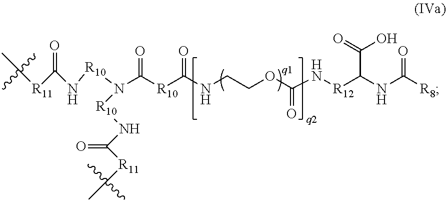

wherein:
each R₁₀ and each R₁₁ are independently —C₀₋₃ alkylene-;
R₁₂ is —C₁₋₆ alkylene-;
R₈ is —C₁₀₋₂₁ alkyl or —C₁₀₋₂₁ alkylene-COOH, and preferably —C₁₄₋₁₉ alkyl or —C₁₀₋₁₈ alkylene-COOH;
q1 is 1, 2, 3 or 4; and
q2 is 1, 2 or 3.

According to an embodiment of the present disclosure, the modifying group represented by formula (IVa) has a structure represented by one of the following formulas:

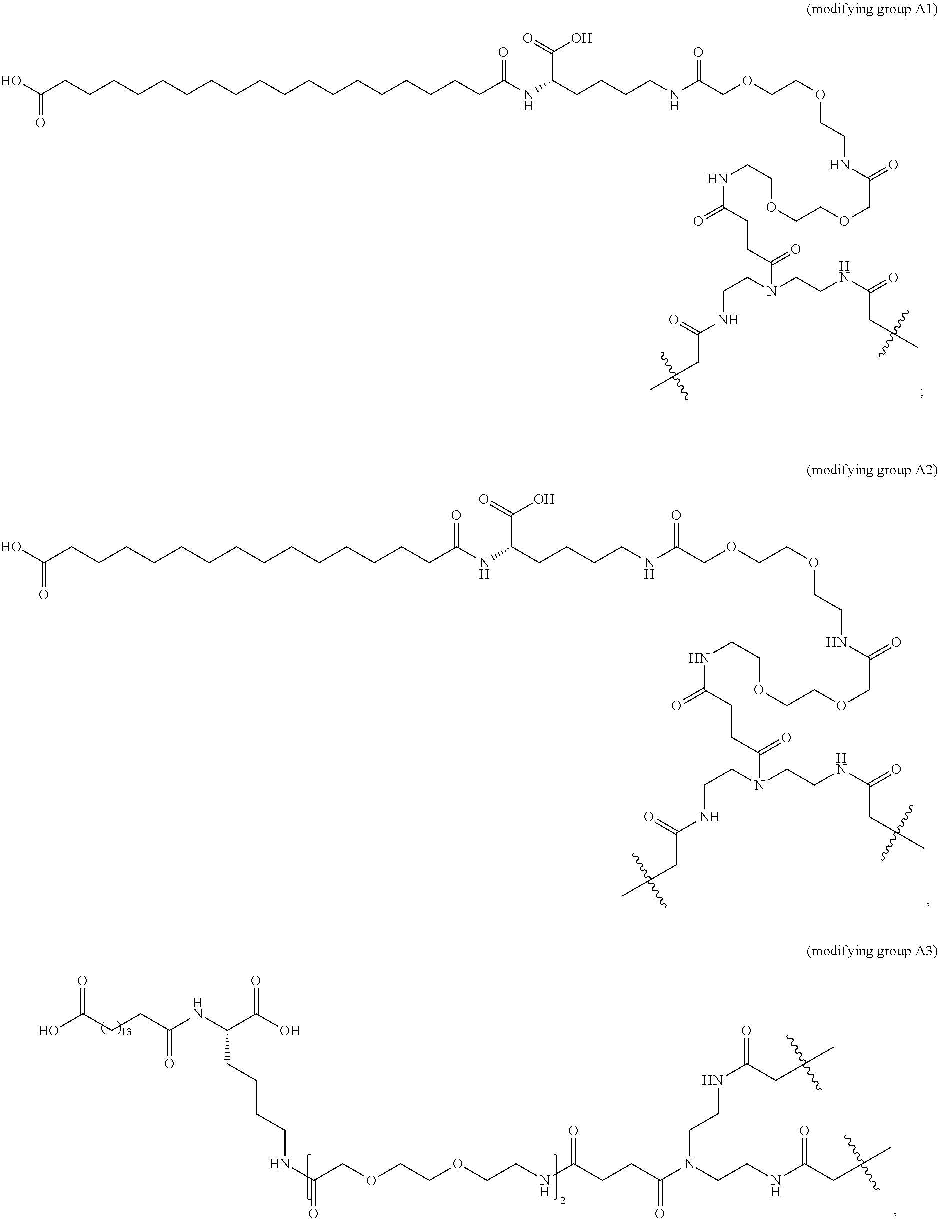

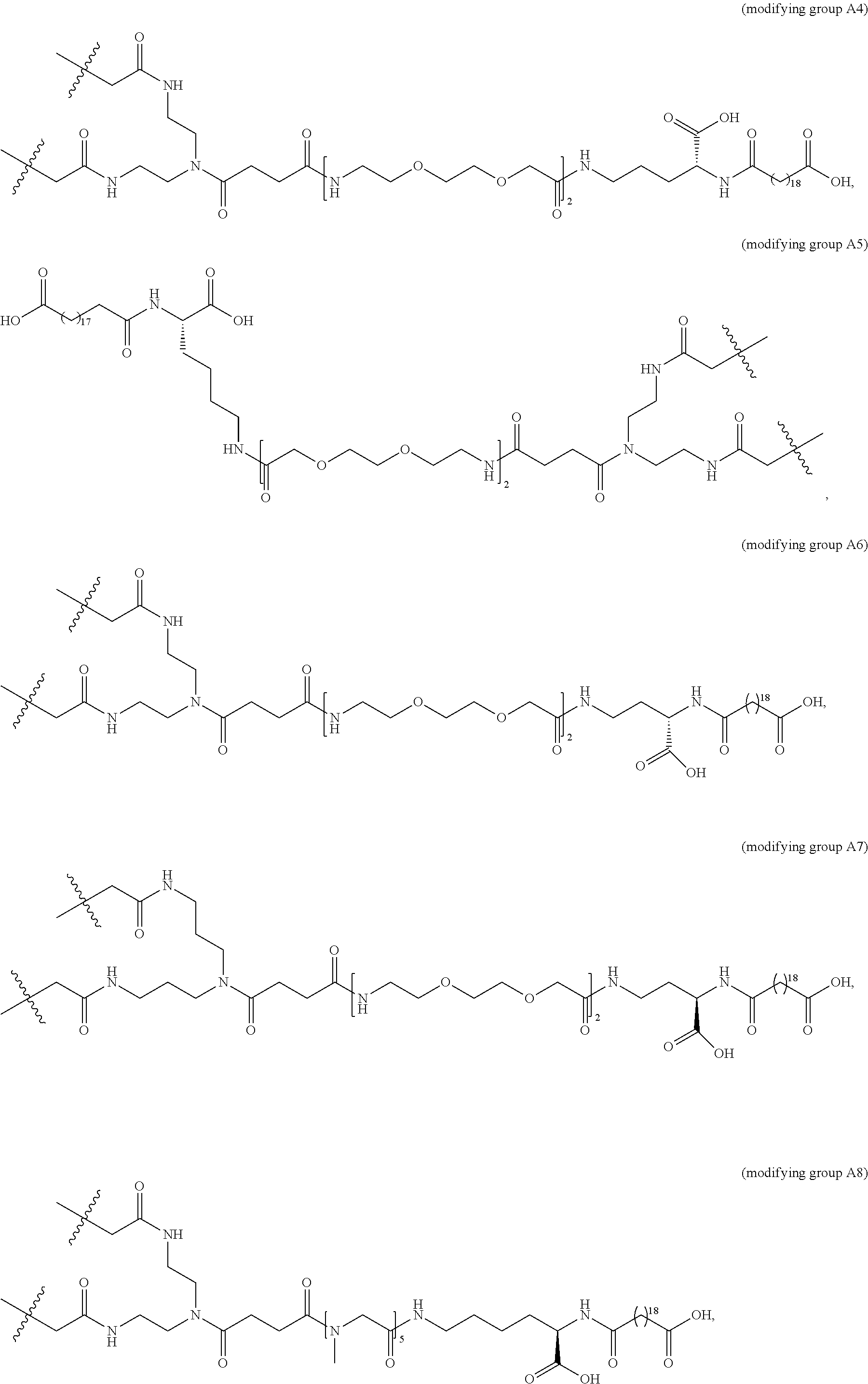

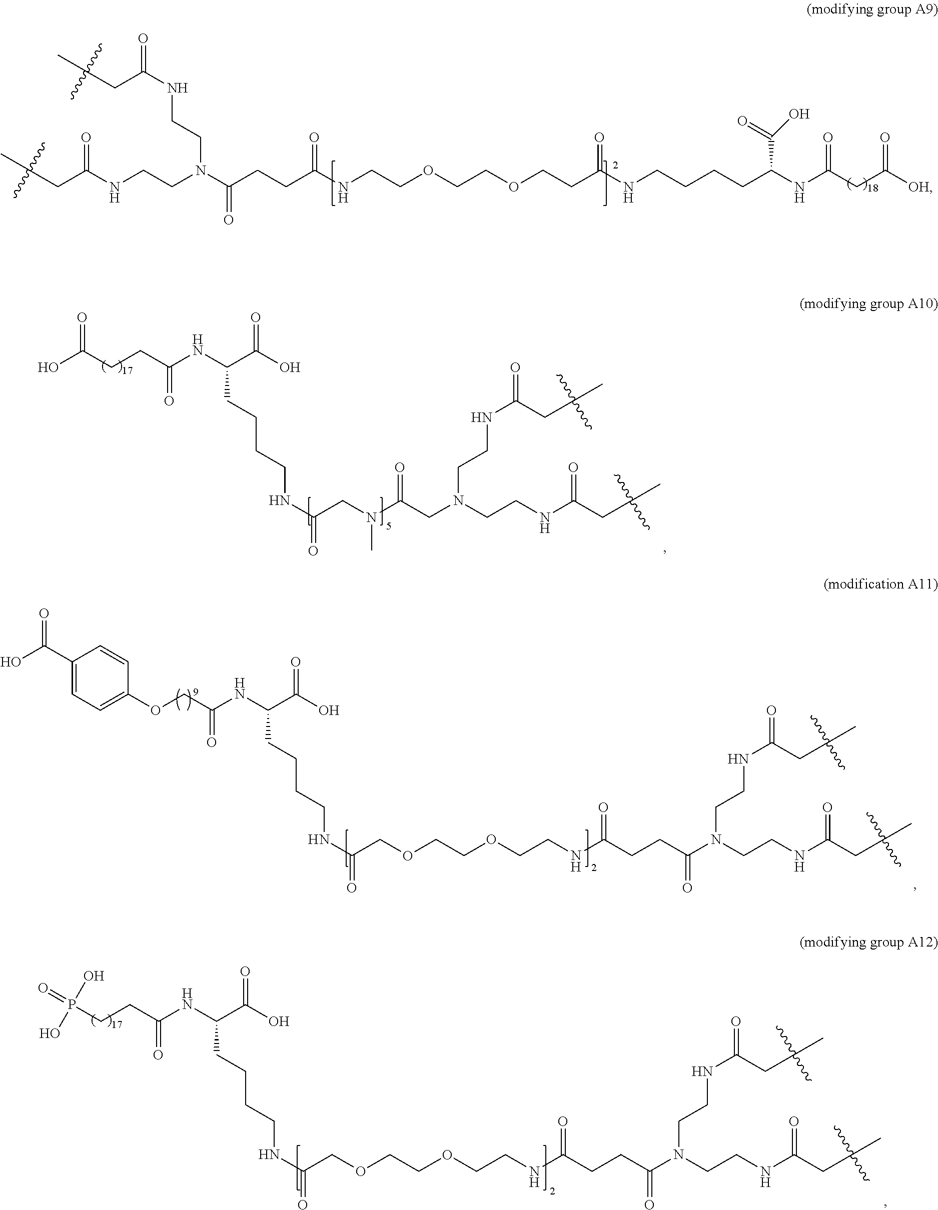

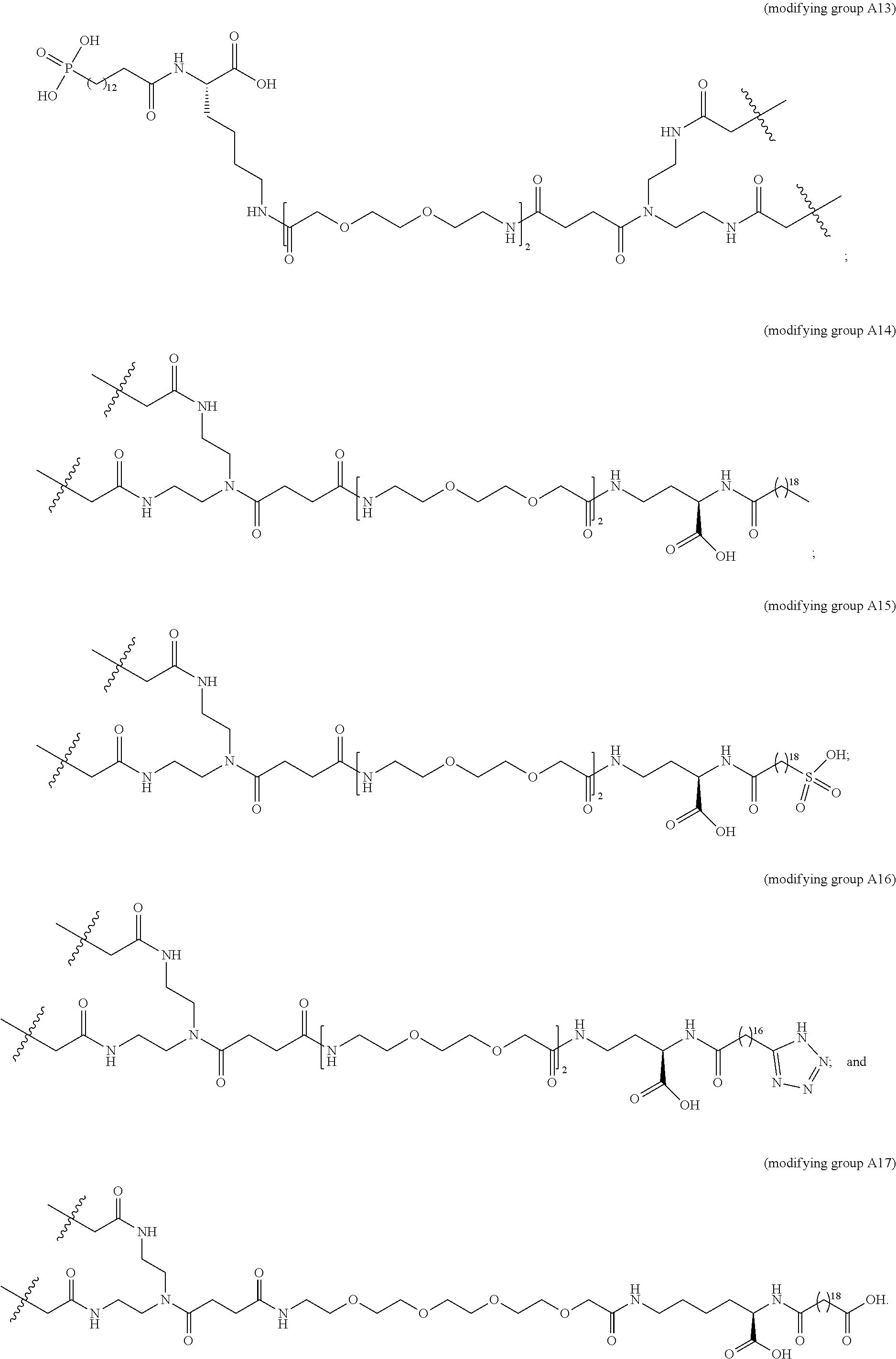

According to an embodiment of the present disclosure, the modifying group represented by formula (IV) has a structure represented by formula (IVb):

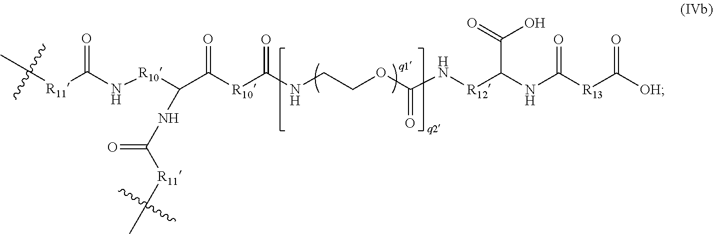

wherein:
each R₁₀′ and each R₁₁′ are independently —C₀₋₃ alkylene-;
R₁₂′ is —C₁₋₆ alkylene-;
R₁₃ is —C₁₀₋₂₀ alkylene-, and preferably —C₁₄₋₁₈ alkylene-;
q1′ is 1, 2, 3 or 4; and
q2′ is 1, 2 or 3.

According to an embodiment of the present disclosure, the modifying group represented by formula (IVb) has a structure represented by one of the following formulas:

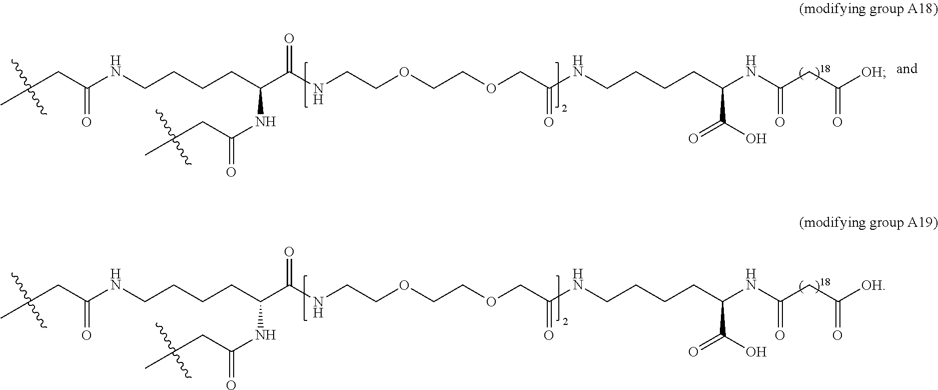

According to an embodiment of the present disclosure, the modifying group represented by formula (IV) has a structure represented by formula (IVc):

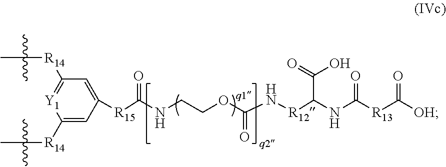

wherein:
each R₁₄ is independently —C₁₋₃ alkylene- or —NH—C(O)—C₁₋₃ alkylene-;
R₁₅ is —C₀₋₃ alkylene- or —C(O)—NH—C₁₋₃ alkylene-;
R₁₂″ is —C₁₋₆ alkylene-;
R₁₃′ is —C₁₀₋₂₀ alkylene-, and preferably —C₁₄₋₁₈ alkylene-;
q1″ is 1, 2, 3 or 4;
q2″ is 1, 2 or 3; and
Y₁ is C or N.

According to an embodiment of the present disclosure, the modifying group represented by formula (IVc) has a structure represented by one of the following formulas:

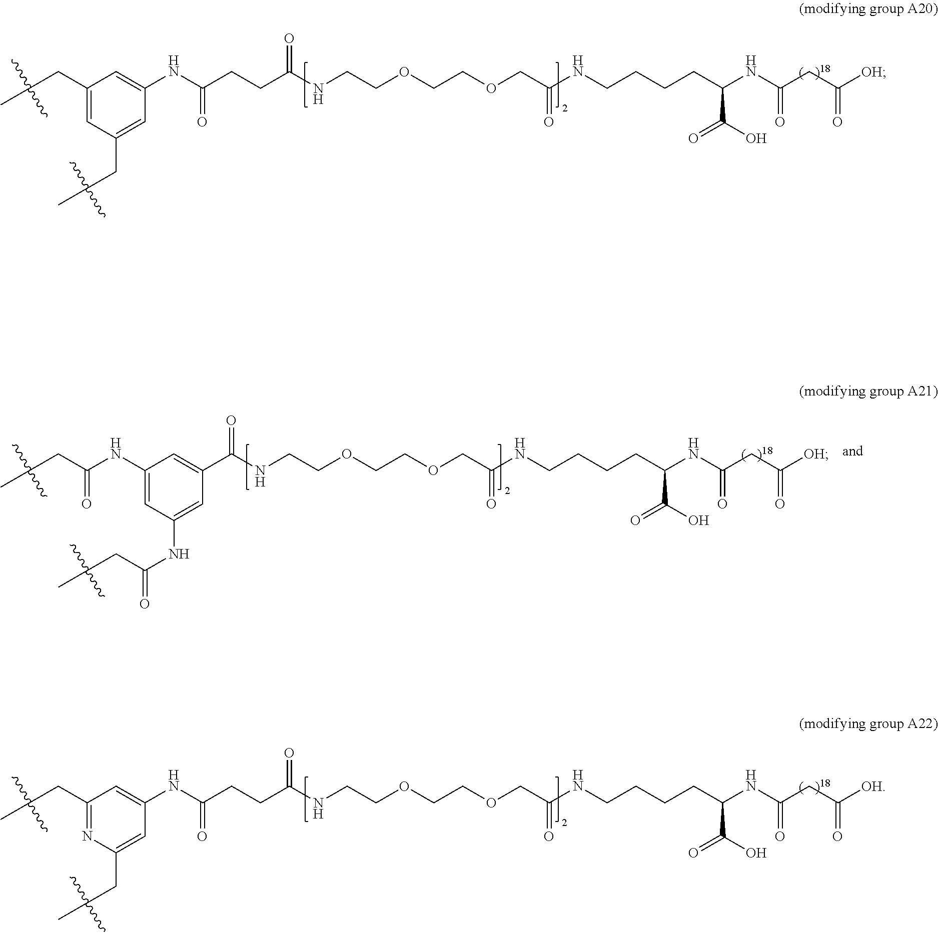

According to an embodiment of the present disclosure, the polypeptide represented by formula (I) or the derivative thereof includes the two amino acids Z₁, and the modifying group has a structure represented by formula (IV).

According to an embodiment of the present disclosure, the modifying group has a structure represented by formula (V):

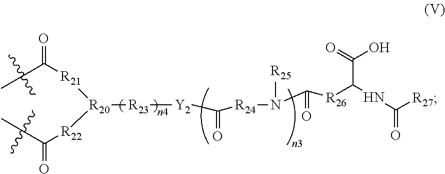

wherein:
R₂₀ is C, N, —C₃₋₁₀ arylene or —C₃₋₁₀ heteroarylene;
R₂₁ and R₂₂ are each independently absent, or —C₁₋₆ alkylene- optionally substituted with one or more R_{1b}, or —C₁₋₆ alkoxylene- optionally substituted with one or more R_{1b}, wherein each of the one or more R_{1b} is independently halogen, —OH, —SH, —NH₂, —NO₂, —CN, phenyl, —C₁₋₆ alkyl, —C₁₋₆ halogenated alkyl or —C₁₋₆ alkoxyl;
R₂₃ is absent, —C₁₋₆ alkylene- optionally substituted with one or more R_{2b}, —C(O)—C₁₋₆ alkylene- optionally substituted with one or more R_{2b}, —NH—C(O)—C₁₋₆ alkylene- optionally substituted with one or more R_{2b}, or —C₁₋₆ alkylene-C(O)—NH—C₁₋₆ alkylene- optionally substituted with one or more R_{2b}, wherein each of the one or more R_{2b} is independently halogen, —OH, —SH, —NH₂, —NO₂, —CN, phenyl, —C₁₋₆ alkyl, —C₁₋₆ halogenated alkyl or —C₁₋₆ alkoxyl;
R₂₄ is —C₁₋₆ alkylene- optionally substituted with one or more R_{3b}, or —(C₁₋₃ alkylene-O)ₘ₂—C₁₋₆ alkylene- optionally substituted with one or more R_{3b}, wherein each of the one or more R_{3b} is independently halogen, —OH, —SH, —NH₂, —NO₂, —CN, phenyl, —C₁₋₆ alkyl, —C₁₋₆ halogenated alkyl or —C₁₋₆ alkoxyl;
R₂₅ is H, —C₁₋₆ alkoxyl or —C₁₋₆ alkyl, wherein the —C₁₋₆ alkyl and the —C₁₋₆ alkoxyl are each independently and optionally substituted with one or more halogens, —OH, —C(O)OH, —C(O)—, —SH, —NH₂, —NO₂ or —CN;
R₂₆ is —C₁₋₆ alkylene- optionally substituted with one or more R_{4b}, wherein each of the one or more R_{4b} is independently halogen, —OH, —SH, —NH₂, —NO₂, —CN, phenyl, —C₁₋₆ alkyl, —C₁₋₆ halogenated alkyl or —C₁₋₆ alkoxyl;
R₂₇ is —C₁₀₋₂₀ alkyl optionally substituted with one or more R_{5b}, —C₁₀₋₂₀ alkylene-R₂₈ optionally substituted with one or more R_{5b}, —C₅₋₁₀ alkylene-O—C₃₋₁₀ arylene-R₂₈ optionally substituted with one or more R_{5b}, or —C₅₋₁₀ alkylene-O—C₃₋₁₀ heteroarylene-R₂₈ optionally substituted with one or more R_{5b}, wherein each of the one or more R_{5b} is independently halogen, —OH, —SH, —NH₂, —NO₂, —CN, phenyl, —C₁₋₆ alkyl, —C₁₋₆ halogenated alkyl or —C₁₋₆ alkoxyl;
R₂₈ is —COOH, —C₃₋₇ heteroaryl, —S(O)₂OH or —PO(OH)₂;
m2 is any integer from 1 to 6; and
n3 is any integer from 1 to 10; and
n3 is 0, 1 or 2; and
Y₂ is absent or NH.

According to an embodiment of the present disclosure, R₂₀ is C, N, —C₅₋₇ arylene or —C₅₋₇ heteroarylene.

According to an embodiment of the present disclosure, R₂₀ is C, N, phenylene or pyridylene.

According to an embodiment of the present disclosure, R₂₀ is

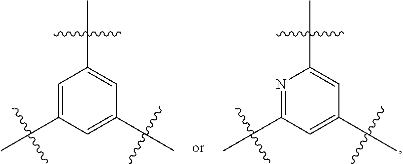 [SMILES: *c1cc(*)cc(*)c1 ; *c1cc(*)nc(*)c1]

and preferably

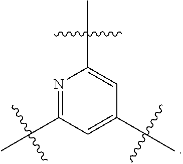 [SMILES: *c1cc(*)nc(*)c1]

According to an embodiment of the present disclosure, R₂₁ and R₂₂ are each independently absent, —C₁₋₆ alkylene- or —C₁₋₆ alkoxylene-.

According to an embodiment of the present disclosure, R₂₁ and R₂₂ are each independently absent or —C₁₋₃ alkylene-.

According to an embodiment of the present disclosure, R₂₃ is absent, —C₁₋₆ alkylene-, —C₁₋₆ alkylene-C(O)—NH—C₁₋₆ alkylene- or —C(O)—C₁₋₆ alkylene-.

According to an embodiment of the present disclosure, R₂₃ is absent, —C₁₋₃ alkylene- or —C(O)—C₂₋₄ alkylene-.

According to an embodiment of the present disclosure, R₂₄ is —C₁₋₆ alkylene- or —(C₁₋₃ alkylene-O)ₘ₂—C₁₋₆ alkylene-.

According to an embodiment of the present disclosure, R₂₄ is —C₁₋₃ alkylene- or —(C₁₋₃ alkylene-O)ₘ₂—C₁₋₃ alkylene-.

According to an embodiment of the present disclosure, m2 is 2, 3, 4 or 5.

According to an embodiment of the present disclosure, R₂₅ is H, —C₁₋₆ alkoxyl or —C₁₋₆ alkyl.

According to an embodiment of the present disclosure, R₂₅ is H or —C₁₋₃ alkyl.

According to an embodiment of the present disclosure, R₂₆ is —C₁₋₆ alkylene-.

According to an embodiment of the present disclosure, R₂₆ is —C₁₋₃ alkylene-.

According to an embodiment of the present disclosure, R₂₇ is —C₁₀₋₂₀ alkyl, —C₁₀₋₂₀ alkylene-R₂₈, —C₅₋₁₀ alkylene-O—C₃₋₁₀ arylene-R₂₈, or —C₅₋₁₀ alkylene-O—C₃₋₁₀ heteroarylene-R₂₈.

According to an embodiment of the present disclosure, R₂₇ is —C₁₀₋₁₈ alkyl, —C₁₄₋₁₈ alkylene-R₂₈ or —C₇₋₉ alkylene-O—C₅₋₇ arylene-COOH.

According to an embodiment of the present disclosure, R₂₈ is —COOH, —C₅₋₆ heteroaryl, —S(O)₂OH or —PO(OH)₂.

According to an embodiment of the present disclosure, R₂₈ is —COOH, —S(O)₂OH, —PO(OH)₂ or

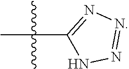 [SMILES: *c1nnn[nH]1]

According to an embodiment of the present disclosure, p is 1, 2 or 3.

According to an embodiment of the present disclosure, p is 3, 4, 5 or 6.

According to an embodiment of the present disclosure, the modifying group represented by formula (V) has a structure represented by formula (Va):

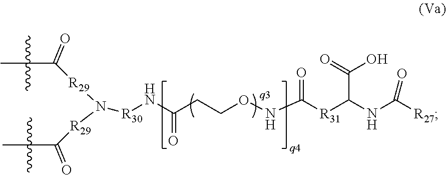

wherein:
each R₂₉ is independently —C₁₋₃ alkylene-;
R₃₀ is —C(O)—C₁₋₆ alkylene-, or —C₁₋₃ alkylene-C(O)—NH—C₁₋₃ alkylene-;
R₃₁ is —C₁₋₆ alkylene-;
q3 is 1, 2, 3 or 4; and
q4 is 1, 2 or 3.

According to an embodiment of the present disclosure, in the formula (Va), R₂₇ is —C₁₀₋₂₀ alkylene-COOH or —C₁₀₋₂₀ alkyl, and preferably —C₁₄₋₁₈ alkylene-COOH or —C₁₀₋₁₉ alkyl.

According to an embodiment of the present disclosure, the modifying group represented by formula (Va) has a structure represented by one of the following formulas:

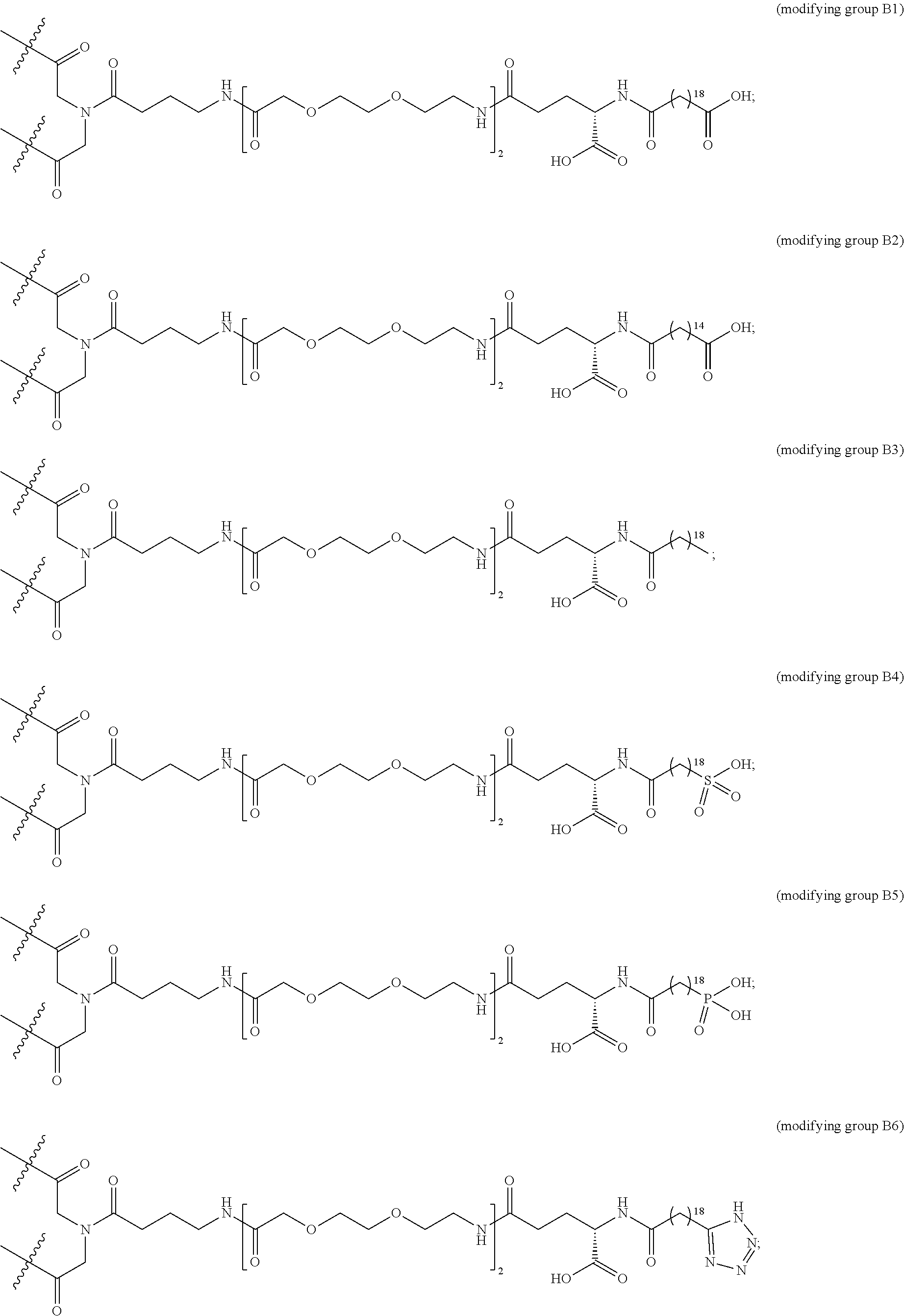

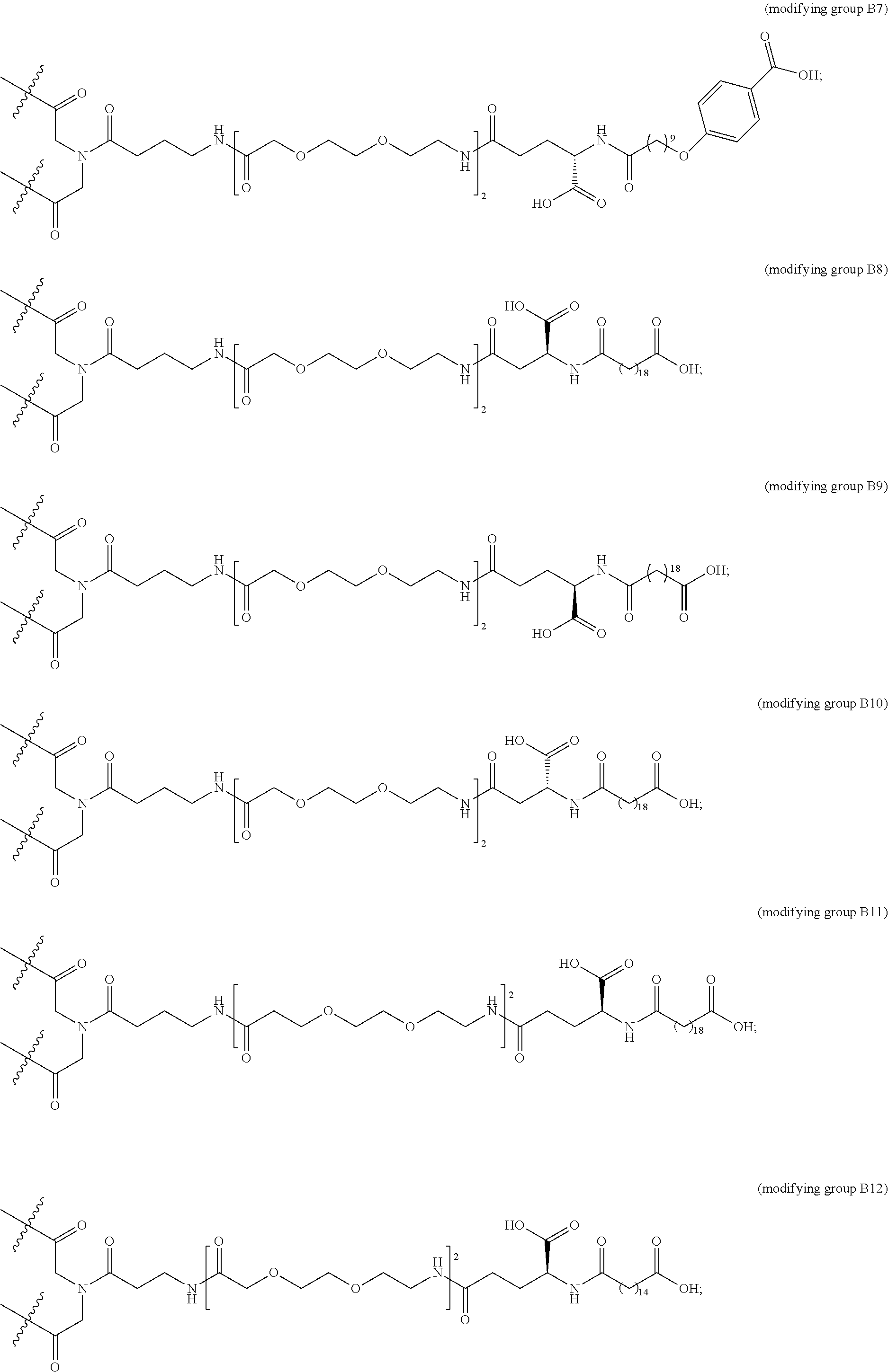

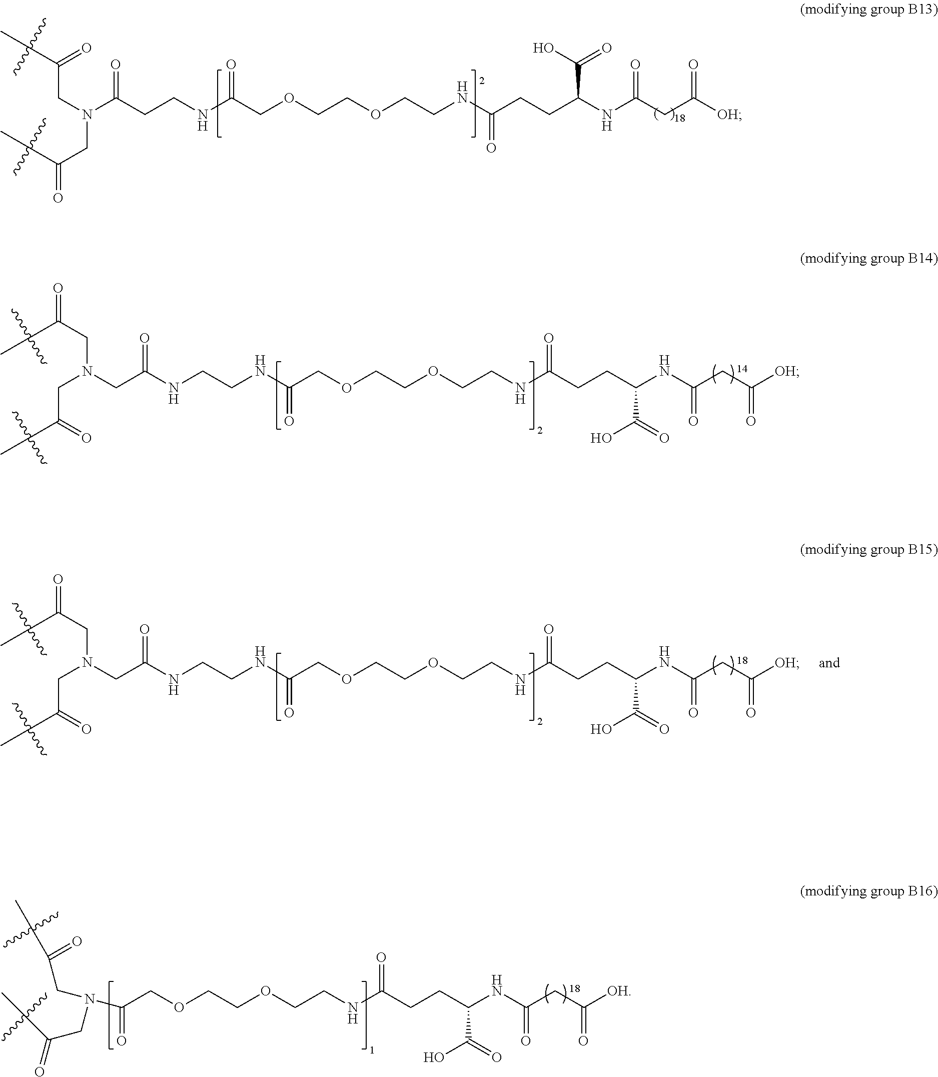

According to an embodiment of the present disclosure, the modifying group represented by formula (V) has a structure represented by formula (Vb):

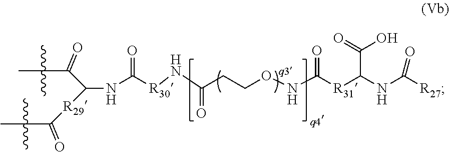

wherein:
each R₂₉′ is independently —C₁₋₃ alkylene-;
R₃₀′ is —C₁₋₆ alkylene-;
R₃₁′ is —C₁₋₆ alkylene-;
q3′ is 1, 2, 3 or 4, and preferably 2; and
q4′ is 1, 2 or 3, and preferably 2.

According to an embodiment of the present disclosure, in the formula (Vb), R₂₇ is —C₁₀₋₂₀ alkylene-COOH or —C₁₀₋₂₀ alkyl, and preferably —C₁₄₋₁₈ alkylene-COOH.

According to an embodiment of the present disclosure, the modifying group represented by formula (Vb) has a structure represented by one of the following formulas:

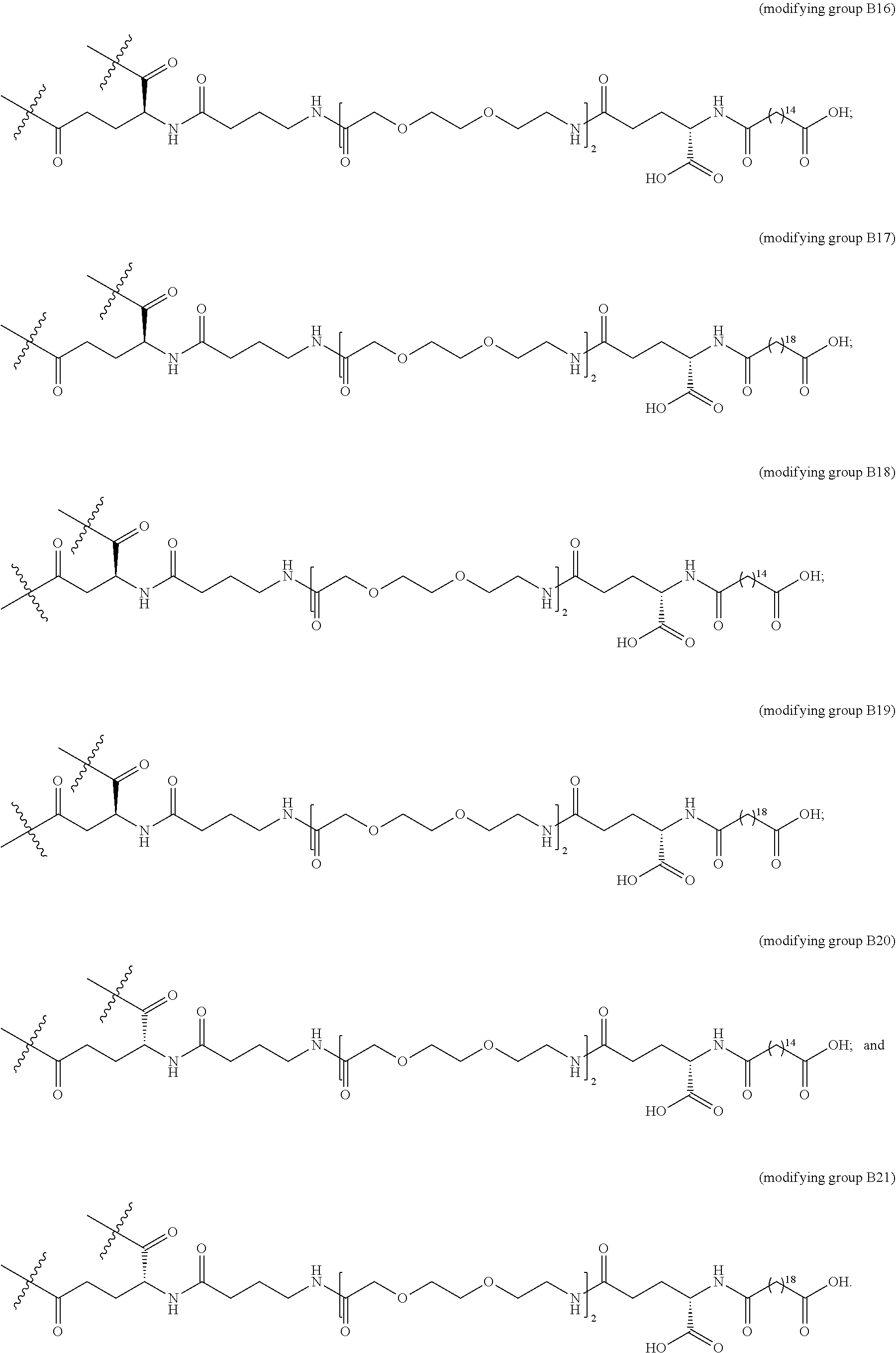

According to an embodiment of the present disclosure, the modifying group represented by formula (V) has a structure represented by formula (Vc):

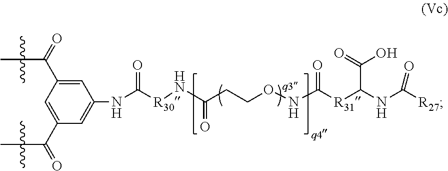

wherein:
R₃₀″ is —C₁₋₆ alkylene-;
R₃₁″ is —C₁₋₆ alkylene-;
q3 is 1, 2, 3 or 4; and
q4 is 1, 2 or 3.

According to an embodiment of the present disclosure, in the formula (Vc), R₂₇ is —C₁₀₋₂₀ alkylene-COOH or —C₁₀₋₂₀ alkyl, and preferably —C₁₄₋₁₈ alkylene-COOH or —C₁₀₋₁₉ alkyl.

According to an embodiment of the present disclosure, the modifying group represented by formula (Vc) has a structure represented by one of the following formulas:

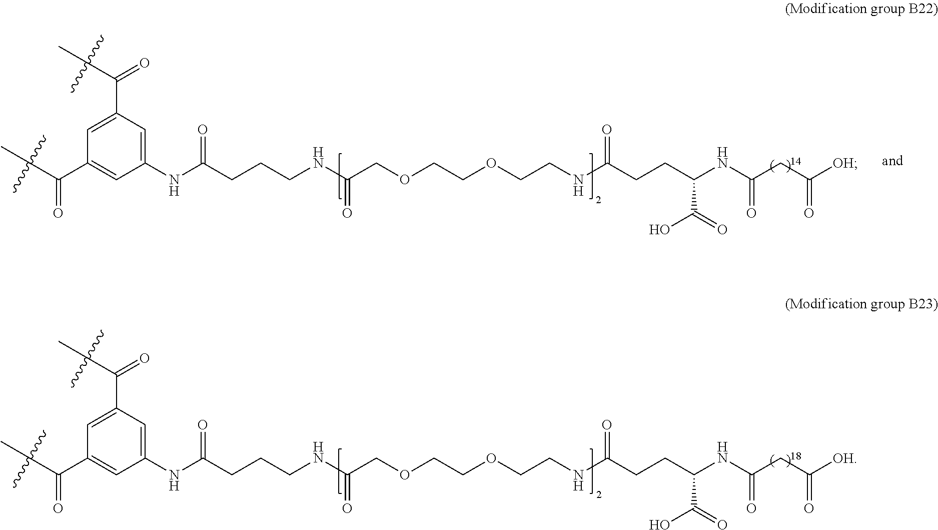

According to an embodiment of the present disclosure, the polypeptide represented by formula (I) or the derivative thereof includes the two amino acids Z₂, and the modifying group has a structure represented by formula (V).

According to an embodiment of the present disclosure, the polypeptide derivative has a structure as shown in any one of the following tables:

| | | Binding sites | |
|---|---|---|---|
| Poly- | | (i, i + 7) of | |
| peptide | Amino acid sequence of | modifying | Modifying |
| No. | polypeptide | groups | group |
| 2-1 | U-ASHLSCAVLGRLCAELHKL-Aib- | 6, 13 | A1 |
| | DYPRTDVGAGSP-NH₂ | | |
| 2-2 | U-ASHLSTCVLGRLSCELHKL-Aib- | 7, 14 | A1 |
| | DYPRTDVGAGSP-NH₂ | | |
| 2-3 | U-ASHLSTACLGRLSACLHKL-Aib- | 8, 15 | A1 |
| | DYPRTDVGAGSP-NH₂ | | |
| 2-4 | U-ASHLSTAVCGRLSAECHKL-Aib- | 9, 16 | A1 |
| | DYPRTDVGAGSP-NH₂ | | |
| 2-5 | U-ASHLSTAVLCRLSAELCKL-Aib- | 10, 17 | A1 |
| | DYPRTDVGAGSP-NH₂ | | |
| 2-6 | U-ASHLSTAVLGCLSAELHCL-Aib- | 11, 18 | A1 |
| | DYPRTDVGAGSP-NH₂ | | |
| 2-7 | U-ASHLSTAVLGRCSAELHKC-Aib- | 12, 19 | A1 |
| | DYPRTDVGAGSP-NH₂ | | |
| 2-8 | U- | 13, 20 | A1 |
| | ASHLSTAVLGRLCAELHKLCDYPRTDV | | |
| | GAGSP-NH₂ | | |
| 2-9 | U-ASHLSTACLGRLSACLH-Orn-L-Aib- | 8, 15 | A1 |
| | D-(αMeF)-PRTDVGAGS-Hyp-NH₂ | | |
| 2-10 | U-ASH-(αMeL)-STACLGRLSACLHKL- | 8, 15 | A1 |
| | Aib-D-(αMeF)-P-(N-Me-R)-TDVGAGSP- | | |
| | NH₂ | | |
| 2-11 | U-ASHLSTACLGRLSACLH-(αMeK)-L- | 8, 15 | A1 |
| | Aib-DYP-(N-Me-R)-TDVGAGS-Hyp-NH₂ | | |
| 2-12 | U-ASHLSTACLG-homoR-LSACLH- | 8, 15 | A1 |
| | (αMeK)-L-Aib-DYP-(N-Me-R)- | | |
| | TDVGAGSP-NH₂ | | |
| 2-13 | U-ASHLSTACLGRLSACLH-(αMeK)-L- | 8, 15 | A1 |
| | Aib-DYP-homoR-TDVGAGS-Hyp-NH₂ | | |
| 2-14 | U-ASHLSTACLGRLSACLH-(αMeK)-L- | 8, 15 | A1 |
| | Aib-DYP-norR-TDVGAGS-Hyp-NH₂ | | |
| 2-15(AMY- | U-ASHLSTACLGRLSACLH-(αMeK)-L- | 8,15 | A1 |
| 015, AMY- | Aib-DYPQTDVGAGS-Hyp-NH₂ | | |
| 035) | | | |
| 2-16(AMY- | U-ASHLSTACLGRLSACLH-(αMeK)-L- | 8,15 | A12 |
| 016, AMY- | Aib-DYPQTDVGAGS-Hyp-NH₂ | | |
| 026) | | | |
| 2-17 | U-ASHLSTACLGRLSACLH-(αMeK)-L- | 8,15 | A5 |
| | Aib-DYPQTDVGAGS-Hyp-NH₂ | | |
| 2-18 | U-ASHLSTACLGRLSACLH-(αMeK)-L- | 8,15 | A4 |
| | Aib-DYPQTDVGAGS-Hyp-NH₂ | | |
| 2-19 | U-ASHLSTACLGRLSACLH-(αMeK)-L- | 8,15 | A1 |
| | Aib-DYP-r-TDVGAGS-Hyp-NH₂ | | |
| 2-20 | U-ASHLSTAVLGCLSAELHCL-Aib- | 11, 18 | A1 |
| | DYPQTDVGAGS-Hyp-NH₂ | | |
| 2-23 | U-KSHLSTACLGRLSACLH-(αMeK)-L- | 8, 15 | A1 |
| | Aib-DYP-(N-Me-R)-TDVGAGS-Hyp-NH₂ | | |
| 2-24 | U-KSHLSTAVLGCLSAELHCL-Aib- | 11, 18 | A1 |
| | DYPQTDVGAGS-Hyp-NH₂ | | |
| 2-25 | U-KSHLSTA-c-LGRLSA-c-LH-(αMeK)-L- | 8,15 | A1 |
| | Aib-DYPQTDVGAGS-Hyp-NH₂ | | |
| 2-27 | U-ASHLSTACLGRLSACLH-(αMeK)-L- | 8, 15 | A1 |
| | Aib-DYPQTDVGAGS-(cis-P(4-NH₂))-NH₂ | | |
| 2-28 | U-ASHLSTACLGRLSACLH-(αMeK)-L- | 8, 15 | A1 |
| | Aib-DYPQTDVGAGS-(trans-P(4-NH₂))- | | |
| | NH₂ | | |
| 2-29 | U-ASHLSTACLGRLSACLH-(αMeK)-L- | 8, 15 | A1 |
| | Aib-DYPQTDVGAGS-p-NH₂ | | |
| 2-30 | U-ASHLSTACLG-homoR-LSACLH- | 8, 15 | A1 |
| | (αMeK)-L-Aib-DYPQTDVGAGS-Hyp- | | |
| | NH₂ | | |
| 2-31 | U-ASHLSTACLG-Orn-LSACLH-(αMeK)- | 8, 15 | A1 |
| | L-Aib-DYPQTDVGAGS-Hyp-NH₂ | | |
| 2-32 | U-ASHLSTA-HoC-LGRLSA-HoC-LH- | 8, 15 | A1 |
| | (αMeK)-L-Aib-DYPQTDVGAGS-Hyp- | | |
| | NH₂ | | |
| 2-33 | U-ASHLSTA-Pen-LGRLSA-Pen-LH- | 8, 15 | A1 |
| | (αMeK)-L-Aib-DYPQTDVGAGS-Hyp- | | |
| | NH₂ | | |
| 2-34 | U-ASHLSTA-(αMeC)-LGRLSA-(αMeC)- | 8, 15 | A1 |
| | LH-(αMeK)-L-Aib-DYPQTDVGAGS-Hyp- | | |
| | NH₂ | | |
| 2-36 | U-ASHLSTA-(N-Me-C)-LGRLSA-(N-Me- | 8, 15 | A1 |
| | C)-LH-(αMeK)-L-Aib-DYPQTDVGAGS- | | |
| | Hyp-NH₂ | | |
| 2-21 | U-ASHLSTAKLGRLSAKLH-(αMeK)-L- | 8, 15 | B1 |
| | Aib-DYPQTDVGAGS-Hyp-NH₂ | | |
| 2-22(AMY- | U-ASHLSTAVLGKLSAELHKL-Aib- | 11, 18 | B16 |
| 022, AMY- | DYPQTDVGAGS-Hyp-NH₂ | | |
| 040) | | | |
| 2-37 | U-ASHLSTA-Orn-LGRLSA-Orn-LH- | 8, 15 | B2 |
| | (αMeK)-L-Aib-DYPQTDVGAGS-Hyp- | | |
| | NH₂ | | |
| 2-38 | U-ASHLSTAkLGRLSAkLH-(αMeK)-L- | 8, 15 | B2 |
| | Aib-DYPQTDVGAGS-Hyp-NH₂ | | |
| 2-40 | U-ASHLSTA-Dab-LGRLSA-Dab-LH- | 8, 15 | B1 |
| | (αMeK)-L-Aib-DYPQTDVGAGS-Hyp- | | |
| | NH₂ | | |

According to an embodiment of the present disclosure, the polypeptide derivative has a structure as shown in any one of Table A or Table B:

TABLE A

| | | Binding sites | |
|---|---|---|---|
| Poly- | | (i, i + 7) of | |
| peptide | Amino acid sequence of | modifying | Modifying |
| name | polypeptide | groups | group |
| AMY-001 | Ac-ASHLSCAVLGRLCAELHKL-Aib- | 6, 13 | A1 |
| | DYPRTDVGAGSP-NH₂ | | |
| AMY-002 | Ac ASHLSTCVLGRLSCELHKL-Aib- | 7, 14 | A1 |
| | DYPRTDVGAGSP-NH₂ | | |
| AMY-003 | Ac-ASHLSTACLGRLSACLHKL-Aib- | 8, 15 | A1 |
| | DYPRTDVGAGSP-NH₂ | | |
| AMY-004 | Ac-ASHLSTAVCGRLSAECHKL-Aib- | 9, 16 | A1 |
| | DYPRTDVGAGSP-NH₂ | | |
| AMY-005 | Ac-ASHLSTAVLCRLSAELCKL-Aib- | 10, 17 | A1 |
| | DYPRTDVGAGSP-NH₂ | | |
| AMY-006 | Ac-ASHLSTAVLGCLSAELHCL-Aib- | 11, 18 | A1 |
| | DYPRTDVGAGSP-NH₂ | | |
| AMY-007 | Ac-ASHLSTAVLGRCSAELHKC-Aib- | 12, 19 | A1 |
| | DYPRTDVGAGSP-NH₂ | | |
| AMY-008 | Ac- | 13, 20 | A1 |
| | ASHLSTAVLGRLCAELHKLCDYPRTDVGA | | |
| | GSP-NH₂ | | |
| AMY-009 | Ac-ASHLSTACLGRLSACLH-Orn-L-Aib-D- | 8, 15 | A1 |
| | (αMeF)-PRTDVGAGS-Hyp-NH₂ | | |
| AMY-010 | Ac-ASH-(aMeK)-STACLGRLSACLHKL-Aib- | 8, 15 | A1 |
| | D-(αMeF)-P-(N-Me-R)-TDVGAGSP-NH₂ | | |
| AMY-011 | Ac-ASHLSTACLGRLSACLH-(αMeK)-L-Aib- | 8, 15 | A1 |
| | DYP-(N-Me-R)-TDVGAGS-Hyp-NH₂ | | |
| AMY-012 | Ac-ASHLSTACLG-homoR-LSACLH- | 8, 15 | A1 |
| | (αMeK)-L-Aib-DYP-(N-Me-R)- | | |
| | TDVGAGSP-NH₂ | | |
| AMY-013 | Ac-ASHLSTACLGRLSACLH-(αMeK)-L-Aib- | 8, 15 | A1 |
| | DYP-homoR-TDVGAGS-Hyp-NH₂ | | |
| AMY-014 | Ac-ASHLSTACLGRLSACLH-(αMeK)-L-Aib- | 8, 15 | A1 |
| | DYP-norR-TDVGAGS-Hyp-NH₂ | | |
| AMY-015 | Ac-ASHLSTACLGRLSACLH-(αMeK)-L-Aib- | 8, 15 | A1 |
| | DYPQTDVGAGS-Hyp-NH₂ | | |
| AMY-016 | Ac-ASHLSTACLGRLSACLH-(αMeK)-L-Aib- | 8, 15 | A12 |
| | DYPQTDVGAGS-Hyp-NH₂ | | |
| AMY-017 | Ac-ASHLSTACLGRLSACLH-(αMeK)-L-Aib- | 8, 15 | A5 |
| | DYPQTDVGAGS-Hyp-NH₂ | | |
| AMY-018 | Ac-ASHLSTACLGRLSACLH-(αMeK)-L-Aib- | 8, 15 | A4 |
| | DYPQTDVGAGS-Hyp-NH₂ | | |
| AMY-019 | Ac-ASHLSTACLGRLSACLH-(αMeK)-L-Aib- | 8, 15 | A1 |
| | DYP-r-TDVGAGS-Hyp-NH₂ | | |
| AMY-020 | Ac-ASHLSTAVLGCLSAELHCL-Aib- | 11, 18 | A1 |
| | DYPQTDVGAGS-Hyp-NH₂ | | |
| AMY-023 | Ac-KSHLSTACLGRLSACLH-(αMeK)-L-Aib- | 8, 15 | A1 |
| | DYP-(N-Me-R)-TDVGAGS-Hyp-NH₂ | | |
| AMY-024 | Ac-KSHLSTAVLGCLSAELHCL-Aib- | 11, 18 | A1 |
| | DYPQTDVGAGS-Hyp-NH₂ | | |
| AMY-025 | Ac-KSHLSTA-c-LGRLSA-c-LH-(αMeK)-L- | 8, 15 | A1 |
| | Aib-DYPQTDVGAGS-Hyp-NH₂ | | |
| AMY-026 | H-ASHLSTACLGRLSACLH-(αMeK)-L-Aib- | 8, 15 | A12 |
| | DYPQTDVGAGS-Hyp-NH₂ | | |
| AMY-027 | Ac-ASHLSTACLGRLSACLH-(αMeK)-L-Aib- | 8, 15 | A1 |
| | DYPQTDVGAGS-(cis-P(4-NH₂))-NH₂ | | |
| AMY-028 | Ac-ASHLSTACLGRLSACLH-(αMeK)-L-Aib- | 8, 15 | A1 |
| | DYPQTDVGAGS-(trans-P(4-NH₂))-NH₂ | | |
| AMY-029 | Ac-ASHLSTACLGRLSACLH-(αMeK)-L-Aib- | 8, 15 | A1 |
| | DYPQTDVGAGS-p-NH₂ | | |
| AMY-030 | Ac-ASHLSTACLG-homoR-LSACLH- | 8, 15 | A1 |
| | (αMeK)-L-Aib-DYPQTDVGAGS-Hyp-NH₂ | | |
| AMY-031 | Ac-ASHLSTACLG-Orn-LSACLH-(αMeK)-L- | 8, 15 | A1 |
| | Aib-DYPQTDVGAGS-Hyp-NH₂ | | |
| AMY-032 | Ac-ASHLSTA-HoC-LGRLSA-HoC-LH- | 8, 15 | A1 |
| | (αMeK)-L-Aib-DYPQTDVGAGS-Hyp-NH₂ | | |
| AMY-033 | Ac-ASHLSTA-Pen-LGRLSA-Pen-LH- | 8, 15 | A1 |
| | (αMeK)-L-Aib-DYPQTDVGAGS-Hyp-NH₂ | | |
| AMY-034 | Ac-ASHLSTA-(αMeC)-LGRLSA-(αMeC)- | 8, 15 | A1 |
| | LH-(αMeK)-L-Aib-DYPQTDVGAGS-Hyp- | | |
| | NH₂ | | |
| AMY-035 | Bzl-ASHLSTACLGRLSACLH-(αMeK)-L- | 8, 15 | A1 |
| | Aib-DYPQTDVGAGS-Hyp-NH₂ | | |
| AMY-036 | Ac-ASHLSTA-(N-Me-C)-LGRLSA-(N-Me- | 8, 15 | A1 |
| | C)-LH-(αMeK)-L-Aib-DYPQTDVGAGS- | | |
| | Hyp-NH₂ | | |

It should be noted that based on Table A, two amino acids connected by the modifying group in the polypeptides AMY-001 to AMY-020 and AMY-023 to AMY-036 are all replaced with K, and the modifying group is replaced with the modifying group represented by formula (V) (e.g., B1). The obtained polypeptide sequence has similar properties as AMY-001 to AMY-020 and AMY-023 to AMY-036 in Table A. Therefore, present disclosure does not show all of them, as they are all within the protection scope of present disclosure. The present disclosure only shows part of the polypeptides by way of example, see Table B for details. The specific activity data can be found in examples and test example sections in the present disclosure.

TABLE B

| | | Binding sites | |
|---|---|---|---|
| Poly- | | (i, i + 7) of | |
| peptide | Amino acid sequence of | modifying | Modifying |
| name | polypeptide | groups | group |
| AMY-021 | Ac-ASHLSTAKLGRLSAKLH-(αMeK)-L-Aib- | 8, 15 | B1 |
| | DYPQTDVGAGS-Hyp-NH₂ | | |
| AMY-022 | Ac-ASHLSTAVLGKLSAELHKL-Aib- | 11, 18 | B16 |
| | DYPQTDVGAGS-Hyp-NH₂ | | |
| AMY-037 | Ac-ASHLSTA-Orn-LGRLSA-Orn-LH-(αMeK)- | 8, 15 | B2 |
| | L-Aib-DYPQTDVGAGS-Hyp-NH₂ | | |
| AMY-038 | Ac-ASHLSTAkLGRLSAkLH-(αMeK)-L-Aib- | 8, 15 | B2 |
| | DYPQTDVGAGS-Hyp-NH₂ | | |
| AMY-039 | Ac-ASHLSTA-Dab-LGRLSA-Dab-LH-(αMeK)- | 8, 15 | B1 |
| | L-Aib-DYPQTDVGAGS-Hyp-NH₂ | | |
| AMY-040 | H-ASHLSTAVLGKLSAELHKL-Aib- | 11, 18 | B16 |
| | DYPQTDVGAGS-Hyp-NH₂ | | |
| AMY-041 | Bzl-ASHLSTAVLGKLSAELHKL-Aib- | 11, 18 | B16 |
| | DYPQTDVGAGS-Hyp-NH₂ | | |

It should be noted that the “Binding sites of the modifying groups” in Table A and Table B specifically refers the position i in Xᵢ in the structure represented by formula (I) of the present disclosure.

### Pharmaceutical Composition, Combination of Medicament or Kit

In a second aspect of the present disclosure, the present disclosure provides a pharmaceutical composition. According to an embodiment of the present disclosure, the pharmaceutical composition includes the polypeptide, derivative thereof, or the pharmaceutically acceptable salt thereof of the first aspect. As previously known, the polypeptide or the derivative thereof or the pharmaceutically acceptable salt thereof (referred to as a polypeptide or an analog thereof) is a dual agonist polypeptide analog, which can have exhibit balanced or unbalanced activating activities on both the amylin receptor (AMYR) and the calcitonin receptor (CTR) simultaneously. Therefore, a medicament containing the polypeptide, the derivative thereof, or the pharmaceutically acceptable salt thereof can effectively prevent or treat amylin receptor- and/or calcitonin receptor-related diseases.

According to an embodiment of the present disclosure, the pharmaceutical composition further includes pharmaceutically acceptable excipients or carriers.

In a third aspect of the present disclosure, the present disclosure provides a combination medicament or kit. According to an embodiment of the present disclosure, the combination medicament or kit includes: the polypeptide, the derivative thereof, or the pharmaceutically acceptable salt thereof of the first aspect, or the pharmaceutical composition in the second aspect as a first active ingredient; and a second active ingredient, wherein the second active ingredient includes a medicament for preventing and/or treating a disease. The combination medicament or kit according to the embodiment of the present disclosure can further improve a therapeutic effect on amylin receptor- and/or calcitonin receptor-related diseases.

According to an embodiment of the present disclosure, the diseases include amylin receptor- and/or calcitonin receptor-related diseases.

According to an embodiment of the present disclosure, the amylin receptor- and/or calcitonin receptor-related diseases include at least one of lipid metabolism disorders, glucose metabolism disorders, cardiovascular diseases, brain diseases, mental diseases, or neurological diseases.

According to an embodiment of the present disclosure, the amylin receptor- and/or calcitonin receptor-related diseases include at least one of metabolic disorder-related diseases, bone-related diseases, cardiovascular diseases unrelated to metabolic diseases, alcohol or medicament addiction-related symptoms or diseases, and neurodegenerative diseases.

According to an embodiment of the present disclosure, the amylin receptor- and/or calcitonin receptor-related diseases include at least one of diabetes mellitus, hypertension, arteriosclerosis, hepatic cirrhosis, coronary heart disease, angina, myocardial infarction, inflammatory bowel diseases, dyspepsia, gastrointestinal ulcers, hyperglycemia, impaired glucose tolerance, X syndrome, cognitive impairment, stroke, dyslipidemia-related diseases comprising hyperlipidemia and dyslipidemia, metabolic syndromes, non-alcoholic steatohepatitis, non-alcoholic fatty liver diseases, abnormal body weight, obesity, fatty liver disease, diabetic nephrotic syndromes, diabetes mellitus-related renal fibrosis, hepatic fibrosis, Alzheimer's disease, or Parkinson's disease.

According to an embodiment of the present disclosure, the amylin receptor- and/or calcitonin receptor-related diseases include at least one of cardiovascular diseases, diabetes mellitus and/or obesity.

According to an embodiment of the present disclosure, the medicament is selected from at least one of cardiovascular diseases, diabetes mellitus and/or obesity.

According to an embodiment of the present disclosure, the medicament is selected from at least one of adrenergic receptor blockers, HMG-CoA reductase inhibitors, angiotensin receptor antagonists, angiotensin-converting enzyme inhibitors, calcium channel blockers, endothelin antagonists, renin inhibitors, diuretics, aldosterone receptor blockers, endothelin receptor blockers, aldosterone synthase inhibitors, CETP inhibitors, relaxin, PCSK9 inhibitors, BNP and NEP inhibitors, GLP-1 analogs, insulin, sulfonylureas, biguanides, meglitazones, glucosidase inhibitors, DPP IV inhibitors, and SGLT2 inhibitors.

According to an embodiment of the present disclosure, the GLP-1 analogs include at least one of Semaglutide, Exenatide, Liraglutide, Dulaglutide, Tirzepatide, and Retatrutdie.

According to an embodiment of the present disclosure, the gliflozins (SGLT2 inhibitors) include at least one of dapagliflozin and empagliflozin.

According to an embodiment of the present disclosure, the biguanides include metformin.

### Uses and Method

In a fourth aspect of the present disclosure, the present disclosure provides a use of the polypeptide, the derivative thereof, or the pharmaceutically acceptable salt thereof according to the first aspect, or the pharmaceutical composition according to the second aspect, or the combination of medicament or kit according to the second aspect in the treatment or prevention of amylin receptor- and/or calcitonin receptor-related diseases; and/or in the manufacture of a medicament for treating or preventing amylin receptor- and/or calcitonin receptor-related diseases.

According to an embodiment of the present disclosure, the amylin receptor- and/or calcitonin receptor-related diseases include at least one of lipid metabolism disorders, glucose metabolism disorders, cardiovascular diseases, brain diseases, mental diseases, or neurological diseases.

According to an embodiment of the present disclosure, the amylin receptor- and/or calcitonin receptor-related diseases include at least one of metabolic disorder-related diseases, bone-related diseases, cardiovascular diseases unrelated to metabolic diseases, alcohol or drug addiction-related symptoms or diseases, and neurodegenerative diseases.

According to an embodiment of the present disclosure, the amylin receptor- and/or calcitonin receptor-related diseases include at least one of diabetes mellitus, hypertension, arteriosclerosis, hepatic cirrhosis, coronary heart disease, angina, myocardial infarction, inflammatory bowel diseases, dyspepsia, gastrointestinal ulcers, hyperglycemia, impaired glucose tolerance, X syndrome, cognitive impairment, stroke, dyslipidemia-related diseases comprising hyperlipidemia and dyslipidemia, metabolic syndromes, non-alcoholic steatohepatitis, non-alcoholic fatty liver diseases, abnormal body weight, obesity, fatty liver disease, diabetic nephrotic syndromes, diabetes mellitus-related renal fibrosis, hepatic fibrosis, Alzheimer's disease, or Parkinson's disease.

In a fifth aspect of the present disclosure, the present disclosure provides a method for treating or preventing amylin receptor- and/or calcitonin receptor-related diseases. According to an embodiment of the present disclosure, the method includes: administrating to a subject a pharmaceutically acceptable amount of the polypeptide, the derivative thereof, or the pharmaceutically acceptable salt thereof according to the first aspect, the pharmaceutical composition according to the second aspect or the combination of medicament or kit according to the second aspect. As previously known, the polypeptide, the derivative thereof, or the pharmaceutically acceptable salt thereof (referred to as a polypeptide or an analog thereof) is a dual agonist polypeptide analog, which can simultaneously exhibit certain balanced or unbalanced activating activities on both the amylin receptor (AMYR) and the calcitonin receptor (CTR). Therefore, the method of the present disclosure can effectively treat or prevent amylin receptor- and/or calcitonin receptor-related diseases.

According to an embodiment of the present disclosure, the amylin receptor- and/or calcitonin receptor-related diseases include at least one of metabolic disorder-related diseases, bone-related diseases, cardiovascular diseases unrelated to metabolic diseases, alcohol or medicament addiction-related symptoms or diseases, and neurodegenerative diseases.

According to an embodiment of the present disclosure, the metabolic disorder-related diseases include at least one of type 2 diabetes mellitus, dyslipidemia-related diseases, metabolic syndromes, non-alcoholic steatohepatitis, non-alcoholic fatty liver diseases, obesity, and fatty liver diseases.

According to an embodiment of the present disclosure, the neurodegenerative diseases include at least one of Alzheimer's disease and Parkinson's disease.

The effective amount of the polypeptide, the derivative thereof, the pharmaceutically acceptable salt thereof, or the pharmaceutical composition described in the present disclosure may vary with the mode of administration and the severity of a disease to be treated. The selection of the preferred effective amount can be determined by those of ordinary skill in the art according to various factors (e.g., through clinical tests). The factors include, but are not limited to the pharmacokinetic parameters of the active ingredient such as bioavailability, metabolism, and half-life; and the severity of the disease to be treated, the weight of the patient, the immune status of the patient, the route of administration, etc. For example, depending on the exigencies of treatment conditions, several divided doses may be given per day, or the dose may be reduced proportionally.

The polypeptide, the derivative thereof, the pharmaceutically acceptable salt thereof, or the pharmaceutical composition in the present disclosure may be incorporated into a medicament suitable for parenteral administration (e.g., intravenous, subcutaneous, intraperitoneal or intramuscular administration). These medicaments may be prepared in a variety of forms. Examples include liquid, semi-solid and solid dosage forms, including but not limited to: liquid solutions (e.g., injection solutions and infusion solutions) or lyophilized powders. Typical medicaments are in the form of injection solutions or infusion solutions. The polypeptide, the derivative thereof, the pharmaceutically acceptable salt thereof, or the pharmaceutical composition in the present disclosure may be administered by intravenous infusion, injection, intramuscular or subcutaneous injection.

The solutions of the present disclosure will be interpreted below in conjunction with the examples. Those skilled in the art will recognize that the following examples are only used to illustrate the present disclosure, but not to limit the scope of the present disclosure. Where specific techniques or conditions are not described in the examples, they are performed according to techniques or conditions described in the literature in the art or according to the product description. The reagents or instruments used without specifying manufacturers are all conventional products which are commercially available.

### Example 1: Preparation Method of Polypeptides or Derivatives Thereof

1. The linear peptide synthesis for polypeptides 1 to 20 (referred to as AMY-001 to AMY-020) and polypeptides 23 to 36 (referred to as AMY-023 to AMY-031) in Table A was performed via a classical Fmoc-tBu solid-phase synthesis method using a Symphony® X polypeptide synthesizer, and the reaction conditions were as follows.

(1) Resin swelling: Rink Amide MBHA resin was added to dichloromethane (DCM), and subjected to an N₂ sparging reaction at room temperature for 1 hour, followed by suction filtration; the resin was washed with DMF 2 to 3 times.

(2) Removal of Fmoc protecting group: a DMF (N,N-dimethylformamide) solution containing 20% (volume percentage, v/v) piperidine was added to the above resin, and subjected to an N₂ sparging reaction at room temperature for 10 minutes, followed by suction filtration; the above operation was repeated 2 to 3 times until complete deprotection was achieved; the resin was washed with DMF 2 to 3 times.

(3) Amino acid coupling: materials were fed according to a reaction ratio of resin:amino acids:DIC:Oxyma Pure=1:5:5:5 (equivalent ratio). The reactants, DIC (N,N′-diisopropylcarbodiimide) and Oxyma pure (condensation reagent) were dissolved in DMF in advance, subjected to an N₂ sparging reaction at room temperature for 10 minutes, then transferred to a resin reaction tank, and subjected to an N₂ sparging reaction at room temperature for 1 to 3 hours, followed by suction filtration after the reaction was completed; and the resin was washed with DMF 2 to 3 times. After the synthesis, the resin was washed with DCM 2 to 3 times and dried under vacuum to obtain peptide resin.

The above method was suitable for the following amino acids (D or L type) or the synthetic reagents, including but not limited to: Fmoc-AEEA-OH, Fmoc-Aib-OH, Fmoc-Ala-OH, Fmoc-Arg(Pbf)-OH, Fmoc-Asp(tBu)-OH, Fmoc-Cys(Trt)-OH, Fmoc-Gly-OH, Fmoc-Glu(tBu)-OH, Fmoc-Gln(Trt)-OH, Fmoc-His(Trt)-OH, Fmoc-Ile-OH, Fmoc-Leu-OH, Fmoc-Lys(Boc)-OH, Fmoc-Lys(Mtt)-OH, Fmoc-Lys(Alloc)-OH, Fmoc-Orn(Boc)-OH, Fmoc-Pro-OH, Fmoc-Phe-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-a-methyl-Leu-OH, Fmoc-a-methyl-Lys(Boc)-OH, Fmoc-α-methyl-Phe-OH, Fmoc-N-Me-Arg(Pbf)-OH, Fmoc-homoArg(Boc)-OH, Fmoc-cys(Trt)-OH, Fmoc-arg(Pbf)-OH, Fmoc-Pro(4-S—NH-Boc)-OH, Fmoc-Pro(4-R—NH-Boc)-OH, and Fmoc-pro-OH, etc.

2. Cleavage and drying of linear peptide: a polypeptide cleavage solution was prepared according to a volume ratio of trifluoroacetic acid (TFA):triisopropylsilylethynyl (TIPS):H₂O:EDT (1,2-ethanedithiol)=95:2:2:1. The cleavage solution (10 mL/g resin) was added to the dried peptide resin, shaken thoroughly on a shaker for 3 hours, and filtered to remove resin residues; 10 times the volume of cold MTBE (methyl tertiary butyl ether) was added to the filtrate; the resulting suspension was cooled at −20° C. for 1 hour, then centrifuged at 3500 rpm, washed and precipitated with cold MTBE 3 to 5 times, and dried under vacuum to obtain a crude peptide.

3. Purification of linear peptide: a solution was prepared according to a volume ratio of a mobile phase A (0.1% (volume percentage, v/v) TFA (trifluoroacetic acid)-water): a mobile phase B (0.1% (volume percentage, v/v) TFA-acetonitrile)=1.5:1; the crude peptide was dissolved in the solution to prepare a stock solution, which was then filtered with a 0.45 μm filter membrane. Gradient elution was performed using a C18 reversed-phase preparative column (20*250 mm, a particle size of 5 μm) with the mobile phase A and the mobile phase B at a flow rate of 10 mL/min. The target peaks were collected and a target peptide compound (i.e., linear peptide) was obtained after lyophilization.

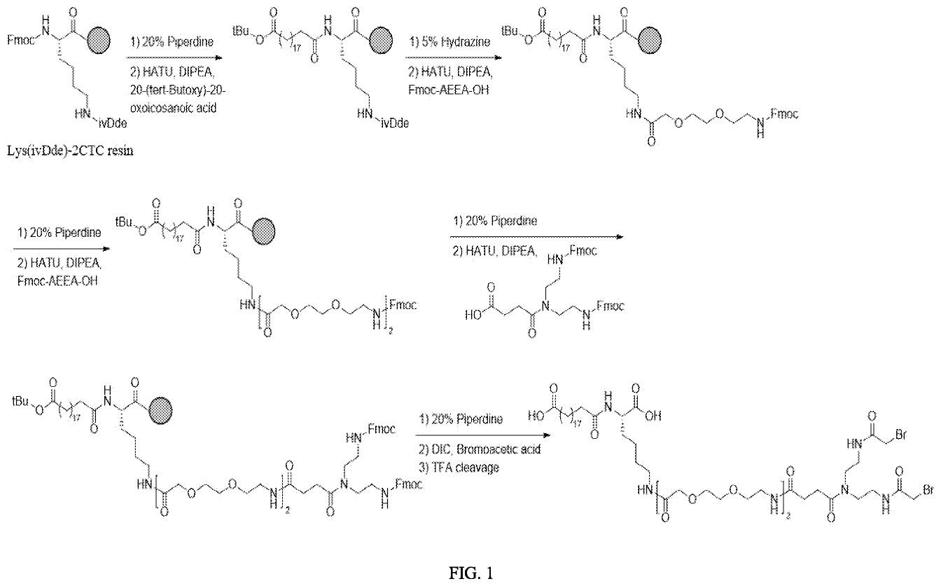

4. Preparation of Staple linker (or modifying group A): the Staple linker was prepared by a classical Fmoc-tBu solid-phase synthesis method. In this step, Staple-A1 (or modifying group A1) was taken as an example, and the reaction conditions were as follows (see FIG. 1 for the specific synthesis process).

(1) Resin swelling: Fmoc-L-Lys(ivDde)-2CTC resin was added to DCM, and subjected to an N₂ sparging reaction at room temperature for 1 hour, followed by suction filtration; the resin was washed with DMF 2 to 3 times.

(2) Removal of Fmoc protecting group: a DMF solution containing 20% piperidine was added to the above resin, and subjected to an N₂ sparging reaction at room temperature for 10 minutes, followed by suction filtration; the above operation was repeated until complete deprotection was achieved; the resin was washed with DMF 2 to 3 times.

(3) Fatty acid chain coupling: materials were fed according to a reaction ratio of resin:fatty acid:HATU(2-(7-azabenzotriazole)-N,N,N′,N′-tetramethylurea hexafluorophosphate):DIPEA(N,N-diisopropylethylamine)=1:5:5:10 (volume ratio). Fatty acid mono-tert-butyl ester, HATU and DIPEA were dissolved in DMF in advance, subjected to an N₂ sparging reaction at room temperature for 10 minutes, then transferred to a resin reaction tank, and subjected to an N₂ sparging reaction at room temperature for 1 to 3 hours, followed by suction filtration after the reaction was completed; the resin was washed with DMF 2 to 3 times.

(2) Removal of ivDde protecting group: a DMF solution containing 5% of hydrazine was added to the above resin, and subjected to an N₂ sparging reaction at room temperature for 10 minutes; the above operation was repeated 2 to 3 times until complete deprotection was achieved; the resin was washed with DMF 2 to 3 times.

(5) Coupling of amino acid, AEEA and bromoacetic acid: materials were fed according to a reaction equivalent ratio of resin:Fmoc-AA-OH:DIC:Oxyma Pure=1:5:5:5. The reactants, DIC and Oxyma pure were dissolved in DMF in advance, subjected to an N₂ sparging reaction at room temperature for 10 minutes, then transferred to a resin reaction tank, and subjected to an N₂ sparging reaction at room temperature for 1 to 3 hours, followed by suction filtration after the reaction was completed; the resin was washed with DMF 2 to 3 times; and after the synthesis, the resin was washed with DCM 2 to 3 times and dried under vacuum to obtain linker resin.

The structure of the Staple linker can be the above modifying groups A1 to A22, for example, the Staple linker can be selected from the following structures:

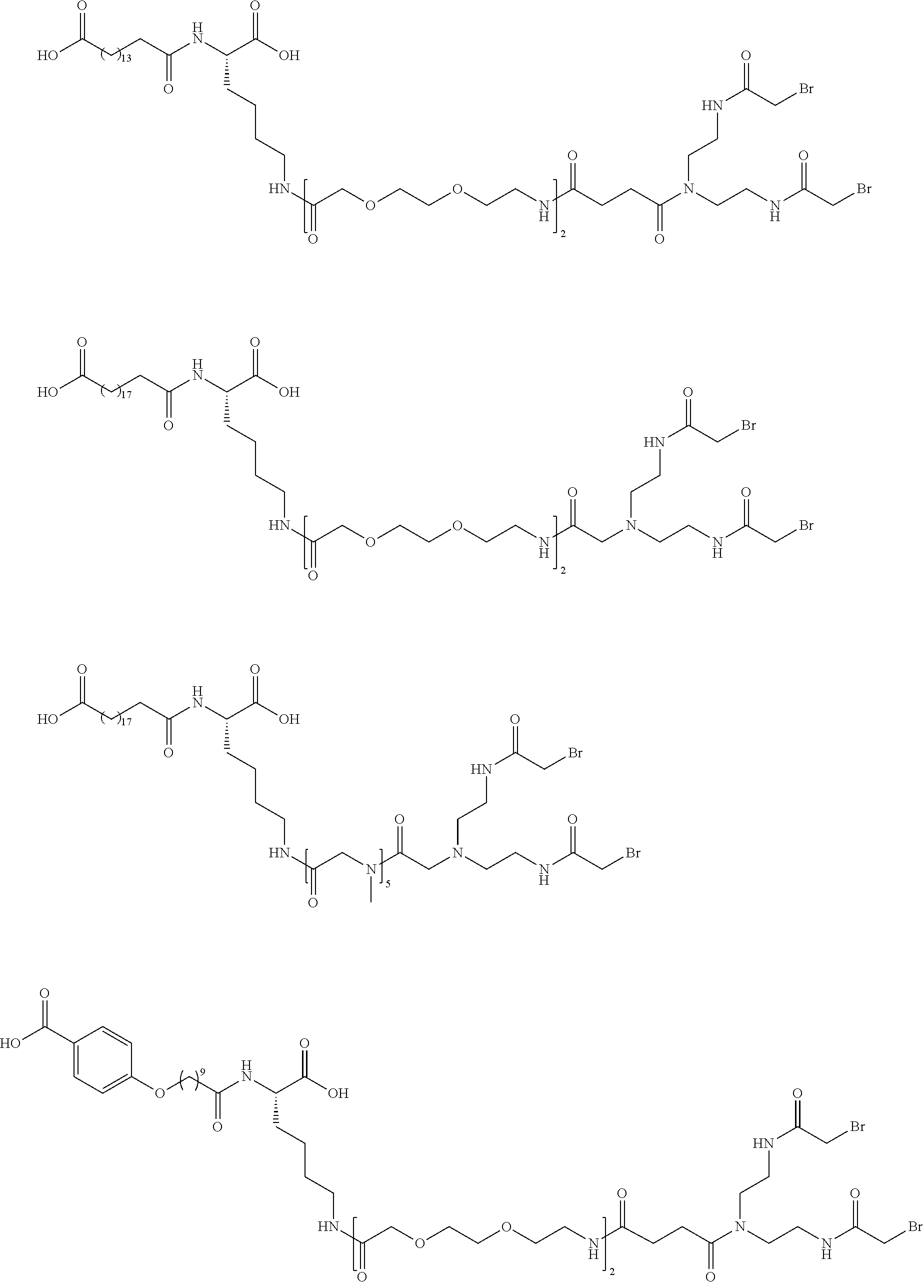

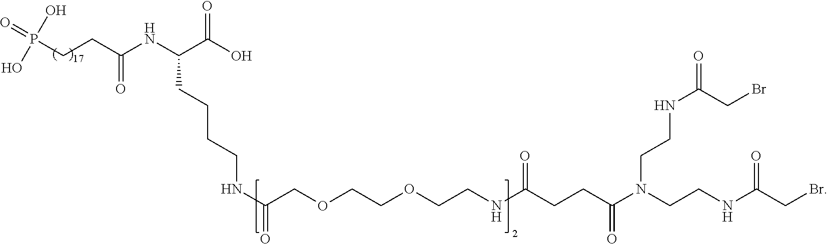

### 5. Coupling of Linear Peptide and Staple Linker:

1.0 equivalent of linear peptide (i.e., the target peptide compound obtained in step 3, in this step, a polypeptide DAC09 (AMY-008 for short) in Table A was taken as an example) and 1.2 equivalent of Staple linker peptide (i.e., the Staple linker obtained in step 4) were dissolved in a reaction solution (a final concentration of 1.2 mM) of PBS (phosphate buffer solution):acetonitrile at a volume ratio of 1:1.5; the pH of the reaction solution was adjusted to 8.0 using a 10% (volume percentage, v/v) NaOH aqueous solution, and the reaction solution was shaken thoroughly at room temperature on a shaker for 3 to 8 hours until the linear peptide was completely consumed as detected by LC-MS. The reaction solution was neutralized to pH 6.5 using a 5% (volume percentage, v/v) TFA aqueous solution, and filtered using a 0.45 μm filter membrane. Gradient elution was performed using a C18 reversed-phase preparation column (20*250 mm, a particle size of 5 μm) with the mobile phase A and the mobile phase B at a flow rate of 10 mL/min. The target peaks were collected and target coupling compounds (i.e., polypeptide derivatives, AMY-001 to AMY-020 and AMY-023 to AMY-031) were obtained after lyophilization. The detailed synthesis process was specifically shown as follows.

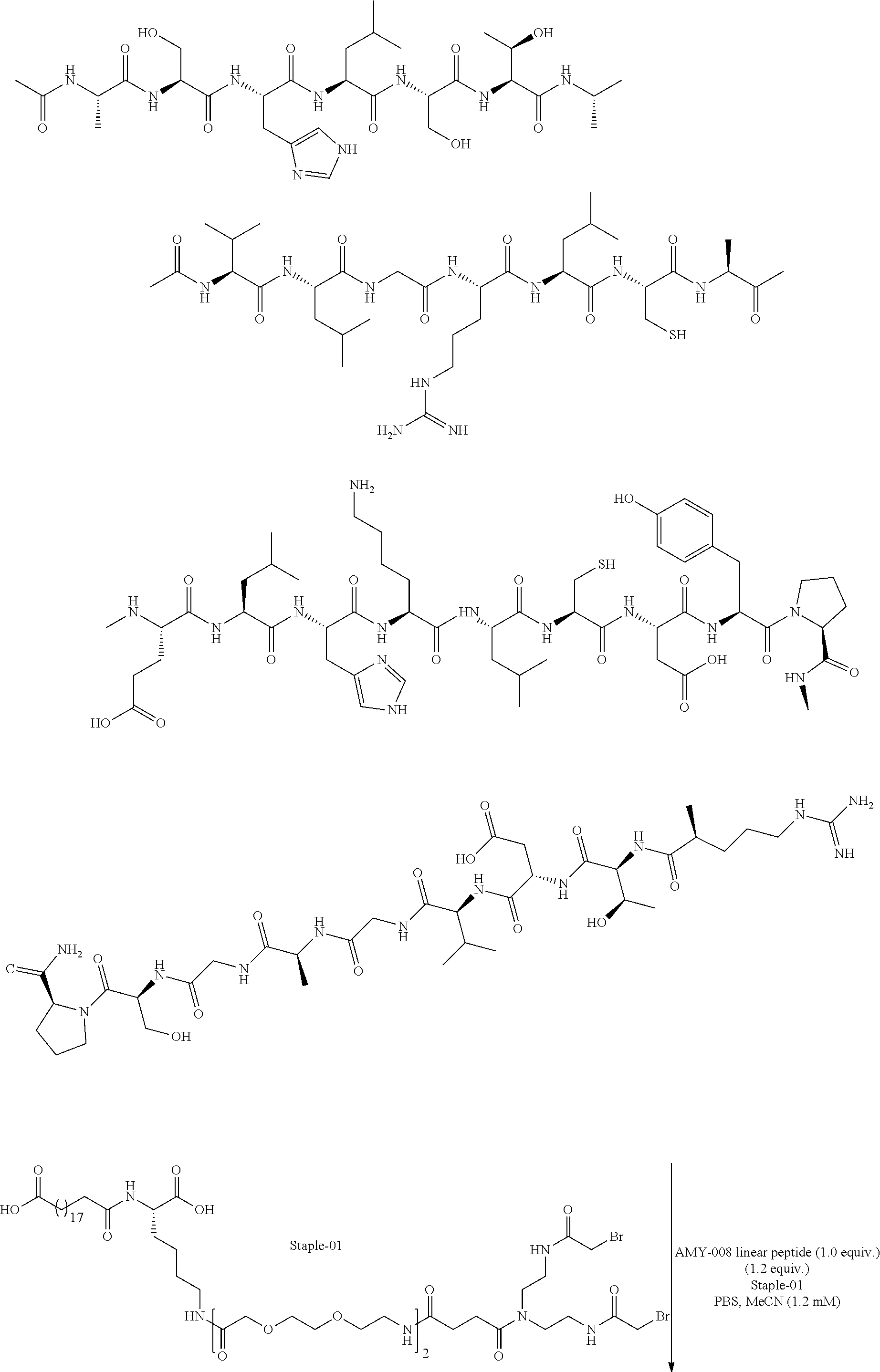

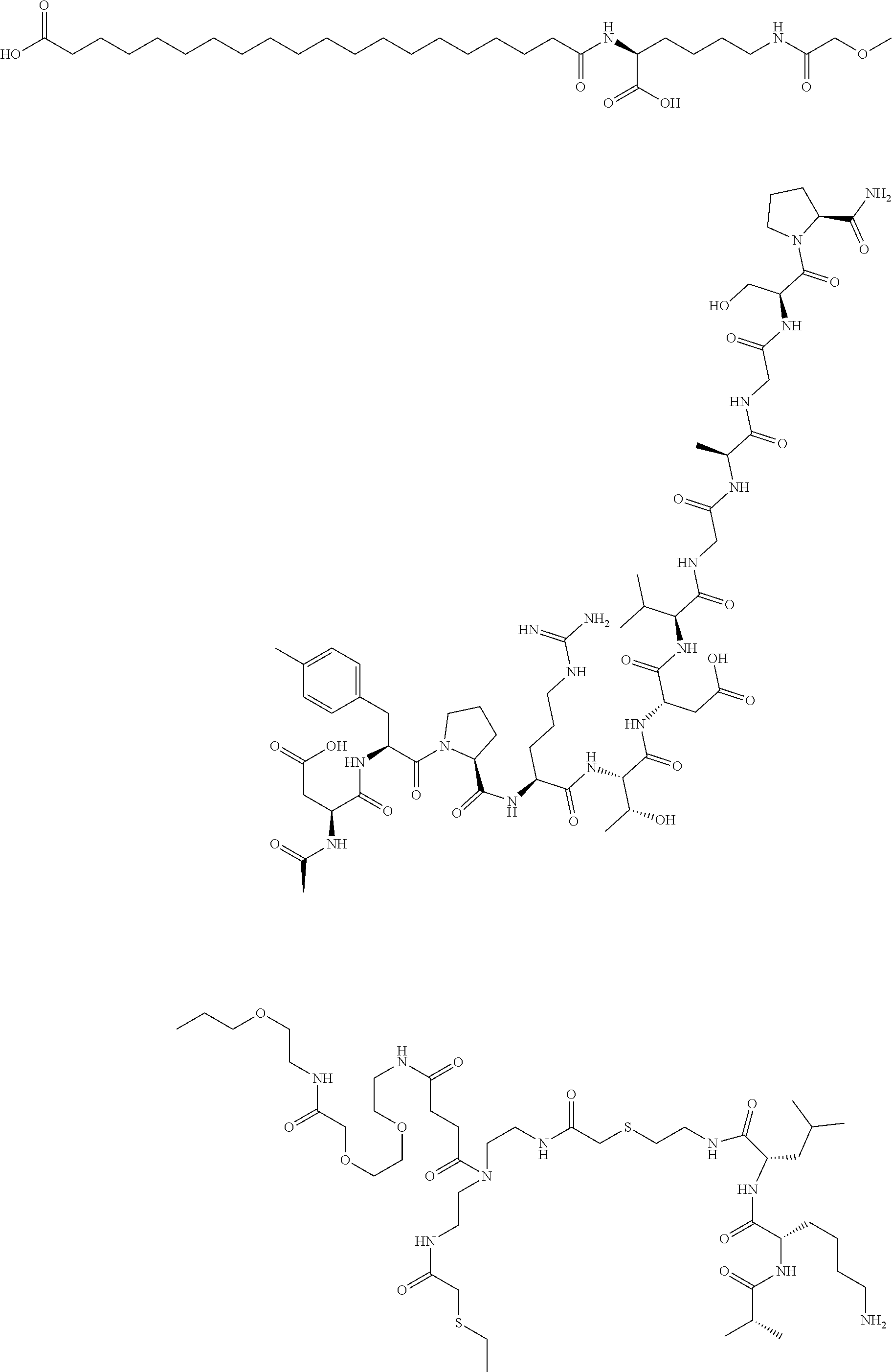

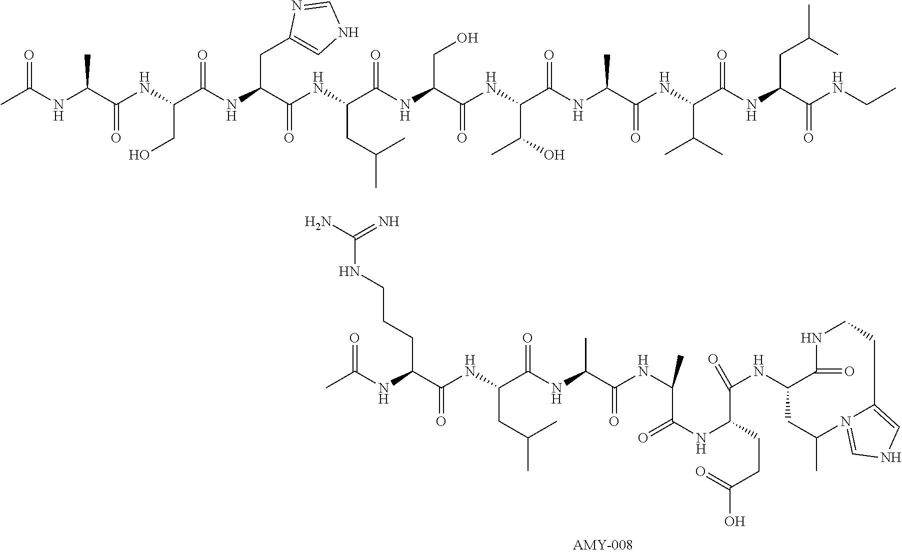

The purity and mass spectrometry identification were performed for each polypeptide derivative. As detected by HPLC, the purity of all polypeptide derivatives was greater than 90%; and the mass spectrometry identification confirmed that molecular weights of the polypeptides were all basically consistent with the theoretical molecular weights (all within the allowable error range). In this example, the molecular weights of some polypeptide molecules were exemplarily shown, and details can be found in Table 1.

TABLE 1

| | Calc MS | Found MS | Modifying |
|---|---|---|---|
| Polypeptide name | [M + 4H]⁴⁺ | [M + 4H]⁴⁺ | group |
| AMY-001 | 1098.52 | 1098.65 | A1 |
| AMY-002 | 1110.02 | 1110.44 | A1 |
| AMY-003 | 1088.50 | 1088.81 | A1 |
| AMY-004 | 1088.99 | 1089.28 | A1 |
| AMY-005 | 1097.02 | 1097.17 | A1 |
| AMY-006 | 1074.47 | 1074.52 | A1 |
| AMY-007 | 1088.99 | 1089.09 | A1 |
| AMY-008 | 1102.52 | 1102.83 | A1 |
| AMY-009 | 1088.75 | 1088.51 | A1 |
| AMY-010 | 1095.02 | 1095.19 | A1 |
| AMY-011 | 1099.52 | 1099.51 | A1 |
| AMY-012 | 1099.02 | 1098.94 | A1 |
| AMY-013 | 1099.52 | 1099.56 | A1 |
| AMY-014 | 1092.50 | 1092.37 | A1 |
| AMY-015 | 1089.00 | 1089.07 | A1 |
| AMY-016 | 1097.99 | 1097.90 | A12 |
| AMY-017 | 1080.48 | 1080.48 | A5 |
| AMY-018 | 1105.28 | 1105.15 | A4 |
| AMY-019 | 1096.01 | 1095.93 | A1 |
| AMY-020 | 1071.46 | 1071.33 | A1 |
| AMY-023 | 1163.84 | 1163.90 | A1 |

### Example 2: Preparation Method of Polypeptides or Derivatives Thereof

1. The linear peptide synthesis for polypeptides 21 to 22 (referred to as AMY-021 to AMY-022) and polypeptides 37 to 40 (referred to as AMY-037 to AMY-041) in Table A was performed by a classical Fmoc-tBu solid-phase synthesis method using a Symphony® X polypeptide synthesizer, and the reaction conditions were as follows.

(1) Resin swelling: Rink Amide MBHA resin was added to DCM, and subjected to an N₂ sparging reaction at room temperature for 1 hour, followed by suction filtration; the resin was washed with DMF 2 to 3 times.

(2) Removal of Fmoc protecting group: a DMF solution containing 20% (volume percentage, v/v) piperidine was added to the above resin, and subjected to an N₂ sparging reaction at room temperature for 10 minutes, followed by suction filtration; the above operation was repeated until complete deprotection was achieved; the resin was washed with DMF 2 to 3 times.

(3) Amino acid coupling: materials were fed according to a reaction ratio of resin:Fmoc-AA-OH:DIC:Oxyma Pure=1:5:5:5. Fmoc-AA-OH, DIC and Oxyma pure were dissolved in DMF in advance, subjected to an N₂ sparging reaction at room temperature for 10 minutes, then transferred to a resin reaction tank, and subjected to an N₂ sparging reaction at room temperature for 1 to 3 hours, followed by suction filtration after the reaction was completed; and the resin was washed with DMF 2-3 times. N-terminus amino acids in the sequence were Boc-L-His(Trt)-OH, Boc-L-Tyr(tBu)-OH and other amino acids, which were also synthesized by the above method. After the synthesis, the resin was washed with DCM 2 to 3 times and dried under vacuum to obtain peptide resin containing a linear peptide.

The above method was suitable for the following amino acids (D or L type) or synthetic reagents, including but not limited to: Fmoc-AEEA-OH, Fmoc-Aib-OH, Fmoc-Ala-OH, Fmoc-Arg(Pbf)-OH, Fmoc-Asp(tBu)-OH, Fmoc-Cys(Trt)-OH, Fmoc-Gly-OH, Fmoc-Glu(tBu)-OH, Fmoc-Gln(Trt)-OH, Fmoc-His(Trt)-OH, Fmoc-Ile-OH, Fmoc-Leu-OH, Fmoc-Lys(Boc)-OH, Fmoc-Lys(Mtt)-OH, Fmoc-Lys(Alloc)-OH, Fmoc-Orn(Boc)-OH, Fmoc-Pro-OH, Fmoc-Phe-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-a-methyl-Leu-OH, Fmoc-a-methyl-Lys(Boc)-OH, Fmoc-a-methyl-Phe-OH, Fmoc-N-Me-Arg(Pbf)-OH, Fmoc-homoArg(Boc)-OH, Fmoc-cys(Trt)-OH, Fmoc-arg(Pbf)-OH, Fmoc-Pro(4-S—NH-Boc)-OH, Fmoc-Pro(4-R—NH-Boc)-OH, Fmoc-pro-OH, etc.

2. Conjugation between a Staple linker (or referred to as modifying group B) and the peptide resin containing the linear peptide (AMY-022) obtained in step 1, the specific steps were as follows.

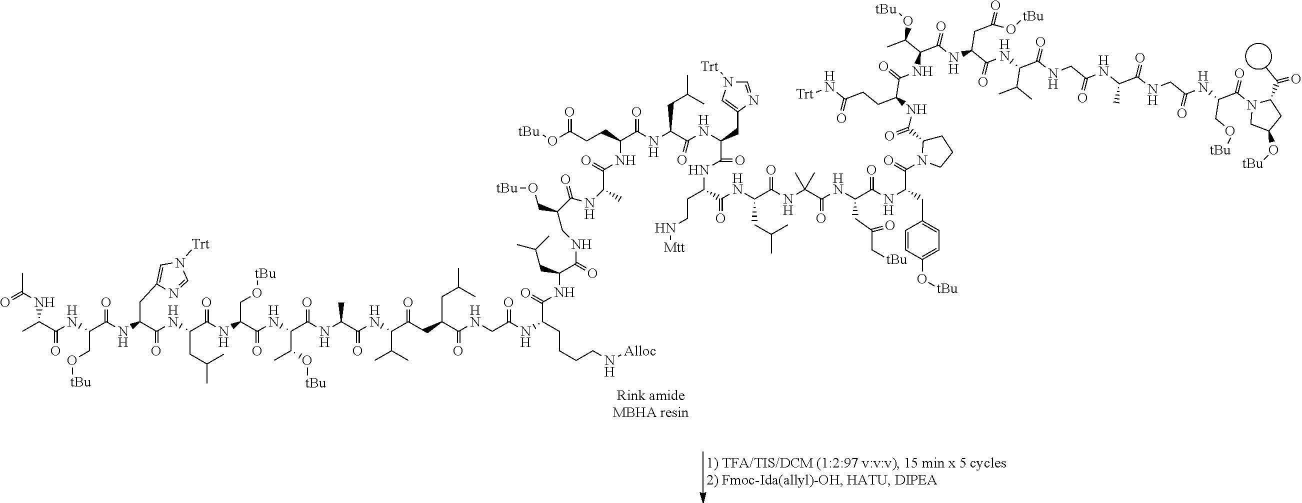

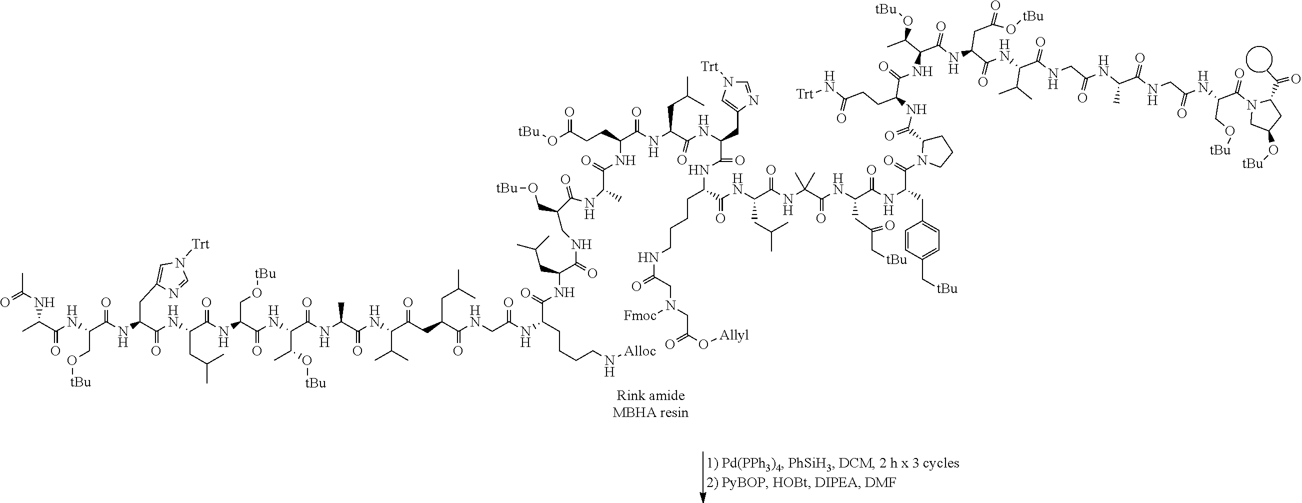

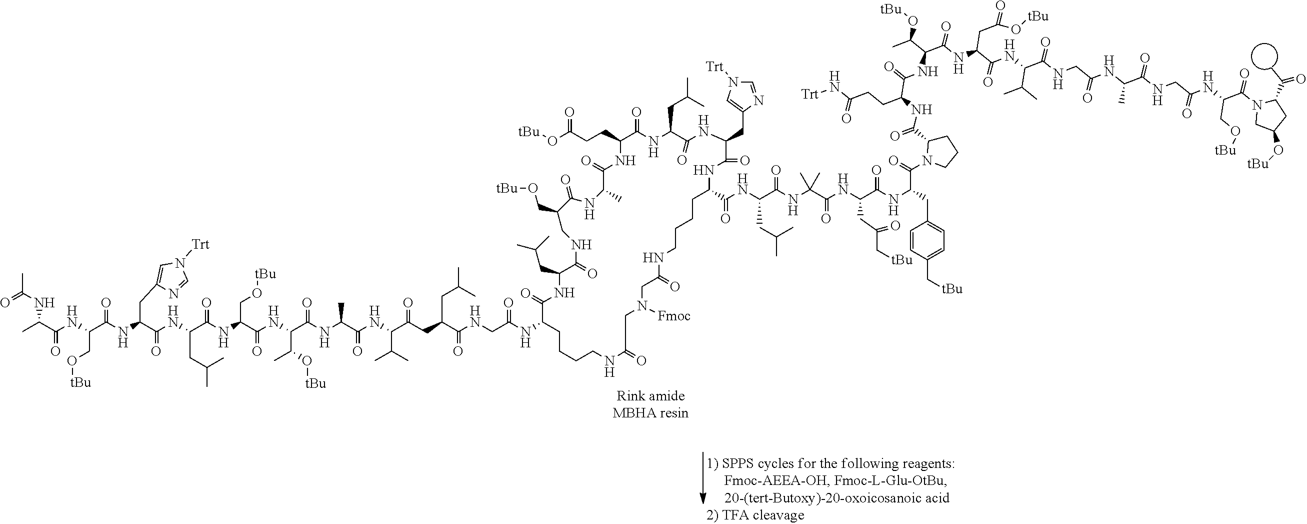

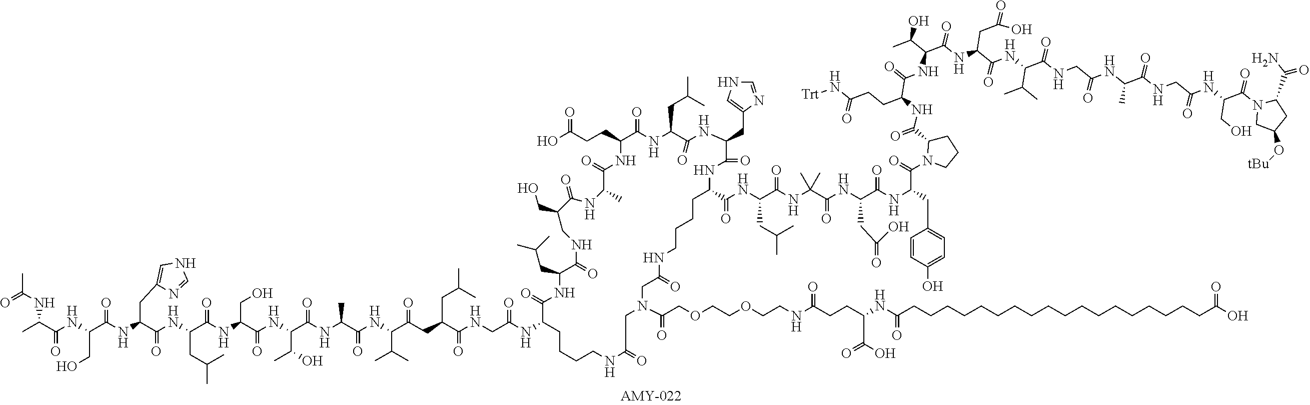

2.1 Removal of a Mtt protecting group from the peptide resin: a deprotection solution was prepared according to a volume ratio of TFA:TIPS:DCM=1:2:97; a Mtt removal solution (10 mL/g resin) was added to the resin, shaken thoroughly on a shaker for 15 minutes; the resin was subjected to suction filtration; this process was repeated 3 to 5 times until the removal solution no longer appears bright yellow after addition, indicating that complete removal; and the resin was washed with DMF 2 to 3 times.

2.2 Removal of Alloc and Allyl protecting groups from the peptide resin:DCM (10 mL/g resin), PhSiH3 (10 equivalents) and Pd(PPh3)4 (0.1 equivalent) were added to the resin, and subjected to an N₂ sparging reaction for 1 hour; the resin was subjected to suction filtration; this process was repeated 1 to 2 times; the resin was washed with DMF 2 to 3 times;

2.3 Cyclization reaction on the peptide resin:DMF (10 mL/g resin), PyBOP (5 equivalents), HOBt (5 equivalents) and DIPEA (10 equivalents) were added to the Alloc and Allyl protecting groups deprotected resin, and subjected to an N₂ sparging reaction for 4 hour; the resin was subjected to suction filtration; this process was repeated 1 to 2 times; and the resin was washed with DMF 2 to 3 times; and

2.4 Introduction of solid-phase side chain: this process was performed according to a standard solid-phase synthesis method, wherein the side chain has the following structure:

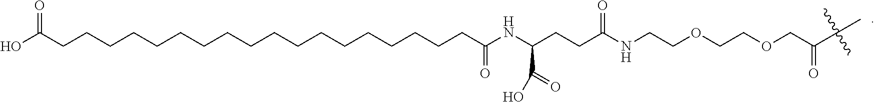 [SMILES: *C(=O)COCCOCCNC(=O)CC[C@H](NC(=O)CCCCCCCCCCCCCCCCCCC(=O)O)C(=O)O]

Furthermore, the structure of the Staple linker can be the above modifying groups B1 to B23.

3. Cleavage and drying: a polypeptide cleavage solution was prepared according to a volume ratio of TFA:TIPS:H₂O:EDT=95:2:2:1. The cleavage solution (10 mL/g resin) was added to the dried resin, shaken thoroughly on a shaker for 3 hours, and filtered to remove resin residues; 10 volumes of cold MTBE was added to the filtrate; the resulting suspension was cooled at −20° C. for 1 hour, then centrifuged at 3500 rpm, washed and precipitated with cold MTBE 3 to 5 times, and dried under vacuum to obtain a crude peptide.

4. Purification of polypeptide: a solution was prepared according to a volume ratio of a mobile phase A (0.1% TFA-water): a mobile phase B (0.1% TFA-acetonitrile)=1.5:1. The crude peptide was dissolved in the solution to prepare a stock solution, which was then filtered with a 0.45 μm filter membrane. Gradient elution was performed using a C18 reversed-phase preparation column (20*250 mm, a particle size of 5 μm) with the mobile phase A and the mobile phase B at a flow rate of 10 mL/min. Target peaks were collected and target peptide derivatives (i.e., AMY-021 to AMY-022) were obtained after lyophilization. The purity and mass spectrometry identification were performed for each polypeptide derivative. As detected by HPLC, the purity of all polypeptide derivatives was greater than 90%; and mass spectrometry identification confirmed that the molecular weights of the polypeptides were all basically consistent with the theoretical molecular weights (all within the allowable error range). In this example, the molecular weights of some polypeptide molecules were exemplarily shown, and details can be found in Table 2.

TABLE 2
Table of Compound Molecular weights

| | Calc MS | Found MS | Modifying |
|---|---|---|---|
| Polypeptide name | [M + 4H]⁴⁺ | [M + 4H]⁴⁺ | group |
| AMY-021 | 1076.5 | 1076.41 | B1 |
| AMY-022 | 1001.39 | 1001.46 | B16 |

### Test Example 1: In Vitro Activity Assay

1. The polypeptide derivatives prepared in Examples 1 and 2 of the present disclosure can bind to the target AMYR and CTR receptors on the cell membrane, activate the cAMP reaction element (CRE), and initiate the expression of luciferase downstream, where the expression level was positively correlated with the biological activity of the tested analogs. After activation, a luciferase substrate was added for chemiluminescence detection to measure the luminescence intensity, thereby characterizing the biological activity of the tested compounds. Therefore, the polypeptide derivatives prepared in Examples 1 and Example 2 were detected in the present disclosure, and the specific steps were as follows.

A stable transfected cell line of HEK293/pGM-CREB-L-Luc/AMY3R pool was constructed. The polypeptide derivatives (i.e., AMY-001 to AMY-041) prepared in Examples 1 and 2 were subjected to 5-fold serial dilution from a maximum of 1000 nM to prepare a dilution series containing 8 to 11 concentration gradients. Cagrilintide was selected as the positive control for testing, and a dilution series was prepared according to the same method for testing. The corresponding test cells were selected, digested (1 min), dispersed by pipetting and centrifuged (1000 rpm, 5 min). After discarding the medium, the cells were suspended with a freestyle medium, and centrifuged. The cells were collected and counted, diluted to a cell density of 5*10⁵/mL, and added to detection wells of a 384-well plate (40 μL/20000 cells/well). The dilution series of the corresponding analyte was added using Echo. The cell plate was incubated under the conditions of 37° C. and 5% carbon dioxide for 6 hours, then added with ONE-GLO (20 μL/well) for detection. The cell plate was placed in a dark place for 3 minutes, and measured with a chemiluminescence microplate reader. The plate was read within 30 minutes to record measurement results. An activation curve was plotted using GraphPad Prism software to calculate an EC₅₀ value of a compound. The results showed that the polypeptide derivatives of the present disclosure exhibited binding activities on both AMYR and CTR.

2. The polypeptide derivatives prepared in Example 1 and Example 2 of the present disclosure can bind to the target AMYR and CTR receptors on the cell membrane, activate the receptors and release cAMP. Therefore, the activity of the analyte can be determined by using an HTRF (homogeneous time-resolved fluorescence) method and using a cAMP detection kit. The specific steps were as follows:

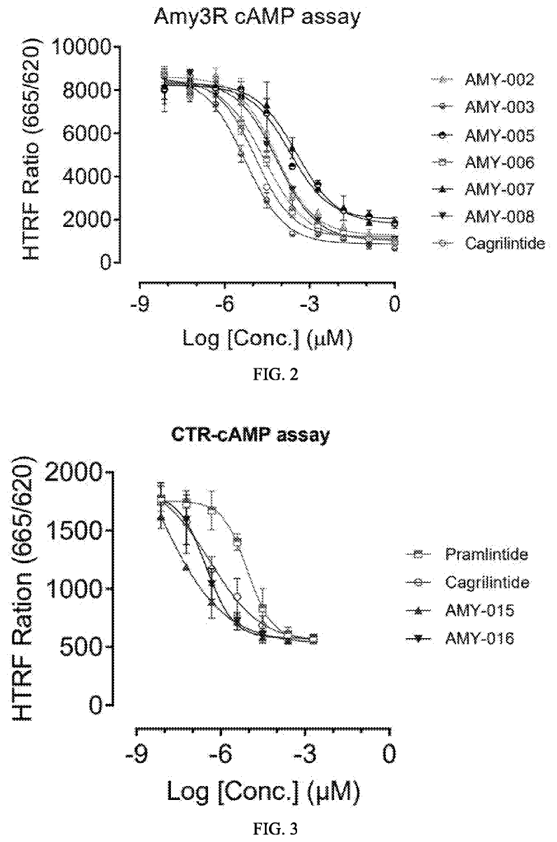

A stable transfected cell line of HEK293/pGM-CREB-L-Luc/AMY3R pool and HEK293/pGM-CREB-L-Luc/CTR pool was constructed. The polypeptide derivatives (i.e., AMY-001 to AMY-041) prepared in Examples 1 and 2 were subjected to 5-fold serial dilution from a maximum of 1000 nM to prepare a dilution series containing 8 to 11 concentration gradients. Cagrilintide and sCT were selected as positive controls for testing, and a dilution series was prepared according to the same method for testing. The test cell lines were selected and treated with cAMP-specific trypsin for digestion, and then suspended with 3 mL of serum-free DMEM medium by pipetting, and the cells were counted. 5 μL of IBMX (prepared with serum-free DMEM medium, 0.5 mM) and test cells (5 μL/7500 cells/well) were sequentially added to a 384-well plate, incubated under the conditions of 37° C., 5% CO₂ for 30 minutes, then added with cAMP-d2 (5 μL, 1×) and Anti-cAMP-Cryptate (5 μL, 1×) and placed at room temperature for 1 hour. A HTRF value was determined by a chemiluminescence microplate reader, and the results were recorded. An activation curve was plotted using GraphPad Prism software to calculate an IC₅₀ value of each polypeptide derivative. The results showed that the polypeptide derivatives of the present disclosure exhibited binding activities on both AMY3R and CTR. The IC₅₀ values of some polypeptides were exemplarily shown in this example, see Table 3, FIG. 2 and FIG. 3 for details.

TABLE 3
IC₅₀ values for target peptide derivatives

IC₅₀ (pM) of receptor

| Polypeptide name | AMY3R | CTR |
|---|---|---|
| Positive control Cagrilintide | 12.3 | 0.4 |
| Positive control Pramlintide | ND | 8.7 |
| AMY-002 | 67.1 | 90.6 |
| AMY-003 | 5.2 | 100.4 |
| AMY-005 | 198.4 | 302.6 |
| AMY-006 | 25.4 | 10.7 |
| AMY-007 | 349.1 | 262.1 |
| AMY-008 | 71.3 | 50.2 |
| AMY-009 | 41.4 | 89.4 |
| AMY-010 | 52.3 | 41.5 |
| AMY-011 | 4.8 | 12.2 |
| AMY-012 | 13.3 | 35.7 |
| AMY-015 | 0.9 | 0.1 |
| AMY-016 | 1.1 | 0.3 |
| AMY-021 | 4.4 | 1.2 |
| AMY-022 | 67.9 | 101.7 |

### Test Example 2: Pharmacokinetic Evaluation

1. The pharmacokinetic behaviors of the polypeptide derivatives (i.e., AMY-001 to AMY-041) prepared in Example 1 and Example 2 in SD rats were tested.

Each polypeptide derivative (i.e., AMY-001 to AMY-041) was administered to male SD rat as a single injection via subcutaneous injection (SC, 3 mg/kg, with PBS as a medicament vehicle, a concentration of 1.5 mg/mL, and an administration volume of 2 mL) and intravenous injection (IV, 1 mg/kg, with PBS as a medicament vehicle, a concentration of 0.5 mg/mL, and an administration volume of 2 mL), respectively. After administration, whole blood samples were respectively collected from the jugular vein or other appropriate veins at selected time points. 0.2 mL of blood was collected and placed in a labeled EDTA-K2 anticoagulant tube. The tube was gently inverted up and down to ensure thorough mixing of the anticoagulant (EDTA-K2) with the blood, immediately placed on wet ice and centrifuged as soon as possible to separate plasma, and the centrifugation conditions were set to 4° C., 6800 g, and 6 minutes. Plasma samples were stored in a refrigerator at a temperature not exceeding −20° C. until subsequent analysis.

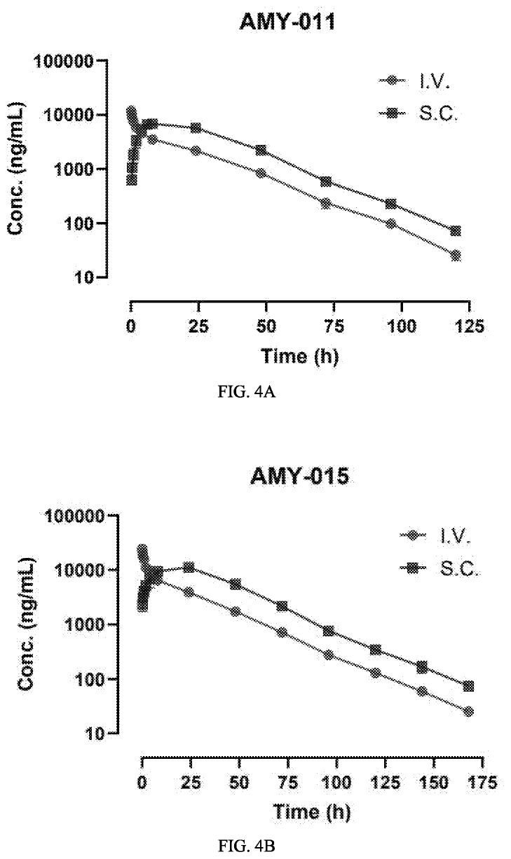

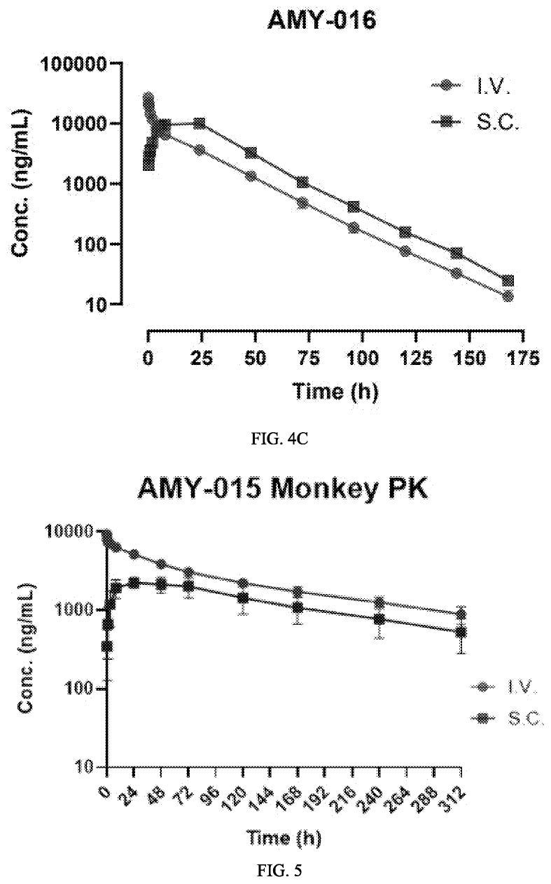

Under yellow light conditions in an ice water bath, (1) except for the blank sample, 300 μL of precipitant containing internal standard was added to a 96-well plate containing 20 μL of a standard curve sample, a quality control sample or an unknown sample; 300 μL of acetonitrile was added to the blank sample; (2) vortex mixing; (3) centrifugation; (4) 150 μL of supernatant was taken and added into a new 96-well plate, and then mixed well with 150 μL of ultrapure water; and (5) The above samples were quantitatively analyzed by using LC-MS analysis. Gradient elution was performed on a XB—C18 chromatographic column (2.1*100 mm, 3 μm), with the mobile phase as follows: mobile phase A=0.1% (volume percentage, v/v) formic acid-water; and mobile phase B=acetonitrile. Pharmacokinetic parameters were calculated using Phoenix WinNonlin software. The results showed that the polypeptide derivatives of the present disclosure exhibited good in vivo absorption, relatively high blood concentration and relatively long half-life in rats. The detection results of the pharmacokinetic parameters of some polypeptide derivatives were shown in Table 4 and FIG. 4.

TABLE 4
PK data of some polypeptide derivatives in rats

| | | T_{1/2} | Tₘₐₓ | Cₘₐₓ | AUC_{inf} | Cl | F |
|---|---|---|---|---|---|---|---|
| Compound | Dose | (h) | (h) | (ng/mL) | (h*ng/mL) | (mL/min/kg) | (%) |
| AMY-011 | IV (1 mpk) | 15.5 | — | — | 142969 | 0.117 | — |
| | SC (3 mpk) | 15.6 | 8 | 6830 | 282700 | — | 65.9 |
| AMY-015 | IV (1 mpk) | 20.5 | — | — | 285707 | 0.0587 | — |
| | SC (3 mpk) | 20.6 | 24 | 11147 | 566640 | — | 66.1 |
| AMY-016 | IV (1 mpk) | 17.4 | — | — | 260010 | 0.0641 | — |
| | SC (3 mpk) | 18.0 | 24 | 10077 | 452259 | — | 58.0 |

2. The pharmacokinetic behaviors of the polypeptide derivatives (i.e., AMY-001 to AMY-041) prepared in Example 1 and Example 2 in Cynomolgus macaques were tested.

Each polypeptide derivatives (i.e., AMY-001 to AMY-041) was respectively administered to adult male Cynomolgus macaques as a single injection via subcutaneous injection (S.C., 0.2 mg/kg, with PBS as a medicament vehicle, a concentration of 0.2 mg/mL, and an administration volume of 1 mL) and intravenous injection (I.V., 1 mg/kg, with PBS as a medicament vehicle, a concentration of 0.2 mg/mL, and an administration volume of 1 mL), respectively. After administration, whole blood samples were respectively collected from the jugular vein or other appropriate veins at selected time points. 0.2 mL of blood was collected and placed in a labeled EDTA-K2 anticoagulant tube. The tube was gently inverted up and down to ensure thorough mixing of the anticoagulant (EDTA-K2) with the blood, immediately placed on wet ice and centrifuged as soon as possible to separate plasma, and the centrifugation conditions were set to 4° C., 6800 g, and 6 minutes. Plasma samples were stored in a refrigerator at a temperature not exceeding −20° C. for subsequent analysis.

Under yellow light conditions in an ice water bath, (1) except for the blank sample, 300 μL of precipitant containing internal standard was added to a 96-well plate containing 20 μL of a standard curve sample, a quality control sample or an unknown sample; 300 μL of acetonitrile was added to the blank sample; (2) vortex mixing; (3) centrifugation; (4) 150 μL of supernatant was taken and added into a new 96-well plate, and then mixed well with 150 μL of ultrapure water; and (5) the above samples were quantitatively analyzed by using LC-MS analysis. Gradient elution was performed on a XB—C18 chromatographic column (2.1*100 mm, 3 μm), with the mobile phase as follows: mobile phase A=0.1% formic acid-water; and mobile phase B=acetonitrile. Pharmacokinetic parameters were calculated using Phoenix WinNonlin software. The results showed that the polypeptide derivatives of the present disclosure exhibited good in vivo absorption, relatively high blood concentration and relatively long half-life in rats. The detection results of the pharmacokinetic parameters of some polypeptide derivatives were shown in Table 5 and FIG. 5.

TABLE 5
PK data of some polypeptide derivatives in Cynomolgus macaques

| | | T_{1/2} | Tₘₐₓ | Cₘₐₓ | AUC_{inf} | Cl | F |
|---|---|---|---|---|---|---|---|
| Compound | Dose | (h) | (h) | (ng/mL) | (h*ng/mL) | (mL/min/kg) | (%) |
| AMY-015 | IV (0.2 mpk) | 146 | — | — | 942419 | 0.00361 | — |
| | SC (0.2 mpk) | 143 | 24 | 2257 | 513953 | — | 54.5 |

### Test Example 3: Changes in Body Weight and Food Intake after Single Administration in Mild SD Rats

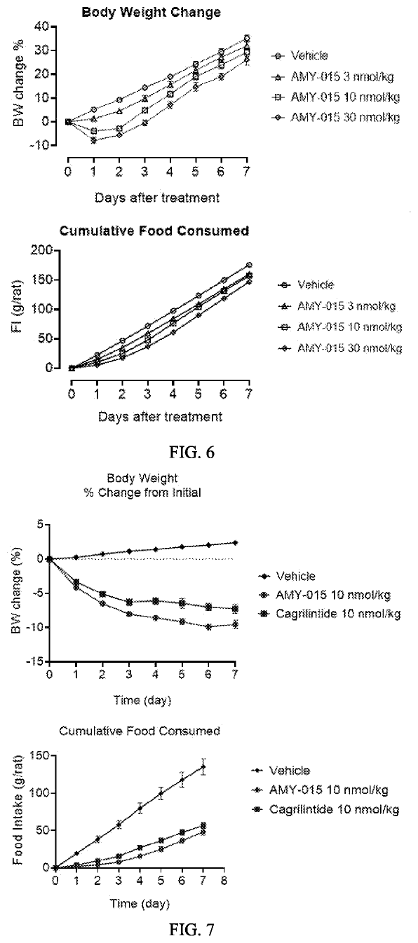

Adult male wild-type SD rats each weighing 200-250 g were selected for test. The SD rats were randomly divided into groups, 4 rats per group (2 rats in each cage) and acclimated for one week with normal diet and free access to water. After acclimation, a baseline blood glucose value and body weight value were measured for each rat before administration (Day 0). Subsequently, a vehicle control and three doses of polypeptide derivatives (AMY-001 to AMY-041) (3 nmol/kg, 10 nmol/kg, and 30 nmol/kg) prepared in Example 1 and Example 2 of the present disclosure were respectively administrated via subcutaneous injection (“s.c.”, synonymous with “S.C.”) according to experimental grouping. After administration, the rats were allowed free access to normal diet and water. On Days 1, 2, 3, 4, 5, 6, and 7 after administration, the body weights of each rat and the total daily intake of each group were detected, respectively. The body weight (% starting value) of each rat and a change value of cumulative food intake of each group were calculated, and curves were plotted. The results showed that the compound of the present disclosure can reduce the body weights and food intake of rats. In this test example, the results of AMY-015 were exemplarily displayed, as illustrated in FIG. 6.

### Test Example 4: Changes in Body Weight, Blood Glucose and Food Intake after Continuous Administration in DIO Rat Model

Adult male Sprague Dawley rats each weighing 200-250 g were selected for test. The rats were fed with a high-fat diet for 16 weeks and each weighed about 600-700 g. The above rats were randomly divided into groups (5 rats in each group) and acclimated in individual cages for one week with normal diet and free access to water. After acclimation, a baseline weight value of each rat was detected before administration (Day 0). Subsequently, a vehicle control, Cagrilintide (1 nmol/kg, 3 nmol/kg, and 10 nmol/kg, Q2D) and AMY-015 (1 nmol/kg, 3 nmol/kg, and 10 nmol/kg, Q2D) were respectively administrated via subcutaneous injection (s.c.). After administration, the rats were allowed free access to normal diet and water. On Days 1, 2, 3, 4, 5, 6, and 7 after administration, the body weights of each rat and the total daily food intake of each group were detected, respectively. The body weight (% starting value) of each rat and the change value of cumulative food intake of each group were calculated, and curves were plotted. The results showed that the compound of the present disclosure can reduce the body weights and food intake of rats. In this test example, the results of AMY-015 were exemplarily displayed, as illustrated in FIG. 7.

### Test Example 5: Changes in Body Weight and Adipose Tissues in DIO Rat Model after Continuous Administration Alone or in Combination with Semaglutide

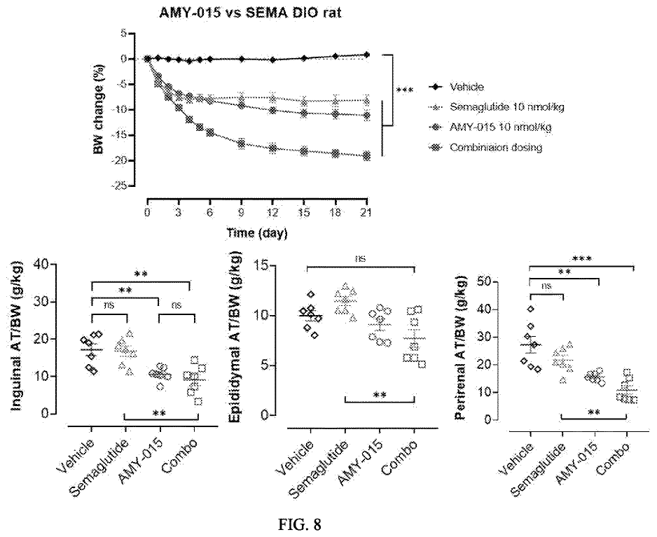

Adult male Sprague Dawley rats each weighing 200-250 g were selected for test. The SD rats were fed a high-fat diet for 20 weeks and each weighed about 600-700 g. The above rats were randomly divided into groups (7 rats in each group) and acclimated in individual cage for one week with normal diet and free access to water. After acclimation, the baseline blood glucose value and body weight value of each rat were detected before administration (Day 0). Subsequently, a vehicle control, Semaglutide (10 nmol/kg, QD), Cagrilintide (10 nmol/kg, Q2D), AMY-015 (10 nmol/kg, Q2D) and Semaglutide (10 nmol/kg, QD) combined with AMY-015 (10 nmol/kg, Q2D) were administrated via subcutaneous injection (s.c.) for consecutive 21 days. The body weight of each rat and the total daily food intake of the rats in each group were measured daily after administration, the body weight (% starting value) of each rat and the change value of total daily food intake of the rats in each group were calculated, and curves were plotted. After administration, blood and adipose tissues were collected by dissection, adipose tissues were weighed, and biochemical indicators in the blood were detected. The results showed that the compounds of the present disclosure had a superior weight loss effect compared to Cagrilintide. Moreover, when combined with Semaglutide, they are more effective in reducing the body weight and food intake of rats, while protecting muscles from loss to a certain extent. In this test example, the results of AMY-015 were exemplarily displayed, as illustrated in FIG. 8.

### Test Example 6: Changes in Body Weight, Blood Glucose and Food Intake after Continuous Administration in ZDF Rat Model

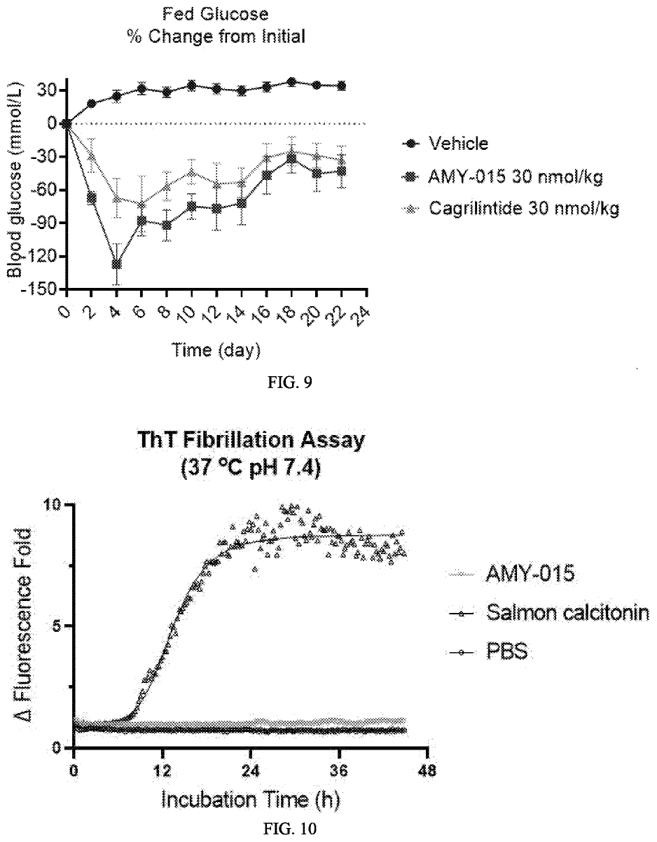

Adult male ZDF rats each weighing 350-400 g were selected for test. The rats were fed with a diabetes-inducing diet. The above rats were randomly divided into groups (8 rats in each group) and acclimated in individual cages for one week with normal diet and free access to water. After acclimation, a baseline blood glucose and body weight value of each rat were detected before administration (Day 0). Subsequently, a vehicle control, Cagrilintide (30 nmol/kg, Q2D) and AMY-015 (30 nmol/kg, Q2D) were respectively administrated via subcutaneous injection (s.c.) for consecutive 28 days. After administration, the blood glucose levels of each rat was measured once every two days, the change value of blood glucose (% starting value) of the rat was calculated, and a blood glucose-time curve was plotted. The results showed that the compounds of the present disclosure can reduce the blood glucose in rats, which was superior to Cagrilintide. In this test example, the results of AMY-015 were exemplarily displayed, as illustrated in FIG. 9.

### Test Example 7: Thioflavin T (ThT) Fibrillation Experiment

Amylin analogs are prone to cause fibrillation aggregation both in vivo and in vitro due to their structural characteristics, resulting in stringent storage conditions and potential immunogenic risks. In the present disclosure, a helix fixation technology was adopted to stabilize the secondary structure of a peptide chain, and reduce the inter-chain hydrophobic group stacking, effectively reducing the tendency of fibrillation. APBS solution (1 mg/mL) of a polypeptide to be tested and a PBS working solution of ThT (5 mM) were prepared. 400 μL of solution of each polypeptide sample to be tested (the polypeptide derivatives prepared in Example 1 and Example 2 (i.e., AMY-001 to AMY-041)) was taken into a 1.5 mL EP tube at room temperature, added with 8 μL of ThT stock solution, vortex-mixed well, and centrifuged at low speed to ensure no liquid adhered to the tube wall. The liquid was pipetted into a 96-well plate at 200 μL per well and multiple parallel groups could be set up. 200 μL of PBS was added to peripheral wells surrounding the samples for edge sealing, and the plate was covered with a transparent film. Kinetic test was performed using a Biotek microplate detector with a monitoring interval of 15 min, an excitation wave of 440 nm, an emission wave of 485 nm. ΔFold change was (detection value/average of the first ten detection points), the abscissa represented time (h), and the ordinate represented A Fold change. By using Graphpad nonlinear fitting to plot graphs, it was found that the polypeptide derivatives of the present disclosure can significantly reduce fibrillation aggregation and have higher stability. In this example, the results of some polypeptide derivatives were exemplarily shown, as illustrated in FIG. 10 for details.

In the description of the present specification, the description of referring terms such as “an embodiment”, “some embodiments”, “an example”, “a specific example” and “some examples” mean that particular features, structures, materials or characteristics described in combination of the embodiments or examples are included in at least one embodiment or example of the present disclosure. In the specification, the schematic descriptions of the above terms do not necessarily refer to the same embodiment or example. Furthermore, the described particular features, structures, materials or characteristics can be combined in any suitable manner in one or more embodiments or examples. In addition, where there is no mutual contradiction, a person skilled in the art can integrate and combine different embodiments or examples described in this specification as well as the features of different embodiments or examples described in this specification.

Although the examples of the present disclosure have been shown and described above, it can be understood that the above examples are exemplary and should not be construed as limiting the present disclosure. A person of ordinary skill in the art can make changes, modifications, substitutions and variations to the above-mentioned examples within the scope of the present disclosure.

What is claimed is:

1. A polypeptide, derivative thereof, or pharmaceutically acceptable salt thereof, having a structure represented by formula (I):
U—X₁SHX₄SX₆X₇X₈X₉X₁₀X₁₁X₁₂X₁₃X₁₄X₁₅X₁₆X₁₇X₁₈X₁₉X₂₀DX₂₂PX₂₄TDVGAGSX₃₂—NH₂ (I), wherein:
U is independently hydrogen, acetyl, or benzoyl;
X₁ is A or K;
X₄ is L or αMeL;
X₆ is Z₁, Z₂ or T;
X₇ is Z₁, Z₂ or A;
X₈ is Z₁, Z₂ or V;
X₉ is Z₁, Z₂ or L;
X₁₀ is Z₁, Z₂ or G;
X₁₁ is Z₁, Z₂, homoR, Orn or R;
X₁₂ is Z₁, Z₂ or L;
X₁₃ is Z₁, Z₂ or S;
X₁₄ is Z₁, Z₂ or A;
X₁₅ is Z₁, Z₂ or E;
X₁₆ is Z₁, Z₂ or L;
X₁₇ is Z₁, Z₂ or H;
X₁₈ is Z₁, Z₂, K, αMeK or Orn;
X₁₉ is Z₁, Z₂ or L;
X₂₀ is Z₁, Z₂ or Aib;
X₂₂ is Y or αMeF;
X₂₄ is R, N-Me-R, homoR, norR, Q or r;
X₃₂ is P, Hyp, cis-P(4-NH₂), trans-P(4-NH₂) or p,
wherein the structure represented by formula (I) comprises two amino acids Z₁ or two amino acids Z₂, sites of the two amino acids Z₁ or two amino acids Z₂ are Xᵢ and Xᵢ₊₇, respectively, where i is any integer between 6 and 13,
wherein each amino acid Z₁ is independently selected from C, c, αMeC, HoC, Hoc, Pen or N-Me-C, and
wherein each amino acid Z₂ is independently selected from K, k, Dap, Dab, Orn, HomoK, N-Me-K, N-Me-k, αMeK or αMek.

2. The polypeptide, derivative thereof, or pharmaceutically acceptable salt thereof according to claim 1, wherein the polypeptide, derivative thereof, or pharmaceutically acceptable salt thereof satisfies one or more of the following conditions:
1) the sites of the two amino acids Z₁ or two amino acids Z₂ are one of the following groups: X₆ and X₁₃, X₇ and X₁₄, X₈ and X₁₅, X₉ and X₁₆, X₁₀ and X₁₇, X₁₁ and X₁₈, X₁₂ and X₁₉, or X₁₃ and X₂₀;
2) the two amino acids Z₁ or two amino acids Z₂ are independently selected from K, c or C;
3) X₁ is A or K, or
X₁ is A;
4) X₄ is L;
5) X₆ is K, c, C or T, or
X₆ is C or T, or
X₆ is T;
6) X₇ is K, c, C or A, or
X₇ is C or A, or
X₇ is A;
7) X₈ is C, V, c, HoC, Pen, αMeC, N-Me-C, K, Orn, k or Dab, or
X₈ is K, c, C or V, or
X₈ is C, V or K, or
X₈ is C or V, or
X₈ is V;
8) X₉ is K, c, C or L, or
X₉ is C or L, or
X₉ is L;
10) X₁₀ is K, c, C or G, or
X₁₀ is C or G, or
X₁₀ is G;
11) X₁₁ is K, c, C, homoR, Orn or R, or
X₁₁ is K, C, homoR, Orn or R, or
X₁₁ is K, C, homoR or R, or
X₁₁ is C, homoR or R, or
X₁₁ is homoR or R;
12) X₁₂ is K, c, C or L, or
X₁₂ is C or L;
X₁₂ is L;
13) X₁₃ is K, c, C or S, or
X₁₃ is C or S, or
X₁₃ is S;
14) X₁₄ is K, c, C or A, or
X₁₄ is C or A, or
X₁₄ is A;
15) X₁₅ is C, E, c, HoC, Pen, αMeC, αMeK, N-Me-C, K, Orn, k or Dab, or
X₁₅ is K, c, C or E, or
X₁₅ is K, C or E, or
X₁₅ is C or E, or
X₁₅ is C;
16) X₁₆ is K, c, C or L, or
X₁₆ is C or L, or
X₁₆ is L;
17) X₁₇ is K, c, C or H, or
X₁₇ is C or H, or
X₁₇ is H;
18) X₁₈ is c, K, C, k, αMeK or Orn, or
X₁₈ is C, K, αMeK or Orn, or
X₁₈ is C, K or αMeK, or
X₁₈ is K or αMeK;
19) X₁₉ is K, c, C or L, or
X₁₉ is C or L, or
X₁₉ is L;
20) X₂₀ is K, c, C or Aib, or
X₂₀ is C or Aib, or
X₂₀ is Aib;
21) X₂₂ is Y;
22) X₂₃ is P;
23) X₂₄ is R, N-Me-R, homoR, norR, r or Q, or
X₂₄ is R, N-Me-R, homoR, norR or Q, or
X₂₄ is R, N-Me-R or Q;
24) X₂₉ is A; or
25) X₃₂ is P or Hyp.

3. The polypeptide, derivative thereof, or pharmaceutically acceptable salt thereof according to claim 1, wherein the polypeptide, derivative thereof, or pharmaceutically acceptable salt thereof satisfies one or more of the following conditions:
1) the sites of the two amino acids Z₁ or two amino acids Z₂ are X₈ and X₁₅, respectively;
2) the structure represented by formula (I) has one of the following structures:
U—X₁SHX₄STX₇X₈LGX₁₁LX₁₃X₁₄X₁₅LHX₁₈LX₂₀DX₂₂X₂₃X₂₄TDVGAGSX₃₂-NH₂ (Ia),
U—X₁SHX₄STAX₈LGX₁₁LSAX₁₅LHX₁₈L-Aib-DX₂₂PX₂₄TDVGAGSX₃₂-NH₂ (Ib),
U-ASHX₄STX₇X₈LGX₁₁X₁₂X₁₃X₁₄X₁₅LX₁₇X₁₈X₁₉X₂₀DX₂₂PX₂₄TDVGAGSX₃₂-NH₂ (II),
U-ASHX₄STAX₈LGX₁₁LX₁₃AX₁₅LHX₁₈LX₂₀DX₂₂PX₂₄TDVGAGSX₃₂-NH₂ (IIa),
U-ASHLSTAX₈LGX₁₁LSAX₁₅LHX₁₈L-Aib-DYPX₂₄TDVGAGSX₃₂—NH₂ (IIb), and
U-ASHLSTACLGX₁₁LSACLHX₁₈L-Aib-DYPX₂₄TDVGAGSX₃₂-NH₂ (III); or
3) the polypeptide, derivative thereof, or pharmaceutically acceptable salt thereof further comprises a modifying group, the modifying group being bound to the two amino acids Z₁ or the two amino acids Z₂ in the polypeptide or derivative thereof represented by formula (I).

4. The polypeptide, derivative thereof, or pharmaceutically acceptable salt thereof according to claim 1, wherein the structure represented by formula (I) has one of the following structures:
U-ASHLSCAVLGRLCAELHKL-Aib-DYPRTDVGAGSP-NH₂;
U-ASHLSTCVLGRLSCELHKL-Aib-DYPRTDVGAGSP-NH₂;
U-ASHLSTACLGRLSACLHKL-Aib-DYPRTDVGAGSP-NH₂;
U-ASHLSTAVCGRLSAECHKL-Aib-DYPRTDVGAGSP-NH₂;
U-ASHLSTAVLCRLSAELCKL-Aib-DYPRTDVGAGSP-NH₂;
U-ASHLSTAVLGCLSAELHCL-Aib-DYPRTDVGAGSP-NH₂;
U-ASHLSTAVLGRCSAELHKC-Aib-DYPRTDVGAGSP-NH₂;
U-ASSHLSTAVLGRLCAELHKLCDYPRTDVGAGSP-NH₂;
U-ASHLSTACLGRLSACLH-Orn-L-Aib-D-(αMeF)-PRTDVGAGS-Hyp-NH₂;
U-ASH-(αMeL)-STACLGRLSACLHKL-Aib-D-(αMeF)—P—(N-Me-R)-TDVGAGSP-NH₂;
U-ASHLSTACLGRLSACLH-(αMeK)-L-Aib-DYP-(N-Me-R)-TDVGAGS-Hyp-NH₂;
U-ASHLSTACLG-homoR-LSACLH-(αMeK)-L-Aib-DYP-(N-Me-R)-TDVGAGSP-NH₂;
U-ASHLSTACLGRLSACLH-(αMeK)-L-Aib-DYP-homoR-TDVGAGS-Hyp-NH₂;
U-ASHLSTACLGRLSACLH-(αMeK)-L-Aib-DYP-norR-TDVGAGS-Hyp-NH₂;
U-ASHLSTACLGRLSACLH-(αMeK)-L-Aib-DYPQTDVGAGS-Hyp-NH₂;
U-ASHLSTACLGRLSACLH-(αMeK)-L-Aib-DYP-r-TDVGAGS-Hyp-NH₂;
U-ASHLSTAVLGCLSAELHCL-Aib-DYPQTDVGAGS-Hyp-NH₂;
U-KSHLSTACLGRLSACLH-(αMeK)-L-Aib-DYP-(N-Me-R)-TDVGAGS-Hyp-NH₂;
U—KS HLSTAVLGCLSAELHCL-Aib-DYPQTDVGAGS-Hyp-NH₂;
U-KSHLSTA-c-LGRLSA-c-LH-(αMeK)-L-Aib-DYPQTDVGAGS-Hyp-NH₂;
U-ASHLSTACLGRLSACLH-(αMeK)-L-Aib-DYPQTDVGAGS-(cis-P(4-NH₂))—NH₂;
U-ASHLSTACLGRLSACLH-(αMeK)-L-Aib-DYPQTDVGAGS-(trans-P(4-NH₂))—NH₂;
U-ASHLSTACLGRLSACLH-(αMeK)-L-Aib-DYPQTDVGAGS-p-NH₂;
U-ASHLSTACLG-homoR-LSACLH-(αMeK)-L-Aib-DYPQTDVGAGS-Hyp-NH₂;
U-ASHLSTACLG-Orn-LSACLH-(αMeK)-L-Aib-DYPQTDVGAGS-Hyp-NH₂;
U-ASHLSTA-HoC-LGRLSA-HoC-LH-(αMeK)-L-Aib-DYPQTDVGAGS-Hyp-NH₂;
U-ASHLSTA-Pen-LGRLSA-Pen-LH-(αMeK)-L-Aib-DYPQTDVGAGS-Hyp-NH₂;
U-ASHLSTA-(αMeC)-LGRLSA-(αMeC)-LH-(αMeK)-L-Aib-DYPQTDVGAGS-Hyp-NH₂;
U-ASHLSTACLGRLSACLH-(αMeK)-L-Aib-DYPQTDVGAGS-Hyp-NH₂;
U-ASHLSTA-(N-Me-C)-LGRLSA-(N-Me-C)-LH-(αMeK)-L-Aib-DYPQTDVGAGS-Hyp-NH₂;
U-ASHLSTAKLGRLSAKLH-(αMeK)-L-Aib-DYPQTDVGAGS-Hyp-NH₂;
U-ASHLSTAVLGKLSAELHKL-Aib-DYPQTDVGAGS-Hyp-NH₂;
U-ASHLSTA-Orn-LGRLSA-Orn-LH-(αMeK)-L-Aib-DYPQTDVGAGS-Hyp-NH₂; and
U-ASHLSTA-Dab-LGRLSA-Dab-LH-(αMeK)-L-Aib-DYPQTDVGAGS-Hyp-NH₂,
where U is independently selected from H, acetyl or benzoyl.

5. The polypeptide, derivative thereof, or pharmaceutically acceptable salt thereof according to claim 3, wherein:
the modifying group is bound to —SH on a side chain of the amino acid Z₁ via a sulfur-carbon bond; or
the modifying group is bound to —NH₂ on a side chain of the amino acid Z₂ via an amide bond.

6. The polypeptide, derivative thereof, or pharmaceutically acceptable salt thereof according to claim 5, wherein the modifying group has a structure represented by formula (IV): 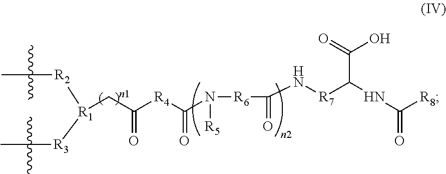
wherein:
R₁ is C, N, —C₃₋₁₀ heteroalkylene, —C₃₋₁₀ arylene or —C₃₋₁₀ heteroarylene;
R₂ and R₃ are each independently —C₁₋₆ alkylene- optionally substituted with one or more R₁ₐ, —NH—C(O)—C₁₋₆ alkylene- optionally substituted with one or more R₁ₐ, or —C₁₋₆ alkylene-NH—C(O)—C₁₋₆ alkylene- optionally substituted with one or more R₁ₐ, wherein each of the one or more R₁ₐ is independently halogen, —OH, —SH, —NH₂, —NO₂, —CN, phenyl, —C₁₋₆ alkyl, —C₁₋₆ halogenated alkyl or —C₁₋₆ alkoxyl;
R₄ is absent, or —C₁₋₆ alkylene-NH—C(O)—C₁₋₆ alkylene- optionally substituted with one or more R₂ₐ, wherein each of the one or more R₂ₐ is independently halogen, —OH, —SH, —NH₂, —NO₂, —CN, phenyl, —C₁₋₆ alkyl, —C₁₋₆ halogenated alkyl or —C₁₋₆ alkoxyl;
R₅ is H, —C₁₋₆ alkoxyl, or —C₁₋₆ alkyl, wherein the —C₁₋₆ alkyl and the —C₁₋₆ alkoxyl are each independently and optionally substituted with one or more halogens, —OH, —C(O)OH, —C(O)—, —SH, —NH₂, —NO₂, or —CN;
R₆ is —C₁₋₆ alkylene- optionally substituted with one or more R₃ₐ, or —(C₁₋₃ alkylene-O)ₘ₁—C₁₋₆ alkylene- optionally substituted with one or more R₃ₐ, wherein each of the one or more R₃ₐ is independently halogen, —OH, —SH, —NH₂, —NO₂, —CN, phenyl, —C₁₋₆ alkyl, —C₁₋₆ halogenated alkyl or —C₁₋₆ alkoxyl;
R₇ is —C₁₋₆ alkylene optionally substituted with one or more R₄ₐ, wherein each of the one or more R₄ₐ is independently halogen, —OH, —SH, —NH₂, —NO₂, —CN, phenyl, —C₁₋₆ alkyl, —C₁₋₆ halogenated alkyl or —C₁₋₆ alkoxyl;
R₈ is —C₁₀₋₂₀ alkyl optionally substituted with one or more R₅ₐ, —C₁₀₋₂₀ alkylene-R₉ optionally substituted with one or more R₅ₐ, —C₅₋₁₀ alkylene-O—C₃₋₁₀ arylene-R₉ optionally substituted with one or more R₅ₐ, or —C₅₋₁₀ alkylene-O—C₃₋₁₀ heteroarylene-R₉ optionally substituted with one or more R₅ₐ, wherein each of the one or more R₅ₐ is independently halogen, —OH, —SH, —NH₂, —NO₂, —CN, phenyl, —C₁₋₆ alkyl, —C₁₋₆ halogenated alkyl or —C₁₋₆ alkoxyl;
R₉ is —COOH, —C₃₋₇ heteroaryl, —S(O)₂OH or —PO(OH)₂;
m1 is any integer from 1 to 6;
n1 is any integer from 0 to 6; and
n2 is any integer from 1 to 10.

7. The polypeptide, derivative thereof, or pharmaceutically acceptable salt thereof according to claim 6, wherein the polypeptide, derivative thereof, or pharmaceutically acceptable salt thereof satisfies one or more of the following conditions:
1) R₁ is C, N, —C₅₋₇ heteroalkylene, —C₅₋₇ arylene or —C₅₋₇ heteroarylene;
2) R₂ and R₃ are each independently —C₁₋₆ alkylene- or —C₁₋₆ alkylene-NH—C(O)—C₁₋₆ alkylene-;
3) n1 is independently 0, 1, 2, 3, 4 or 5;
4) R₄ is absent, or —C₁₋₆ alkylene-NH—C(O)—C₁₋₆ alkylene-;
5) R₅ is H, —C₁₋₃ alkoxyl or —C₁₋₃ alkyl;
6) R₆ is —C₁₋₆ alkylene-, or —(C₁₋₃ alkylene-O)ₘ₁—C₁₋₆ alkylene-;
7) m1 is 2, 3, 4 or 5;
8) R₇ is —C₁₋₆ alkylene-;
9) R₈ is —C₁₀₋₂₀ alkyl, —C₁₀₋₂₀ alkylene-R₉, —C₅₋₁₀ alkylene-O—C₃₋₁₀ arylene-R₉, or —C₅₋₁₀ alkylene-O—C₃₋₁₀ heteroarylene-R₉;
10) R₉ is —COOH, —C₅₋₆ heteroaryl, —S(O)₂OH, or —PO(OH)₂;
11) n2 is 1, 2, or 3; or n2 is 3, 4, 5, or 6; or
12) the polypeptide or derivative thereof represented by formula (I) comprises two amino acids Z₁, and the modifying group has a structure represented by formula (IV).

8. The polypeptide, derivative thereof, or pharmaceutically acceptable salt thereof according to claim 6, wherein the polypeptide, derivative thereof, or pharmaceutically acceptable salt thereof satisfies one or more of the following conditions:
1) the modifying group represented by formula (IV) has a structure represented by formula (IVa): 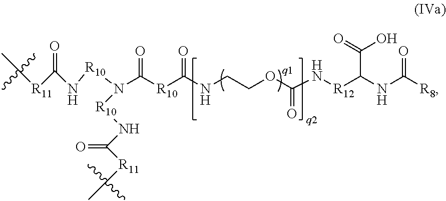
wherein:
each R₁₀ and each R₁₁ are independently —C₀₋₃ alkylene-,
R₁₂ is —C₁₋₆ alkylene-,
R₈ is —C₁₀₋₂₁ alkyl or —C₁₀₋₂₁ alkylene-COOH, and preferably, —C₁₄₋₁₉ alkyl or —C₁₀₋₁₈ alkylene-COOH,
q1 is 1, 2, 3 or 4, and
q2 is 1, 2 or 3;
2) the modifying group represented by formula (IV) has a structure represented by formula (IVb): 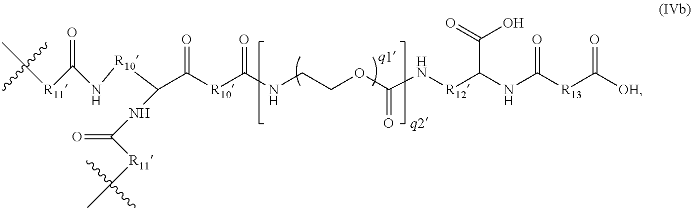
wherein:
each R₁₀′ and each R₁₁′ are each independently —C₀₋₃ alkylene-,
R₁₂′ is —C₁₋₆ alkylene-,
R₁₃ is —C₁₀₋₂₀ alkylene-, and preferably, —C₁₄₋₁₈ alkylene-,
q1′ is 1, 2, 3 or 4, and
q2′ is 1, 2 or 3; or
3) the modifying group represented by formula (IV) has a structure represented by formula (IVc): 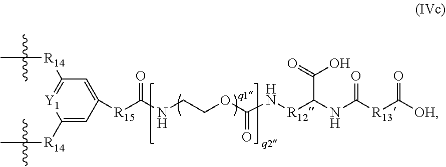
wherein:
each R₁₄ is independently —C₁₋₃ alkylene- or —NH—C(O)—C₁₋₃ alkylene-,
R₁₅ is —C₀₋₃ alkylene- or —C(O)—NH—C₁₋₃ alkylene-,
R₁₂″ is —C₁₋₆ alkylene-,
R₁₃′ is —C₁₀₋₂₀ alkylene-, and preferably, —C₁₄₋₁₈ alkylene-,
q1″ is 1, 2, 3 or 4,
q2″ is 1, 2 or 3, and
Y₁ is C or N.

9. The polypeptide, derivative thereof, or pharmaceutically acceptable salt thereof according to claim 6, wherein the polypeptide, derivative thereof, or pharmaceutically acceptable salt thereof satisfies one or more of the following conditions:
1) the modifying group represented by formula (IVa) has a structure represented by one of the following formulas: 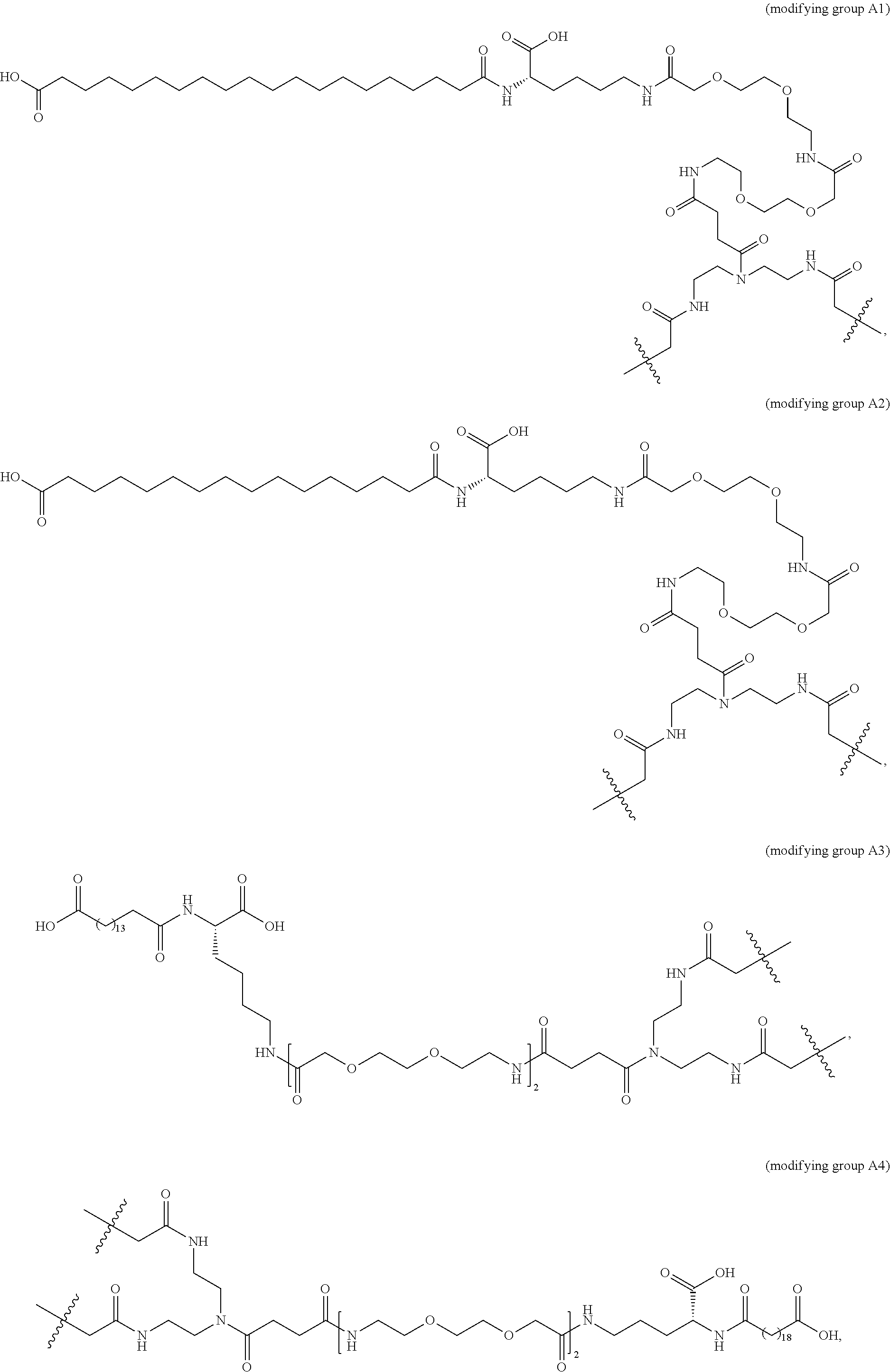 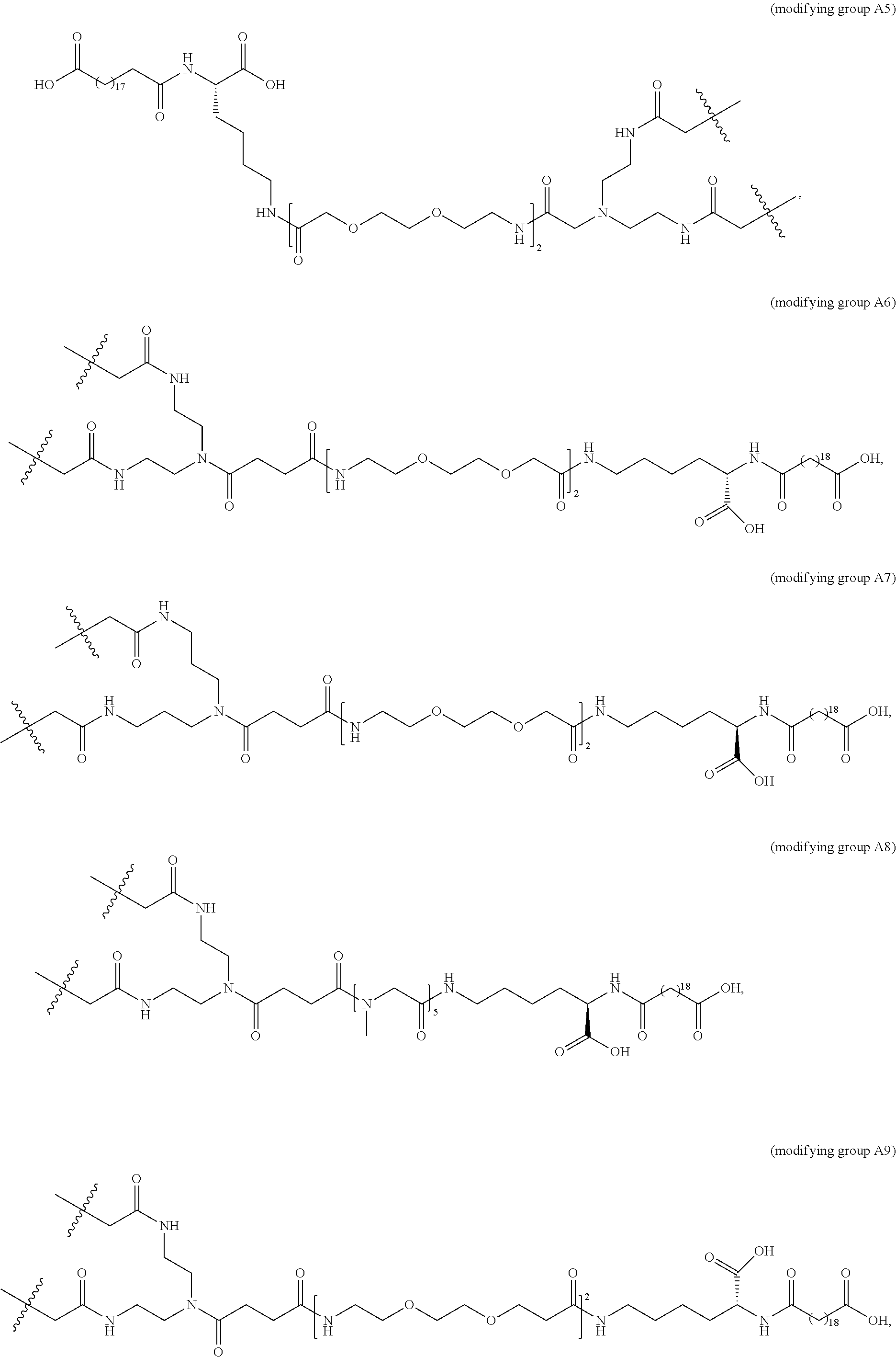 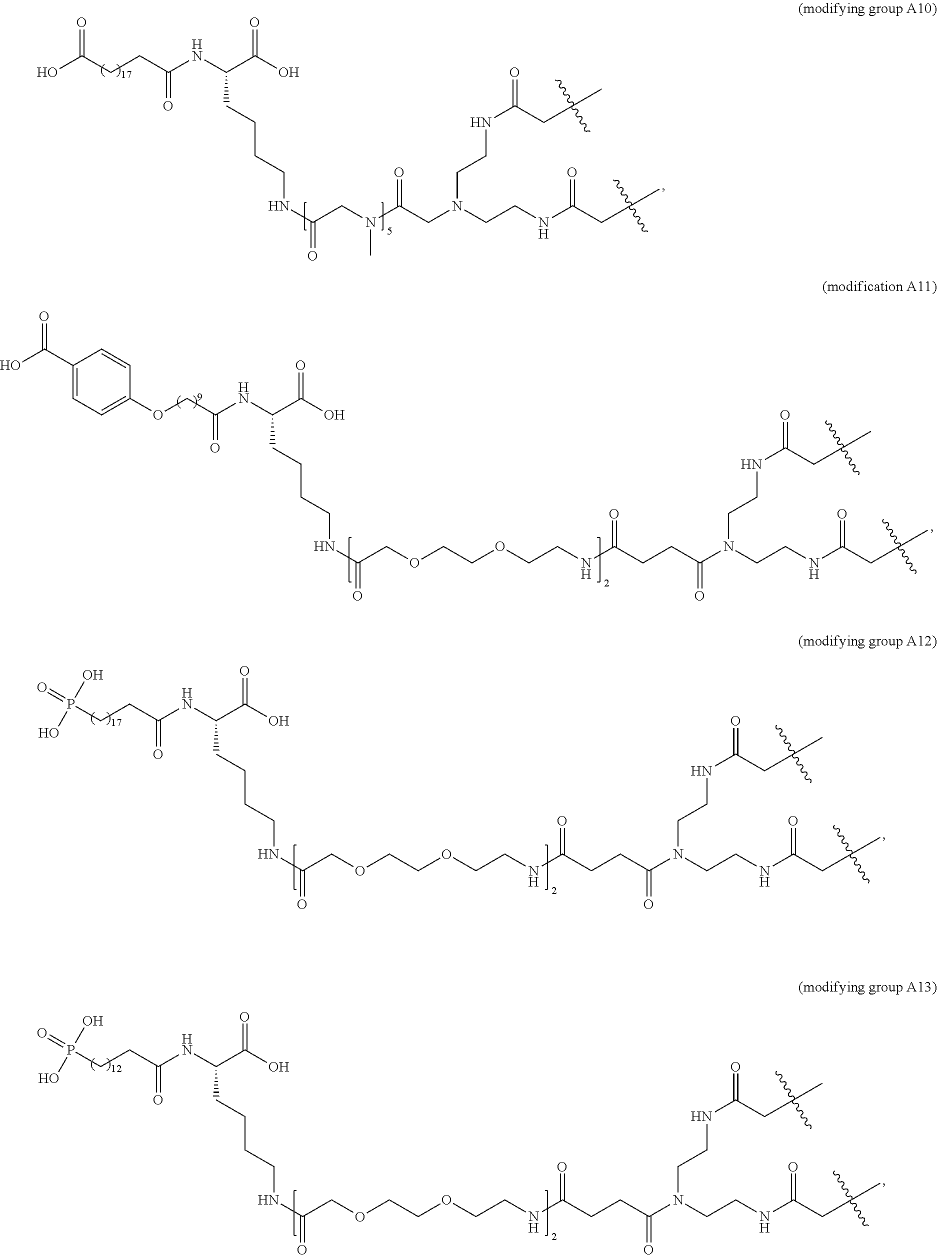 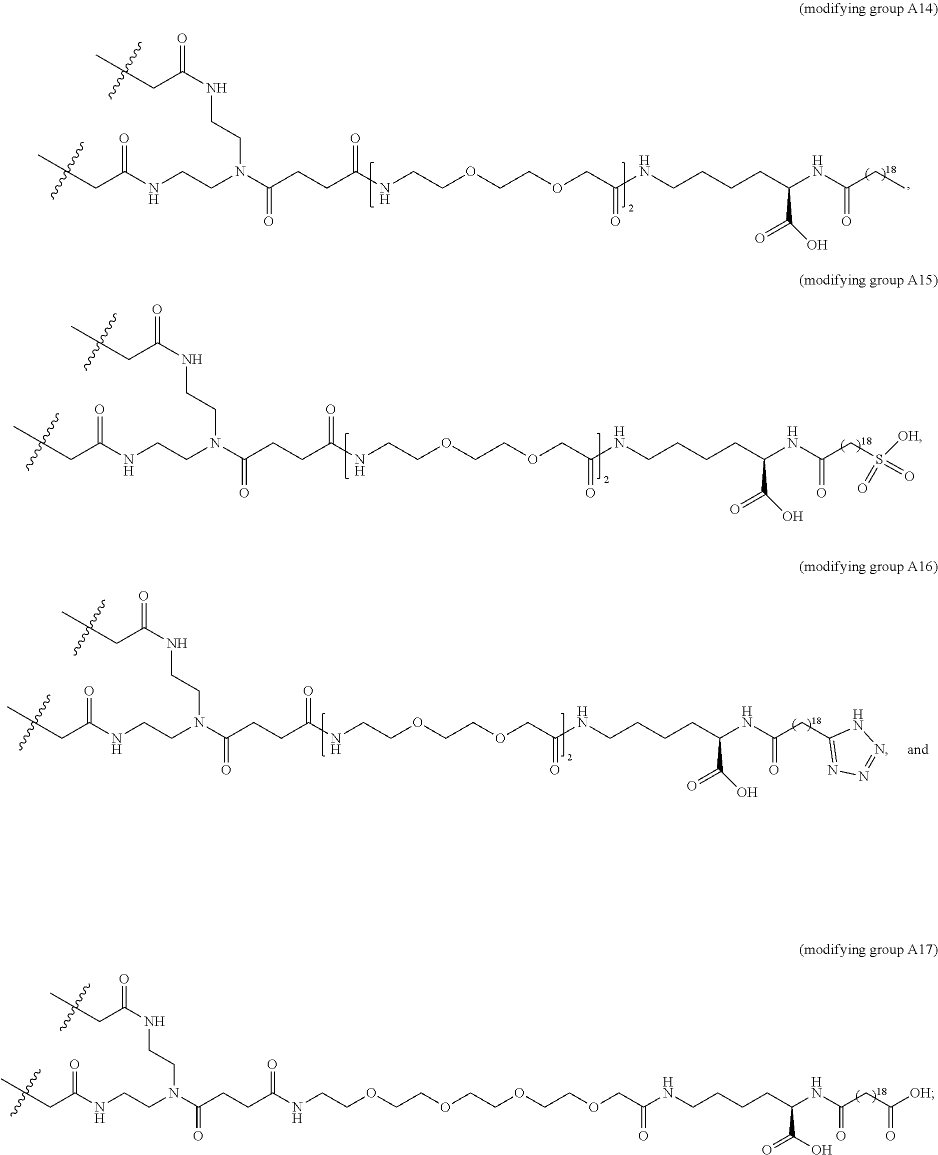
2) the modifying group represented by formula (IVb) has a structure represented by one of the following formulas: 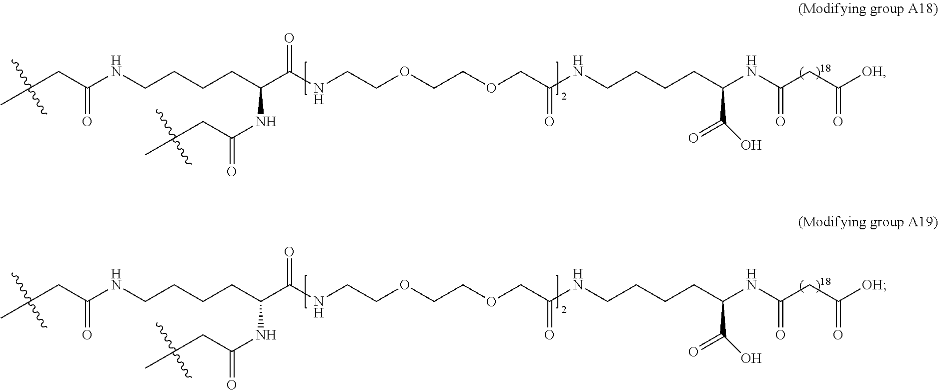
or
3) the modifying group represented by formula (IVc) has a structure represented by one of the following formulas:

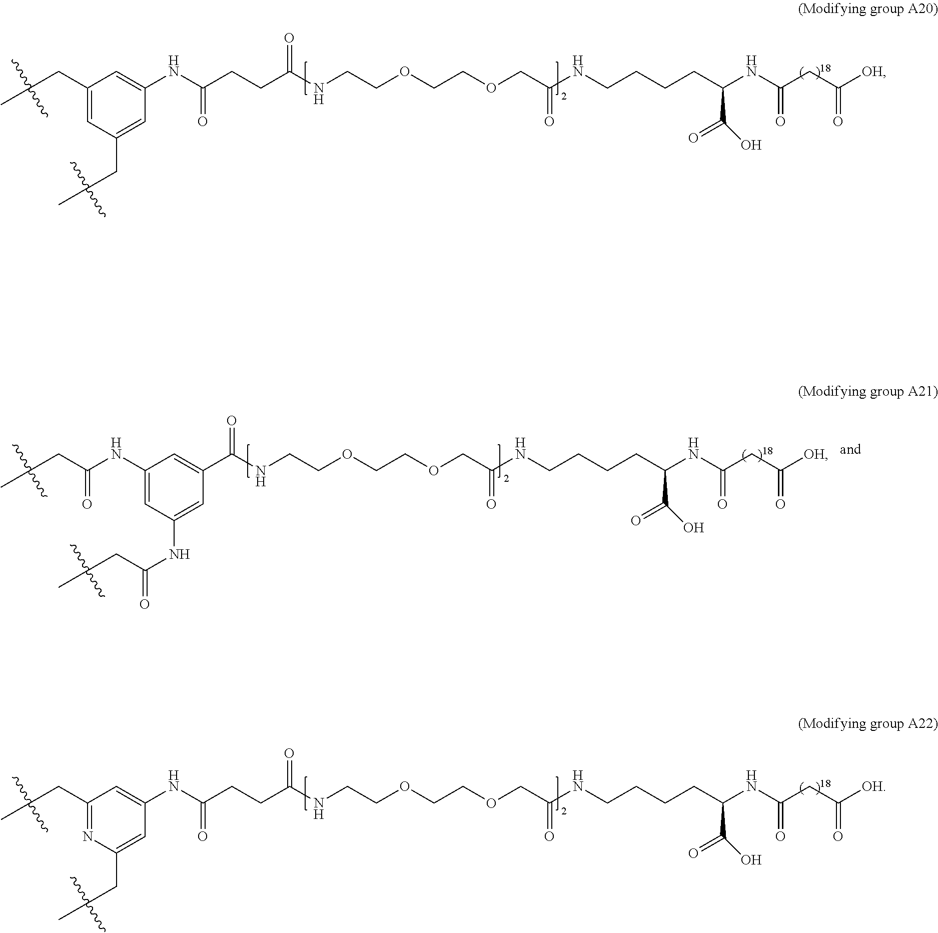

10. The polypeptide, derivative thereof, or pharmaceutically acceptable salt thereof according to claim 5, wherein the modifying group has a structure represented by formula (V): 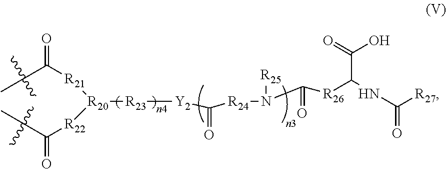
wherein:
R₂₀ is C, N, —C₃₋₁₀ arylene or —C₃₋₁₀ heteroarylene;
R₂₁ and R₂₂ are each independently absent, —C₁₋₆ alkylene- optionally substituted with one or more R_{1b}, or —C₁₋₆ alkoxylidene- optionally substituted with one or more R_{1b}, wherein each of the one or more R_{1b} is independently halogen, —OH, —SH, —NH₂, —NO₂, —CN, phenyl, —C₁₋₆ alkyl, —C₁₋₆ halogenated alkyl or —C₁₋₆ alkoxyl;
R₂₃ is absent, —C₁₋₆ alkylene- optionally substituted with one or more R_{2b}, —C(O)—C₁₋₆ alkylene- optionally substituted with one or more R_{2b}, —NH—C(O)—C₁₋₆ alkylene- optionally substituted with one or more R_{2b}, or —C₁₋₆ alkylene-C(O)—NH—C₁₋₆ alkylene- optionally substituted with one or more R_{2b}, wherein each of the one or more R_{2b} is independently halogen, —OH, —SH, —NH₂, —NO₂, —CN, phenyl, —C₁₋₆ alkyl, —C₁₋₆ halogenated alkyl or —C₁₋₆ alkoxyl;
R₂₄ is —C₁₋₆ alkylene- optionally substituted with one or more R_{3b}, or —(C₁₋₃ alkylene-O)ₘ₂—C₁₋₆ alkylene- optionally substituted with one or more R_{3b}, wherein each of the one or more R_{3b} is independently halogen, —OH, —SH, —NH₂, —NO₂, —CN, phenyl, —C₁₋₆ alkyl, —C₁₋₆ halogenated alkyl or —C₁₋₆ alkoxyl;
R₂₅ is H, —C₁₋₆ alkoxyl or —C₁₋₆ alkyl, wherein the —C₁₋₆ alkyl and the —C₁₋₆ alkoxyl are each independently and optionally substituted with one or more halogens, —OH, —C(O)OH, —C(O)—, —SH, —NH₂, —NO₂ or —CN;
R₂₆ is —C₁₋₆ alkylene- optionally substituted with one or more R_{4b}, wherein each of the one or more R_{4b} is independently halogen, —OH, —SH, —NH₂, —NO₂, —CN, phenyl, —C₁₋₆ alkyl, —C₁₋₆ halogenated alkyl or —C₁₋₆ alkoxyl;
R₂₇ is —C₁₀₋₂₀ alkyl optionally substituted with one or more R_{5b}, —C₁₀₋₂₀ alkylene-R₂₈ optionally substituted with one or more R_{5b}, —C₅₋₁₀ alkylene-O—C₃₋₁₀ arylene-R₂₈ optionally substituted with one or more R_{5b}, or —C₅₋₁₀ alkylene-O—C₃₋₁₀ heteroarylene-R₂₈ optionally substituted with one or more R_{5b}, wherein each of the one or more R_{5b} is independently halogen, —OH, —SH, —NH₂, —NO₂, —CN, phenyl, —C₁₋₆ alkyl, —C₁₋₆ halogenated alkyl or —C₁₋₆ alkoxyl;
R₂₈ is —COOH, —C₃₋₇ heteroaryl, —S(O)₂OH or —PO(OH)₂;
m2 is any integer from 1 to 6;
n3 is any integer from 1 to 10;
n3 is 0, 1 or 2; and
Y₂ is absent or NH.

11. The polypeptide, derivative thereof, or pharmaceutically acceptable salt thereof according to claim 10, wherein the polypeptide, derivative thereof or pharmaceutically acceptable salt thereof satisfies one or more of the following conditions:
1) R₂₀ is C, N, —C₅₋₇ arylene, or —C₅₋₇ heteroarylene;
2) R₂₁ and R₂₂ are each independently absent, —C₁₋₆ alkylene-, or —C₁₋₆ alkoxylidene-;
3) R₂₃ is absent, —C₁₋₆ alkylene-, —C₁₋₆ alkylene-C(O)—NH—C₁₋₆ alkylene-, or —C(O)—C₁₋₆ alkylene-;
4) R₂₄ is —C₁₋₆ alkylene-, or —(C₁₋₃ alkylene-O)ₘ2-C₁₋₆ alkylene-;
5) m2 is 2, 3, 4 or 5;
6) R₂₅ is H, —C₁₋₆ alkoxyl or —C₁₋₆ alkyl;
7) R₂₆ is —C₁₋₆ alkylene-;
8) R₂₇ is —C₁₀₋₂₀ alkyl, —C₁₀₋₂₀ alkylene-R₂₈, —C₅₋₁₀ alkylene-O—C₃₋₁₀ arylene-R₂₈, or —C₅₋₁₀ alkylene-O—C₃₋₁₀ heteroarylene-R₂₈;
9) R₂₈ is —COOH, —C₅₋₆ heteroaryl, —S(O)₂OH or —PO(OH)₂; or
10) p is 1, 2 or 3; or
p is 3, 4, 5 or 6.

12. The polypeptide, derivative thereof, or pharmaceutically acceptable salt thereof according to claim 10, wherein the polypeptide, derivative thereof or pharmaceutically acceptable salt thereof satisfies one or more of the following conditions:
1) the modifying group represented by formula (V) has a structure represented by formula (Va): 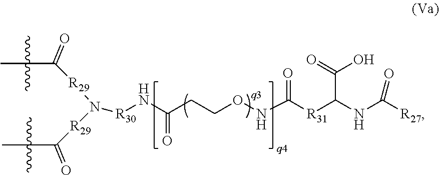
wherein:
each R₂₉ is independently —C₁₋₃ alkylene-,
R₃₀ is —C(O)—C₁₋₆ alkylene-, or —C₁₋₃ alkylene-C(O)—NH—C₁₋₃ alkylene-,
R₃₁ is —C₁₋₆ alkylene-,
q3 is 1, 2, 3 or 4, and
q4 is 1, 2 or 3;
2) the modifying group represented by formula (V) has a structure represented by formula (Vb): 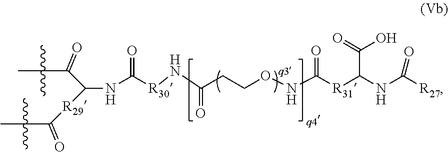
wherein:
each R₂₉′ is independently —C₁₋₃ alkylene-,
R₃₀′ is —C₁₋₆ alkylene-,
R₃₁′ is —C₁₋₆ alkylene-,
q3′ is 1, 2, 3 or 4, and preferably, 2, and
q4′ is 1, 2 or 3, and preferably, 2;
3) the modifying group represented by formula (V) has a structure represented by formula (Vc): 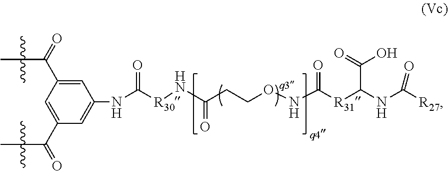
wherein:
R₃₀″ is —C₁₋₆ alkylene-,
R₃₁″ is —C₁₋₆ alkylene-,
q3 is 1, 2, 3 or 4, and
q4 is 1, 2 or 3; or
4) the polypeptide, or derivative thereof represented by formula (I) comprises the two amino acids Z₂, and the modifying group has a structure represented by formula (V).

13. The polypeptide, derivative thereof, or pharmaceutically acceptable salt thereof according to claim 12, wherein the polypeptide, derivative thereof, or pharmaceutically acceptable salt thereof satisfies one or more of the following conditions:
1) the modifying group represented by formula (Va) has a structure represented by one of the following formulas: 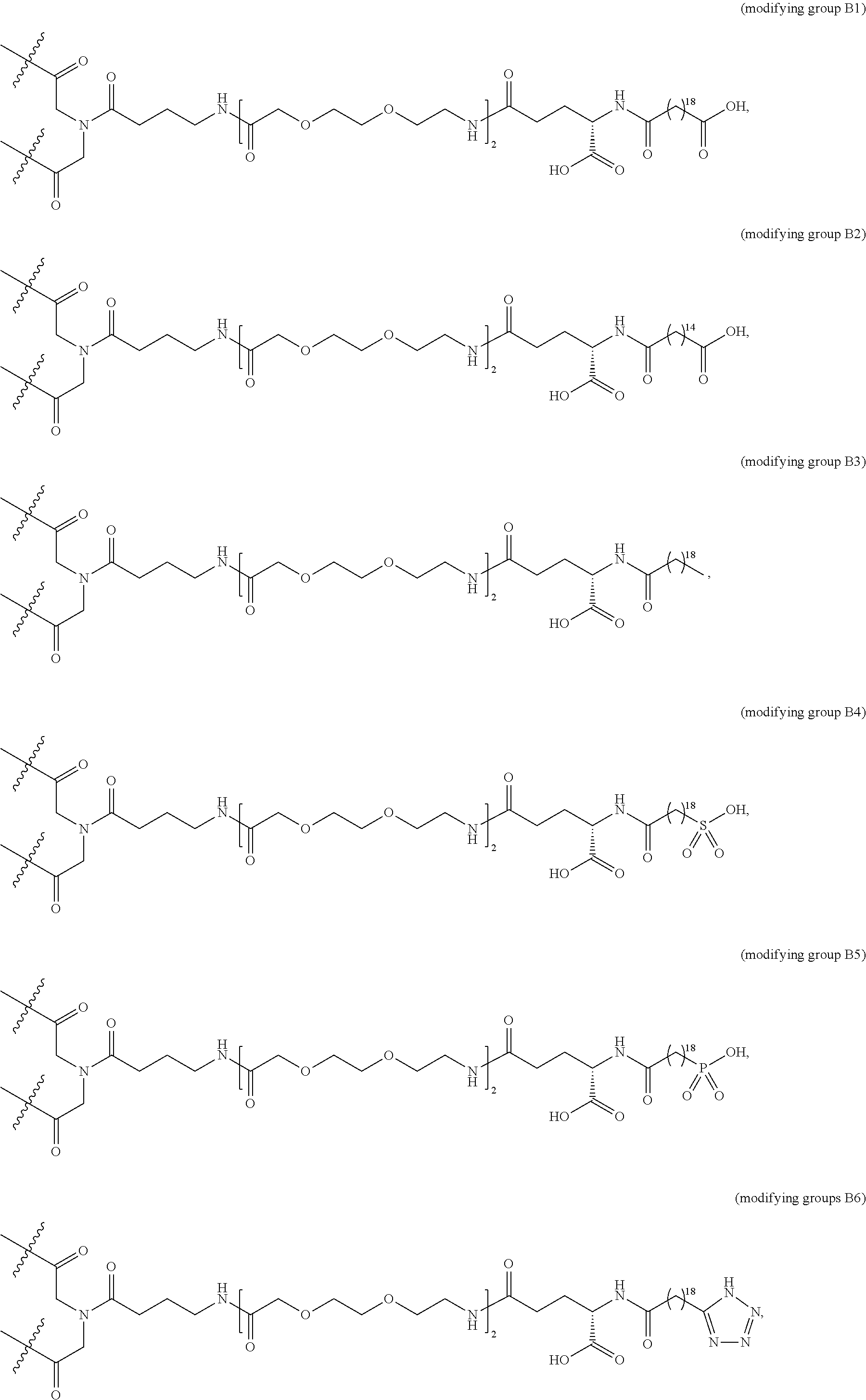 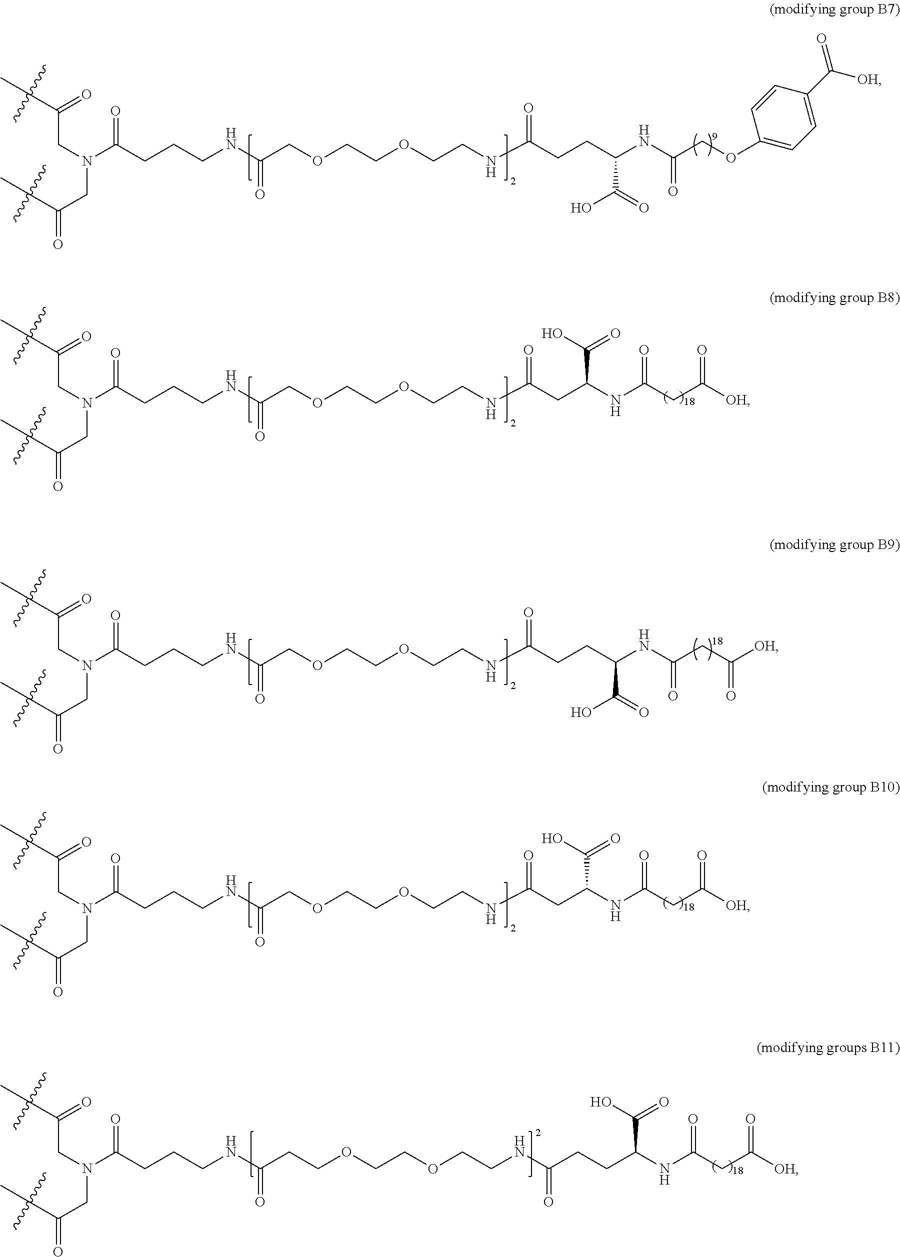 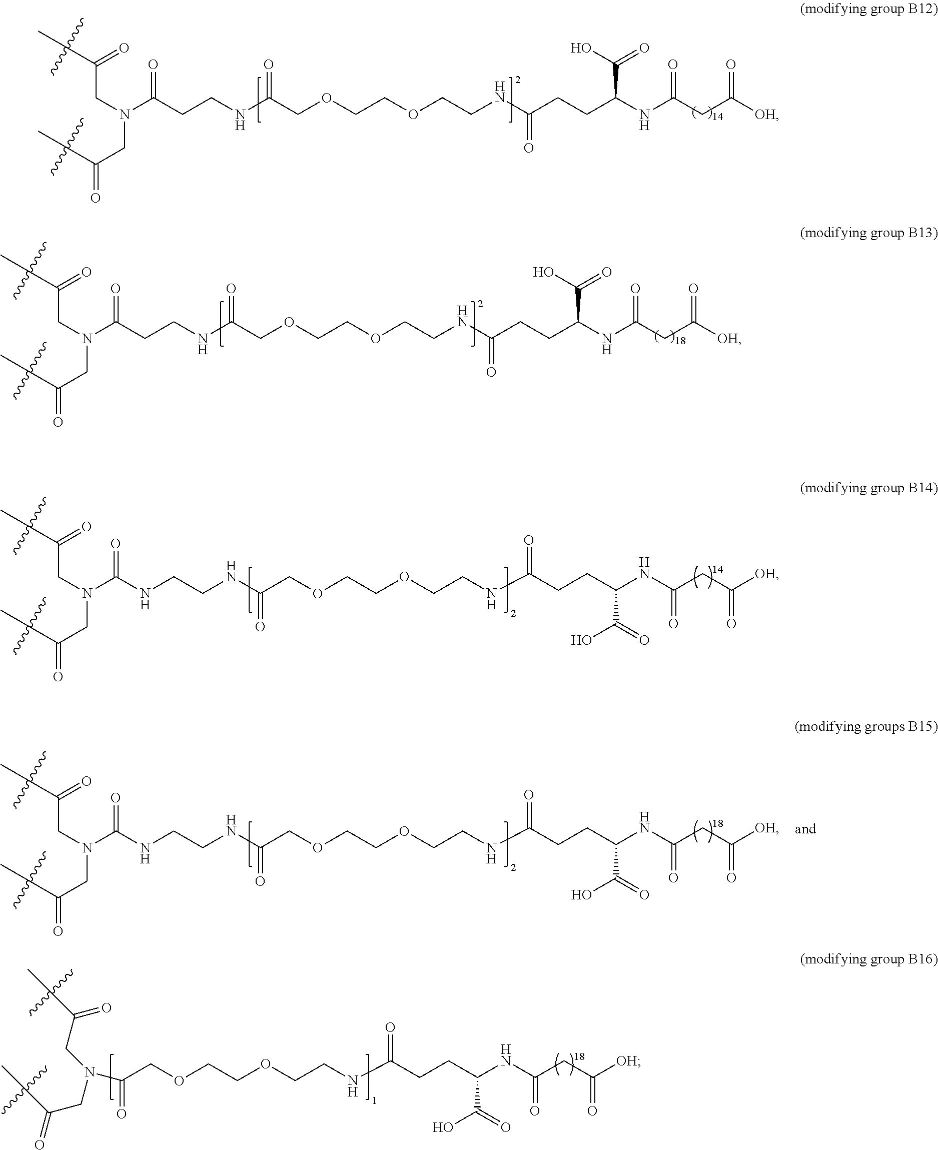
2) the modifying group represented by formula (Vb) has a structure represented by one of the following formulas: 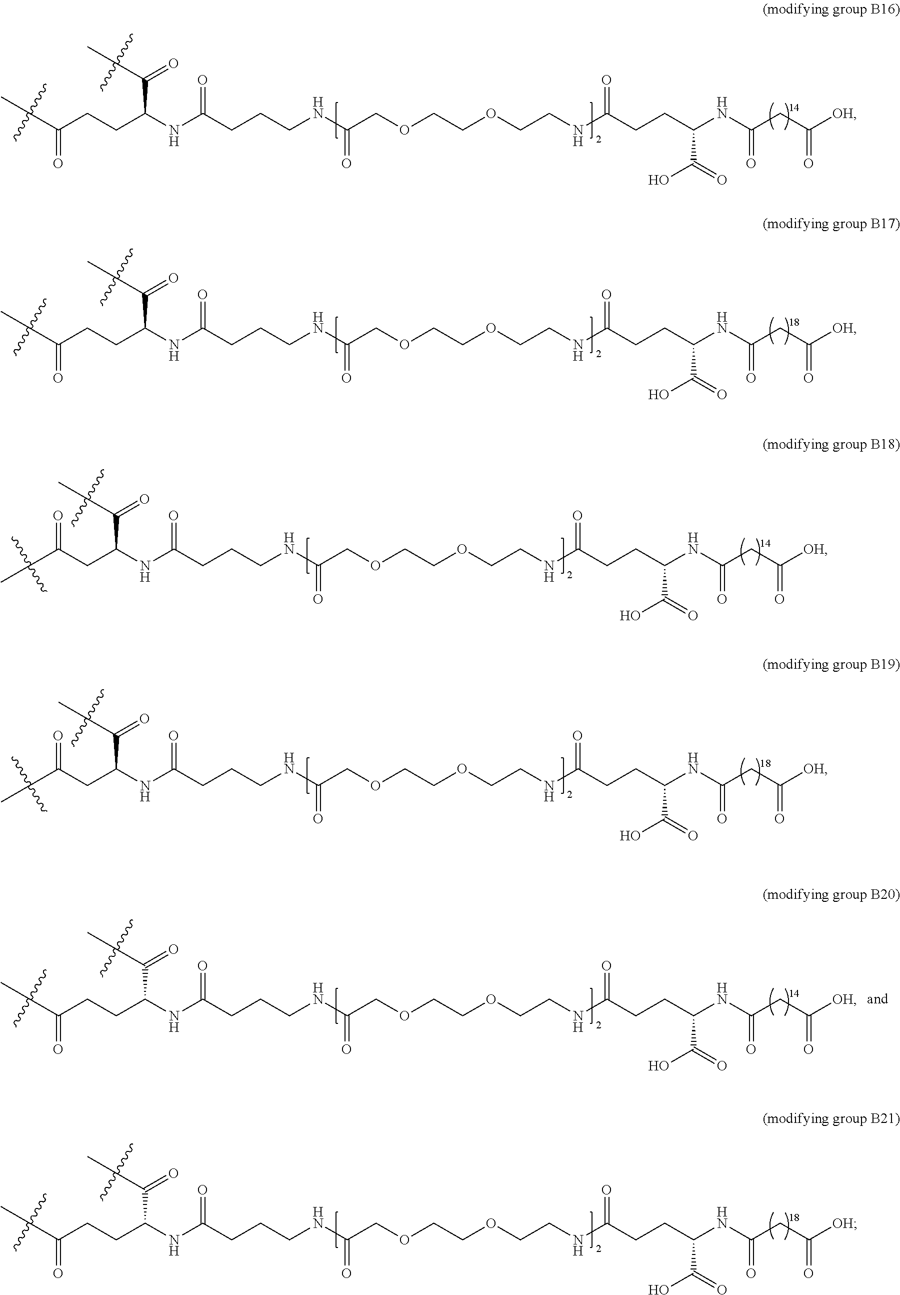
or
3) the modifying group represented by formula (Vc) has a structure represented by following formulas:

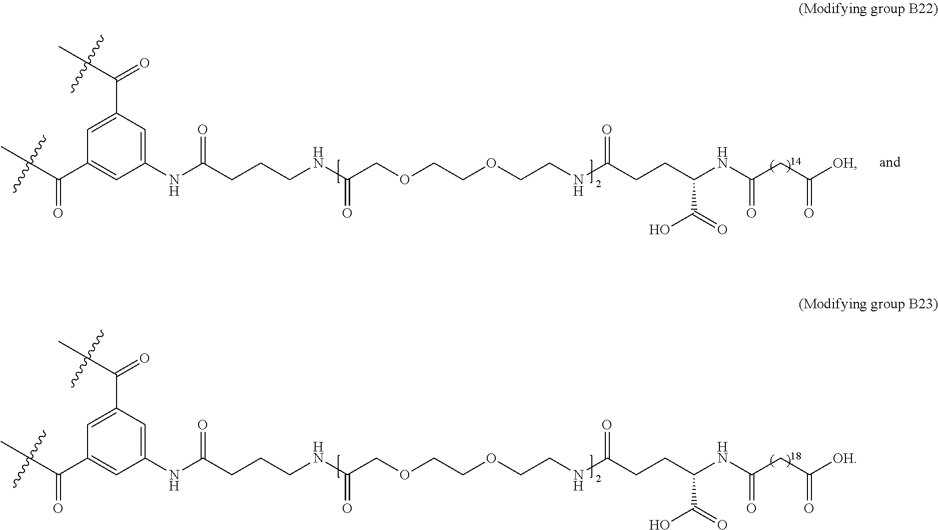

14. The polypeptide, derivative thereof, or pharmaceutically acceptable salt thereof according to claim 1, wherein the polypeptide derivative has a structure as shown in Table A or Table B:
TABLE A

| | | Binding sites | |
|---|---|---|---|
| Poly- | | (i, i + 7) of | |
| peptide | Amino acid sequence of | modifying | Modifying |
| name | polypeptide | groups | group |
| AMY-001 | Ac-ASHLSCAVLGRLCAELHKL-Aib- | 6, 13 | A1 |
| | DYPRTDVGAGSP-NH₂ | | |
| AMY-002 | Ac ASHLSTCVLGRLSCELHKL-Aib- | 7, 14 | A1 |
| | DYPRTDVGAGSP-NH₂ | | |
| AMY-003 | Ac-ASHLSTACLGRLSACLHKL-Aib- | 8, 15 | A1 |
| | DYPRTDVGAGSP-NH₂ | | |
| AMY-004 | Ac-ASHLSTAVCGRLSAECHKL-Aib- | 9, 16 | A1 |
| | DYPRTDVGAGSP-NH₂ | | |
| AMY-005 | Ac-ASHLSTAVLCRLSAELCKL-Aib- | 10, 17 | A1 |
| | DYPRTDVGAGSP-NH₂ | | |
| AMY-006 | Ac-ASHLSTAVLGCLSAELHCL-Aib- | 11, 18 | A1 |
| | DYPRTDVGAGSP-NH₂ | | |
| AMY-007 | Ac-ASHLSTAVLGRCSAELHKC-Aib- | 12, 19 | A1 |
| | DYPRTDVGAGSP-NH₂ | | |
| AMY-008 | Ac- | 13, 20 | A1 |
| | ASHLSTAVLGRLCAELHKLCDYPRTDVGA | | |
| | GSP-NH₂ | | |
| AMY-009 | Ac-ASHLSTACLGRLSACLH-Orn-L-Aib-D- | 8, 15 | A1 |
| | (αMeF)-PRTDVGAGS-Hyp-NH₂ | | |
| AMY-010 | Ac-ASH-(aMeK)-STACLGRLSACLHKL-Aib- | 8, 15 | A1 |
| | D-(αMeF)-P-(N-Me-R)-TDVGAGSP-NH₂ | | |
| AMY-011 | Ac-ASHLSTACLGRLSACLH-(αMeK)-L-Aib- | 8, 15 | A1 |
| | DYP-(N-Me-R)-TDVGAGS-Hyp-NH₂ | | |
| AMY-012 | Ac-ASHLSTACLG-homoR-LSACLH- | 8, 15 | A1 |
| | (αMeK)-L-Aib-DYP-(N-Me-R)- | | |
| | TDVGAGSP-NH₂ | | |
| AMY-013 | Ac-ASHLSTACLGRLSACLH-(αMeK)-L-Aib- | 8, 15 | A1 |
| | DYP-homoR-TDVGAGS-Hyp-NH₂ | | |
| AMY-014 | Ac-ASHLSTACLGRLSACLH-(αMeK)-L-Aib- | 8, 15 | A1 |
| | DYP-norR-TDVGAGS-Hyp-NH₂ | | |
| AMY-015 | Ac-ASHLSTACLGRLSACLH-(αMeK)-L-Aib- | 8, 15 | A1 |
| | DYPQTDVGAGS-Hyp-NH₂ | | |
| AMY-016 | Ac-ASHLSTACLGRLSACLH-(αMeK)-L-Aib- | 8, 15 | A12 |
| | DYPQTDVGAGS-Hyp-NH₂ | | |
| AMY-017 | Ac-ASHLSTACLGRLSACLH-(αMeK)-L-Aib- | 8, 15 | A5 |
| | DYPQTDVGAGS-Hyp-NH₂ | | |
| AMY-018 | Ac-ASHLSTACLGRLSACLH-(αMeK)-L-Aib- | 8, 15 | A4 |
| | DYPQTDVGAGS-Hyp-NH₂ | | |
| AMY-019 | Ac-ASHLSTACLGRLSACLH-(αMeK)-L-Aib- | 8, 15 | A1 |
| | DYP-r-TDVGAGS-Hyp-NH₂ | | |
| AMY-020 | Ac-ASHLSTAVLGCLSAELHCL-Aib- | 11, 18 | A1 |
| | DYPQTDVGAGS-Hyp-NH₂ | | |
| AMY-023 | Ac-KSHLSTACLGRLSACLH-(αMeK)-L-Aib- | 8, 15 | A1 |
| | DYP-(N-Me-R)-TDVGAGS-Hyp-NH₂ | | |
| AMY-024 | Ac-KSHLSTAVLGCLSAELHCL-Aib- | 11, 18 | A1 |
| | DYPQTDVGAGS-Hyp-NH₂ | | |
| AMY-025 | Ac-KSHLSTA-c-LGRLSA-c-LH-(αMeK)-L- | 8, 15 | A1 |
| | Aib-DYPQTDVGAGS-Hyp-NH₂ | | |
| AMY-026 | H-ASHLSTACLGRLSACLH-(αMeK)-L-Aib- | 8, 15 | A12 |
| | DYPQTDVGAGS-Hyp-NH₂ | | |
| AMY-027 | Ac-ASHLSTACLGRLSACLH-(αMeK)-L-Aib- | 8, 15 | A1 |
| | DYPQTDVGAGS-(cis-P(4-NH₂))-NH₂ | | |
| AMY-028 | Ac-ASHLSTACLGRLSACLH-(αMeK)-L-Aib- | 8, 15 | A1 |
| | DYPQTDVGAGS-(trans-P(4-NH₂))-NH₂ | | |
| AMY-029 | Ac-ASHLSTACLGRLSACLH-(αMeK)-L-Aib- | 8, 15 | A1 |
| | DYPQTDVGAGS-p-NH₂ | | |
| AMY-030 | Ac-ASHLSTACLG-homoR-LSACLH- | 8, 15 | A1 |
| | (αMeK)-L-Aib-DYPQTDVGAGS-Hyp-NH₂ | | |
| AMY-031 | Ac-ASHLSTACLG-Orn-LSACLH-(αMeK)-L- | 8, 15 | A1 |
| | Aib-DYPQTDVGAGS-Hyp-NH₂ | | |
| AMY-032 | Ac-ASHLSTA-HoC-LGRLSA-HoC-LH- | 8, 15 | A1 |
| | (αMeK)-L-Aib-DYPQTDVGAGS-Hyp-NH₂ | | |
| AMY-033 | Ac-ASHLSTA-Pen-LGRLSA-Pen-LH- | 8, 15 | A1 |
| | (αMeK)-L-Aib-DYPQTDVGAGS-Hyp-NH₂ | | |
| AMY-034 | Ac-ASHLSTA-(αMeC)-LGRLSA-(αMeC)- | 8, 15 | A1 |
| | LH-(αMeK)-L-Aib-DYPQTDVGAGS-Hyp- | | |
| | NH₂ | | |
| AMY-035 | Bzl-ASHLSTACLGRLSACLH-(αMeK)-L- | 8, 15 | A1 |
| | Aib-DYPQTDVGAGS-Hyp-NH₂ | | |
| AMY-036 | Ac-ASHLSTA-(N-Me-C)-LGRLSA-(N-Me- | 8, 15 | A1 |
| | C)-LH-(αMeK)-L-Aib-DYPQTDVGAGS- | | |
| | Hyp-NH₂ | | |

TABLE B

| | | Binding sites | |
|---|---|---|---|
| Poly- | | (i, i + 7) of | |
| peptide | Amino acid sequence of | modifying | Modifying |
| name | polypeptide | groups | group |
| AMY-021 | Ac-ASHLSTAKLGRLSAKLH-(αMeK)-L-Aib- | 8, 15 | B1 |
| | DYPQTDVGAGS-Hyp-NH₂ | | |
| AMY-022 | Ac-ASHLSTAVLGKLSAELHKL-Aib- | 11, 18 | B16 |
| | DYPQTDVGAGS-Hyp-NH₂ | | |
| AMY-037 | Ac-ASHLSTA-Orn-LGRLSA-Orn-LH-(αMeK)- | 8, 15 | B2 |
| | L-Aib-DYPQTDVGAGS-Hyp-NH₂ | | |
| AMY-038 | Ac-ASHLSTAkLGRLSAkLH-(αMeK)-L-Aib- | 8, 15 | B2 |
| | DYPQTDVGAGS-Hyp-NH₂ | | |
| AMY-039 | Ac-ASHLSTA-Dab-LGRLSA-Dab-LH-(αMeK)- | 8, 15 | B1 |
| | L-Aib-DYPQTDVGAGS-Hyp-NH₂ | | |
| AMY-040 | H-ASHLSTAVLGKLSAELHKL-Aib- | 11, 18 | B16 |
| | DYPQTDVGAGS-Hyp-NH₂ | | |
| AMY-041 | Bzl-ASHLSTAVLGKLSAELHKL-Aib- | 11, 18 | B16 |
| | DYPQTDVGAGS-Hyp-NH₂ | | |

15. A pharmaceutical composition, comprising:
the polypeptide, derivative thereof, or pharmaceutically acceptable salt thereof according to claim 1, and
optionally, a pharmaceutically acceptable excipient or carrier.

16. A combination of medicament or kit, comprising:
the polypeptide, derivative thereof, or pharmaceutically acceptable salt thereof according to claim 1, as a first active ingredient; and
a second active ingredient, wherein the second active ingredient comprises a medicament for preventing and/or treating diseases comprising amylin receptor- and/or calcitonin receptor-related diseases.

17. The combination of medicament or kit according to claim 16, wherein the combination of medicament or kit satisfies one or more of the following conditions:
1) the amylin receptor- and/or calcitonin receptor-related diseases comprise lipid metabolism disorders, glucose metabolism disorders, cardiovascular diseases, brain diseases, mental diseases, or neurological diseases;
preferably, the amylin receptor- and/or calcitonin receptor-related diseases comprise at least one of metabolic disorder-related diseases, bone-related diseases, cardiovascular diseases unrelated to metabolic diseases, alcohol or drug addiction-related symptoms or diseases, or neurodegenerative diseases;
preferably, the amylin receptor- and/or calcitonin receptor-related diseases comprise at least one of diabetes mellitus, hypertension, arteriosclerosis, hepatic cirrhosis, coronary heart disease, angina, myocardial infarction, inflammatory bowel diseases, dyspepsia, gastrointestinal ulcers, hyperglycemia, impaired glucose tolerance, X syndrome, cognitive impairment, stroke, dyslipidemia-related diseases comprising hyperlipidemia and dyslipidemia, metabolic syndromes, non-alcoholic steatohepatitis, non-alcoholic fatty liver diseases, abnormal body weight, obesity, fatty liver disease, diabetic nephrotic syndromes, diabetes mellitus-related renal fibrosis, hepatic fibrosis, Alzheimer's disease, or Parkinson's disease; and
preferably, the amylin receptor- and/or calcitonin receptor-related diseases comprise at least one of cardiovascular diseases, diabetes mellitus and/or obesity; or
2) the medicament is selected from at least one of adrenergic receptor blockers, HMG-CoA reductase inhibitors, angiotensin receptor antagonists, angiotensin-converting enzyme inhibitors, calcium channel blockers, endothelin antagonists, renin inhibitors, diuretics, aldosterone receptor blockers, endothelin receptor blockers, aldosterone synthase inhibitors, CETP inhibitors, relaxin, PCSK9 inhibitors, BNP and NEP inhibitors, GLP-1 analogs, insulin, sulfonylureas, biguanides, meglitazones, glucosidase inhibitors, DPP IV inhibitors, or SGLT2 inhibitors;
preferably, the medicament is selected from one of GLP-1 analogs, gliflozins, biguanides and acarbose;
preferably, the GLP-1 analogs comprise at least one of Semaglutide, Exenatide, Liraglutide, Dulaglutide, Tirzepatide and Retatrutdie;
preferably, the gliflozins comprise at least one of dapagliflozin and empagliflozin; and
preferably, the biguanides comprise metformin.

18. A method for treating or preventing amylin receptor- and/or calcitonin receptor-related diseases, the method comprising:
administrating to a subject a pharmaceutically acceptable amount of the polypeptide, derivative thereof, or pharmaceutically acceptable salt thereof according to claim 1.

19. The method according to claim 18, wherein the amylin receptor- and/or calcitonin receptor-related diseases comprise lipid metabolism disorders, glucose metabolism disorders, cardiovascular diseases, brain diseases, mental diseases, or neurological diseases;
preferably, the amylin receptor- and/or calcitonin receptor-related diseases comprise at least one of metabolic disorder-related diseases, bone-related diseases, cardiovascular diseases unrelated to metabolic diseases, alcohol or medicament addiction-related symptoms or diseases, or neurodegenerative diseases; and
preferably, the amylin receptor- and/or calcitonin receptor-related diseases comprise at least one of diabetes mellitus, hypertension, arteriosclerosis, hepatic cirrhosis, coronary heart disease, angina, myocardial infarction, inflammatory bowel diseases, dyspepsia, gastrointestinal ulcers, hyperglycemia, impaired glucose tolerance, X syndrome, cognitive impairment, stroke, dyslipidemia-related diseases comprising hyperlipidemia and dyslipidemia, metabolic syndromes, non-alcoholic steatohepatitis, non-alcoholic fatty liver diseases, abnormal body weight, obesity, fatty liver disease, diabetic nephrotic syndromes, diabetes mellitus-related renal fibrosis, hepatic fibrosis, Alzheimer's disease, or Parkinson's disease.


SEQUENCE LISTING (37 sequences)

SEQ ID NO: 1 - protein, 32 aa, synthetic construct
  MOD_RES 1: note = Acetylation
  MOD_RES 20: note = 2-Aminoisobutyric acid
  MOD_RES 32: note = Amino group
ASHLSCAVLGRLCAELHKLXDYPRTDVGAGSP

SEQ ID NO: 2 - protein, 32 aa, synthetic construct
  MOD_RES 1: note = Acetylation
  MOD_RES 20: note = 2-Aminoisobutyric acid
  MOD_RES 32: note = Amino group
ASHLSTCVLGRLSCELHKLXDYPRTDVGAGSP

SEQ ID NO: 3 - protein, 32 aa, synthetic construct
  MOD_RES 1: note = Acetylation
  MOD_RES 20: note = 2-Aminoisobutyric acid
  MOD_RES 32: note = Amino group
ASHLSTACLGRLSACLHKLXDYPRTDVGAGSP

SEQ ID NO: 4 - protein, 32 aa, synthetic construct
  MOD_RES 1: note = Acetylation
  MOD_RES 20: note = 2-Aminoisobutyric acid
  MOD_RES 32: note = Amino group
ASHLSTAVCGRLSAECHKLXDYPRTDVGAGSP

SEQ ID NO: 5 - protein, 32 aa, synthetic construct
  MOD_RES 1: note = Acetylation
  MOD_RES 20: note = 2-Aminoisobutyric acid
  MOD_RES 32: note = Amino group
ASHLSTAVLCRLSAELCKLXDYPRTDVGAGSP

SEQ ID NO: 6 - protein, 32 aa, synthetic construct
  MOD_RES 1: note = Acetylation
  MOD_RES 20: note = 2-Aminoisobutyric acid
  MOD_RES 32: note = Amino group
ASHLSTAVLGCLSAELHCLXDYPRTDVGAGSP

SEQ ID NO: 7 - protein, 32 aa, synthetic construct
  MOD_RES 1: note = Acetylation
  MOD_RES 32: note = Amino group
  MOD_RES 20: note = 2-Aminoisobutyric acid
ASHLSTAVLGRCSAELHKCXDYPRTDVGAGSP

SEQ ID NO: 8 - protein, 32 aa, synthetic construct
  MOD_RES 1: note = Acetylation
  MOD_RES 32: note = Amino group
ASHLSTAVLGRLCAELHKLCDYPRTDVGAGSP

SEQ ID NO: 9 - protein, 32 aa, synthetic construct
  MOD_RES 1: note = Acetylation
  MOD_RES 18: note = Ornithine
  MOD_RES 20: note = 2-Aminoisobutyric acid
  MOD_RES 22: note = alpha Methylphenylalanine
  MOD_RES 32: note = Hydroxyproline
  MOD_RES 32: note = Amino group
ASHLSTACLGRLSACLXXLXDXPRTDVGAGSX

SEQ ID NO: 10 - protein, 32 aa, synthetic construct
  MOD_RES 1: note = Acetylation
  MOD_RES 32: note = Amino group
  MOD_RES 4: note = alpha Methylleucine
  MOD_RES 20: note = 2-Aminoisobutyric acid
  MOD_RES 22: note = alpha Methylphenylalanine
  MOD_RES 24: note = N Methylarginine
ASXXSTACLGRLSACLHKLXDXPXTDVGAGSP

SEQ ID NO: 11 - protein, 32 aa, synthetic construct
  MOD_RES 1: note = Acetylation
  MOD_RES 32: note = Amino group
  MOD_RES 18: note = alpha Methyllysine
  MOD_RES 20: note = 2-Aminoisobutyric acid
  MOD_RES 24: note = N Methylarginine
  MOD_RES 32: note = Hydroxyproline
ASHLSTACLGRLSACLXXLXDYPXTDVGAGSX

SEQ ID NO: 12 - protein, 32 aa, synthetic construct
  MOD_RES 1: note = Acetylation
  MOD_RES 32: note = Amino group
  MOD_RES 11: note = Homoarginine
  MOD_RES 18: note = alpha Methyllysine
  MOD_RES 20: note = 2-Aminoisobutyric acid
  MOD_RES 24: note = N Methylarginine
ASHLSTACLGXLSACLXXLXDYPXTDVGAGSP

SEQ ID NO: 13 - protein, 32 aa, synthetic construct; identical to SEQ ID NO: 11
  MOD_RES 1: note = Acetylation
  MOD_RES 32: note = Amino group
  MOD_RES 18: note = alpha Methyllysine
  MOD_RES 20: note = 2-Aminoisobutyric acid
  MOD_RES 24: note = Homoarginine
  MOD_RES 32: note = Hydroxyproline

SEQ ID NO: 14 - protein, 32 aa, synthetic construct; identical to SEQ ID NO: 11
  MOD_RES 1: note = Acetylation
  MOD_RES 32: note = Amino group
  MOD_RES 18: note = alpha Methyllysine
  MOD_RES 20: note = 2-Aminoisobutyric acid
  MOD_RES 24: note = norarginine
  MOD_RES 32: note = Hydroxyproline

SEQ ID NO: 15 - protein, 32 aa, synthetic construct
  MOD_RES 1: note = Acetylation
  MOD_RES 32: note = Amino group
  MOD_RES 18: note = alpha Methyllysine
  MOD_RES 20: note = 2-Aminoisobutyric acid
  MOD_RES 32: note = Hydroxyproline
ASHLSTACLGRLSACLXXLXDYPQTDVGAGSX

SEQ ID NO: 16 - protein, 32 aa, synthetic construct
  MOD_RES 1: note = Acetylation
  MOD_RES 32: note = Amino group
  MOD_RES 18: note = alpha Methyllysine
  MOD_RES 20: note = 2-Aminoisobutyric acid
  MOD_RES 32: note = Hydroxyproline
ASHLSTACLGRLSACLXXLXDYPRTDVGAGSX

SEQ ID NO: 17 - protein, 32 aa, synthetic construct
  MOD_RES 1: note = Acetylation
  MOD_RES 32: note = Amino group
  MOD_RES 20: note = 2-Aminoisobutyric acid
  MOD_RES 32: note = Hydroxyproline
ASHLSTAVLGCLSAELHCLXDYPQTDVGAGSX

SEQ ID NO: 18 - protein, 32 aa, synthetic construct
  MOD_RES 1: note = Acetylation
  MOD_RES 32: note = Amino group
  MOD_RES 18: note = alpha Methyllysine
  MOD_RES 20: note = 2-Aminoisobutyric acid
  MOD_RES 24: note = N Methylarginine
  MOD_RES 32: note = Hydroxyproline
KSHLSTACLGRLSACLXXLXDYPXTDVGAGSX

SEQ ID NO: 19 - protein, 32 aa, synthetic construct
  MOD_RES 1: note = Acetylation
  MOD_RES 32: note = Amino group
  MOD_RES 20: note = 2-Aminoisobutyric acid
  MOD_RES 32: note = Hydroxyproline
KSHLSTAVLGCLSAELHCLXDYPQTDVGAGSX

SEQ ID NO: 20 - protein, 32 aa, synthetic construct
  MOD_RES 1: note = Acetylation
  MOD_RES 32: note = Amino group
  MOD_RES 18: note = alpha Methyllysine
  MOD_RES 20: note = 2-Aminoisobutyric acid
  MOD_RES 32: note = Hydroxyproline
KSHLSTACLGRLSACLXXLXDYPQTDVGAGSX

SEQ ID NO: 21 - protein, 32 aa, synthetic construct; identical to SEQ ID NO: 15
  MOD_RES 1: note = Hydrogen
  MOD_RES 32: note = Amino group
  MOD_RES 18: note = alpha Methyllysine
  MOD_RES 20: note = 2-Aminoisobutyric acid
  MOD_RES 32: note = Hydroxyproline

SEQ ID NO: 22 - protein, 32 aa, synthetic construct; identical to SEQ ID NO: 15
  MOD_RES 1: note = Acetylation
  MOD_RES 32: note = Amino group
  MOD_RES 18: note = alpha Methyllysine
  MOD_RES 20: note = 2-Aminoisobutyric acid
  MOD_RES 32: note = cis 4 Aminoproline

SEQ ID NO: 23 - protein, 32 aa, synthetic construct; identical to SEQ ID NO: 15
  MOD_RES 1: note = Acetylation
  MOD_RES 32: note = Amino group
  MOD_RES 18: note = alpha Methyllysine
  MOD_RES 20: note = 2-Aminoisobutyric acid
  MOD_RES 32: note = trans 4 Aminoproline

SEQ ID NO: 24 - protein, 32 aa, synthetic construct
  MOD_RES 1: note = Acetylation
  MOD_RES 32: note = Amino group
  MOD_RES 18: note = alpha Methyllysine
  MOD_RES 20: note = 2-Aminoisobutyric acid
ASHLSTACLGRLSACLXXLXDYPQTDVGAGSP

SEQ ID NO: 25 - protein, 32 aa, synthetic construct
  MOD_RES 1: note = Acetylation
  MOD_RES 32: note = Amino group
  MOD_RES 11: note = Homoarginine
  MOD_RES 18: note = alpha Methyllysine
  MOD_RES 20: note = 2-Aminoisobutyric acid
  MOD_RES 32: note = Hydroxyproline
ASHLSTACLGXLSACLXXLXDYPQTDVGAGSX

SEQ ID NO: 26 - protein, 32 aa, synthetic construct; identical to SEQ ID NO: 25
  MOD_RES 1: note = Acetylation
  MOD_RES 32: note = Amino group
  MOD_RES 11: note = Ornithine
  MOD_RES 18: note = alpha Methyllysine
  MOD_RES 20: note = 2-Aminoisobutyric acid
  MOD_RES 32: note = Hydroxyproline

SEQ ID NO: 27 - protein, 32 aa, synthetic construct
  MOD_RES 1: note = Acetylation
  MOD_RES 32: note = Amino group
  MOD_RES 8: note = Homocysteine
  MOD_RES 15: note = Homocysteine
  MOD_RES 18: note = alpha Methyllysine
  MOD_RES 20: note = 2-Aminoisobutyric acid
  MOD_RES 32: note = Hydroxyproline
ASHLSTAXLGRLSAXLXXLXDYPQTDVGAGSX

SEQ ID NO: 28 - protein, 32 aa, synthetic construct; identical to SEQ ID NO: 27
  MOD_RES 1: note = Acetylation
  MOD_RES 32: note = Amino group
  MOD_RES 8: note = Penicillamine
  MOD_RES 15: note = Penicillamine
  MOD_RES 18: note = alpha Methyllysine
  MOD_RES 20: note = 2-Aminoisobutyric acid
  MOD_RES 32: note = Hydroxyproline

SEQ ID NO: 29 - protein, 32 aa, synthetic construct; identical to SEQ ID NO: 27
  MOD_RES 1: note = Acetylation
  MOD_RES 32: note = Amino group
  MOD_RES 8: note = alpha Methylcysteine
  MOD_RES 15: note = alpha Methylcysteine
  MOD_RES 18: note = alpha Methyllysine
  MOD_RES 20: note = 2-Aminoisobutyric acid
  MOD_RES 32: note = Hydroxyproline

SEQ ID NO: 30 - protein, 32 aa, synthetic construct; identical to SEQ ID NO: 15
  MOD_RES 1: note = Benzoyl
  MOD_RES 32: note = Amino group
  MOD_RES 18: note = alpha Methyllysine
  MOD_RES 20: note = 2-Aminoisobutyric acid
  MOD_RES 32: note = Hydroxyproline

SEQ ID NO: 31 - protein, 32 aa, synthetic construct; identical to SEQ ID NO: 27
  MOD_RES 1: note = Acetylation
  MOD_RES 32: note = Amino group
  MOD_RES 8: note = N Methylcysteine
  MOD_RES 15: note = N Methylcysteine
  MOD_RES 18: note = alpha Methyllysine
  MOD_RES 20: note = 2-Aminoisobutyric acid
  MOD_RES 32: note = Hydroxyproline

SEQ ID NO: 32 - protein, 32 aa, synthetic construct
  MOD_RES 1: note = Acetylation
  MOD_RES 32: note = Amino group
  MOD_RES 18: note = alpha Methyllysine
  MOD_RES 20: note = 2-Aminoisobutyric acid
  MOD_RES 32: note = Hydroxyproline
ASHLSTAKLGRLSAKLXXLXDYPQTDVGAGSX

SEQ ID NO: 33 - protein, 32 aa, synthetic construct
  MOD_RES 1: note = Acetylation
  MOD_RES 32: note = Amino group
  MOD_RES 32: note = Hydroxyproline
  MOD_RES 20: note = 2-Aminoisobutyric acid
ASHLSTAVLGKLSAELHKLXDYPQTDVGAGSX

SEQ ID NO: 34 - protein, 32 aa, synthetic construct; identical to SEQ ID NO: 27
  MOD_RES 1: note = Acetylation
  MOD_RES 32: note = Amino group
  MOD_RES 20: note = 2-Aminoisobutyric acid
  MOD_RES 18: note = alpha Methyllysine
  MOD_RES 8: note = Ornithine
  MOD_RES 15: note = Ornithine
  MOD_RES 32: note = Hydroxyproline

SEQ ID NO: 35 - protein, 32 aa, synthetic construct; identical to SEQ ID NO: 27
  MOD_RES 1: note = Acetylation
  MOD_RES 32: note = Amino group
  MOD_RES 32: note = Hydroxyproline
  MOD_RES 20: note = 2-Aminoisobutyric acid
  MOD_RES 18: note = alpha Methyllysine
  MOD_RES 15: note = 2,4 Diaminobutyric acid
  MOD_RES 8: note = 2,4 Diaminobutyric acid

SEQ ID NO: 36 - protein, 32 aa, synthetic construct; identical to SEQ ID NO: 33
  MOD_RES 1: note = Hydrogen
  MOD_RES 32: note = Amino group
  MOD_RES 32: note = Hydroxyproline
  MOD_RES 20: note = 2-Aminoisobutyric acid

SEQ ID NO: 37 - protein, 32 aa, synthetic construct; identical to SEQ ID NO: 33
  MOD_RES 1: note = Benzoyl
  MOD_RES 32: note = Amino group
  MOD_RES 32: note = Hydroxyproline
  MOD_RES 20: note = 2-Aminoisobutyric acid